(19) United States
(12) Patent Application Publication
(10) Pub. No.: US 2005/0037344 A1
(43) Pub. Date: Feb. 17, 2005

(54) Nucleic acid array

(75) Inventors: Bruno Stuhlmuller, Berlin (DE); Thomas Haupl, Erkner (DE)

Correspondence Address: WELSH & KATZ, LTD, 120 S RIVERSIDE PLAZA, 22ND FLOOR, CHICAGO, IL, 60606, US

(73) Assignee: PathoArray GmbH, Berlin (DE)

(21) Appl. No.: 10/278,698

(22) Filed: Oct. 23, 2002

(30) Foreign Application Priority Data
Nov. 9, 2001 (DE) 101 55 600.4-41

(51) Int. Cl. C12Q 1/68; C12M 1/34

(52) U.S. Cl. 435/6; 435/287.2

(57) ABSTRACT

It is proposed to use selectioned monocyte macrophage genes to provide tools for diagnostic, prognostic and therapy-monitoring analysis and for performing screenings for pharmacologically active substances and substance classes of chronic inflammatory diseases, chronic inflammatory diseases induced by bacteria, arteriosclerosis, tumors, organ and tissue transplantations, and sepsis when examining blood, tissue, purified or cultivated cells.

Tools for diagnostic, prognostic and therapy-monitoring analysis and for performing screenings for pharmacologically active substances and substance classes of chronic inflammatory diseases, chronic inflammatory diseases induced by bacteria, arteriosclerosis, tumors, organ and tissue transplantations, and sepsis when examining blood, tissue, purified or cultivated cells

This invention relates to tools for diagnostic, prognostic and therapy-monitoring analysis of chronic inflammatory diseases, chronic inflammatory diseases induced by bacteria, arteriosclerosis, tumors, organ and tissue transplantations, and sepsis when examining blood, tissue, purified or cultivated cells

Acute and chronic inflammatory processes in the blood and vascular system and in tissue may result in pathologic sediments, fibrotic transformation processes, and immediate destruction of tissues and organs.

The cells of the monocyte/macrophage system are greatly involved in the activation and maintenance of inflammatory cascades in blood and tissue as may occur in the course of inflammatory rheumatic diseases, bacteria-induced inflammatory diseases, tumors, organ and tissue transplantations, arteriosclerosis, and sepsis. Monocytes and macrophages are highly activated in the course of these diseases, display changes in the set of their surface molecules, contact other cells, and excrete certain messenger substances that keep the inflammatory process going. In addition to unspecific inflammatory reactions, specific stimulation of the immune system may occur.

Autoaggressive reactions can occur that are either mediated by the pathogen or have other causes (e.g. external, environmental factors such as radiation, toxins, or allergens) or may in part be caused by genetic predisposition. These may develop as excessive reactions in the course of the unspecific or specific inflammatory and defense reaction and result in damage to, or the destruction of, organ systems. Monocytes and macrophages are assigned a major part in these autoaggressive reactions, too.

The molecular processes in monocytes and macrophages that contribute to such a chronic inflammation and/or autoaggression are widely undetermined. There is an urgent need to study these (1) for diagnostic reasons, that is, classification and pathophysiological evaluation of the disease, (2) for prognostic reasons and optimum utilization of therapeutic options, (3) for therapeutic reasons to monitor an existing treatment or to develop new therapeutic approaches.

Current anti-inflammatory treatments for chronic inflammatory diseases from the rheumatic group of diseases are unspecific and often only have limited power to hinder the inflammatory process in its course and intensity. The diseases frequently progress until organs are damaged or even completely destroyed.

Autoimmune diseases and/or inflammatory diseases can in principle affect any organ system. The following list is exemplary: diseases of the skeletal and support structure (rheumatoid arthritis, reactive arthritis, Bechterew's disease, osteoarthritis and abrasion-related synovitis), of the colon (colitis ulcerosa and Crohn's disease), of the liver (autoimmunohepatitis, chronic viral hepatitis, primary biliary zirrhosis), endocrine organs (pancreas: juvenile type-I diabetes; thyroid gland: Hashimoto thyroiditis, Basedow's disease), of the skeletal muscles (polymyositis, or dermatomyositis when the skin is involved), the myocardium (rheumatic fever, dilatative cardiomyopathy, viral myocarditis), the skin (sclerodermia, psoriasis, neurodermitis), the lungs (pulmonary fibrosis, Goodpasture's syndrome, chronic obstructive lung disease, sarcoidosis), the brain or central nervous system (multiple sclerosis) and the cardiovascular system (vasculitis, arteriosclerosis) as well as chronic diseases of multiple organs (systemic lupus erythematosus, Sjögren's syndrome, systemic sclerosis, sepsis) and tumors.

Rheumatoid arthritis is a chronic inflammatory joint disease that results in progressive destruction of the affected joints. Both the monocytes circulating in the peripheral blood and the further differentiated cells of the monocyte/macrophage system that are present in the joint as synovial macrophages and dentritic cells show cell activation. This is reflected in comparable transcription patterns for proteins that are characteristic for inflammations (messenger substances, proteases, etc.). Rheumatoid arthritis could as yet be diagnosed using the clinical ACR criteria and by pathohistological examination of the affected tissue. The disease frequently was in a relatively advanced stage, and the joint already had suffered irreversible damage. Thus therapeutic measures are more often than not applied too late, when autoaggressive damage has been done and become irreversible.

It can be expected from studying gene expression profiles that a new molecular characterization of the disease will be possible and allow its classification into subgroups according to pathophysiological characteristics. In addition, the aggressive nature of the disease may be forecast. This would influence the selection and intensity of medication at an early point in time.

And regarding the as yet mainly unspecific therapeutic measures, it can be expected that knowledge of the molecular mechanisms of chronic inflammations will also point to specific candidates based on which treatment concepts can be developed. This can be achieved by specific biological substances, relevant antagonists, or by natural pharmacological substances or chemically acting specific substances that directly interfere with the inflammatory cycle of the monocyte/macrophage system.

Numerous areas with infiltrated cells of the monocyte/macrophage system containing characteristic gene expression patterns are found in tumors of the hemopoietic system or in tumor diseases with neoplastic changes. These gene expression patterns contain both inflammation-specific gene expressions and tumor-specific gene expressions.

With sepsis and bacteria-induced abrasion-related synovitis, cells of the monocyte/macrophage system are activated by bacterial infection, primarily via liposaccharides, and once again comprise a generally similar gene expression pattern as non-bacterial inflammatory diseases but also a specific gene pattern induced by bacteria. Thus the two types of abrasion-related synovitis without a bacterial trigger can be distinguished from bacteria-induced abrasion-related synovitis by their gene patterns.

In the case of arteriosclerosis, monocytes are already activated in the peripheral blood and excited to bind destroyed, inflammation-specific arterial regions. Communication by cell-to-cell contact with endothelial cells contributes to keeping activation and recruitment of the monocyte/macrophage system going.

Leading microarray manufacturers (Affymetrix, Clontech, Nanogene) currently offer commercial microarrays that contain from 4,000 up to 12,000 accidental genes in each set. Several sets that cover a total of 48,000 genes are required for an overall transcription analysis of all gene units. With a total number of ca. 40,000 genes (=ca. 120,000 individual gene variants) of the human genome, there is an extraordinary small coverage regarding the total number of different transcripts. Obviously this procedure is expensive and requires a major effort in terms of biometrical analysis while its hit quota is low when it comes to detecting disease-specific, cell-specific genes.

It is the problem of this invention to provide tools that are suited for diagnostic, prognostic, and therapy-monitoring analyses and for performing screenings for pharmacologically active substances and substance classes of rheumatoid arthritis, other chronic inflammatory diseases, infectious diseases, tumors, arteriosclerosis, organ and tissue transplantations, and sepsis.

This problem is solved by the characteristics listed in claim 1.

According to the invention, tools are to be used for this that use sequences of a selection of the genes listed below or sequences of all genes listed below, including other genes, or that use RNA that is complementary to the genes listed:

TABLE 1
Cytokines and factors and ligands:

| | |
|---|---|
| Interleukin-1α | (Acc. # NM_000575) |
| Interleukin-1β | (Acc. # NM_000576) |
| Interleukin-6 | (Acc. # AF372214) |
| Interleukin-8 | (Acc. # L19591) |
| Interleukin-10 | (Acc. # XM_001409) |
| Interleukin-13 | (Acc. # HSU62858) |
| Interleukin-15 | (Acc. # XM_003529) |
| Interleukin-16 | (Acc. # AF053412) |
| Interleukin-18 | (Acc. # E17135) |
| Angiopoietin-like factor (CTD6) | (Acc. # XM_001529, XM_042319) |
| Inhibin β-B (INHBB) | (Acc. # NM_002193) |
| Tumor necrosis factor-α | (Acc. # NM_000595) |
| Tumor necrosis factor -β | (Acc. # D12614) |
| Transforming growth factor-β (TGF-β) | (Acc. # XM_008912, NM_00660) |
| Latent TGF-β binding prot. LTBP4 | (Acc. # NM_003573, XM_008868) |
| Melanoma stimulating activity (MGSA) | (Acc. # X54489) |
| Chemokine Gro-α/MGSA | (Acc. # X12510, XM_003504) |
| Chemokine (C-X-C motif) ligand 16 | (Acc. # NM_022059) |
| Chemokine alpha-3 (CKA3) | (Acc. # NM_002993) |
| CC-Chemokine (SLC) | (Acc. # AB002409) |
| EBI-1-ligand chemokine | (Acc. # AB000887) |
| Small inducible cytokine subfamily A(SCYA21) | (Acc. # XM_048450) |
| Small inducible cytokine (SCYA21) | (Acc. # NM_002989) |
| Megakaryocyte stimulating factor | (Acc. # U70136) |
| Monocyte colony stimulating factor (M-CSF) | (Acc. # NM_000757) |
| Granulo-/Monocyte colony stimu. factor (GM-CSF) | (Acc. #: E01817) |
| Macrophage inflammatory Protein (MIP-1) | (Acc. # HUMMIP1A) |
| Makrophage inflammatory Protein (MIP-2 | (Acc. # AF106911) |
| Monocyte migration inhibitory factor (MIF) | (Acc. # L19686) |
| Monocyte tissue factor | (Acc. # M16553) |
| Monocyte chemoattractant protein-1 (MCP-1) | (Acc. # S71513) |
| Monocyte chemoattractant protein-2 (MCP-2) | (Acc. # NM_005623) |
| Monocyte chemoattractant protein-3 (MCP-3) | (Acc. # X72308; S57464) |
| Fraktalin small inducible cytokine | (Acc. # NM_002996) |
| Stromal derived factor-1 (SDF-1) | (Acc. # HSU16752) |
| Insulin-like growth factor-5 bind. Protein | (Acc. # NM_000599) |

Receptors, ion channels and associated proteins:

| | |
|---|---|
| Angiotensin receptor-II homolog (ATR-IIh) | (Acc. # L48211) |
| Toll-like receptor-2 | (Acc. # XM_003304) |
| Toll-like receptor-4 | (Acc. # XM_005336) |
| Opoid receptor kappa | (Acc. # XM_011716) |
| Interleukin-1 receptor | (Acc. # XM_002686) |
| Interleukin-2 receptor α-subunit | (Acc. # XM_043149) |
| Interleukin-2 receptor β-subunit | (Acc. # XM_009962, M26062) |
| Interleukin-2 receptor γ-subunit | (Acc. # XM_047675) |
| Interleukin-7 receptor | (Acc. # AH007043, NM_008372) |
| Interleukin-8 receptor α (IL8RA) | (Acc. # XM_058007) |
| Interleukin 8 receptor β (IL8RB) | (Acc. # NM_001557 |
| Fc-receptor-I | (Acc. # J03619, AF200220) |
| Fc-receptor-II | (Acc. # M28696, M28697) |
| Fc-receptor-III | (Acc. # Z46223, Z46223) |
| Tumor necrosis factor-α receptor | (Acc. # S63368) |
| C-Chemokine (C-C motif) receptor-5 (CCR5) | (Acc. # NM_000579, XM_030397) |
| C-Chemokine (C-C motif) receptor-7 (CCR7) | (Acc. # XM_049959) |
| Chemokine-X-C-receptor-4 (CXCR-4) | (Acc. # NM_003467) |
| Progesterone recept.-assoc. immunophilin(FKBP54) | (Acc. # U42031) |
| Partial p58 gene for NK receptor | (Acc. # AJ000542) |
| Vascular endothelial growth factor | (Acc. # AY047581) |
| Vascular endothelial growth factor-β | (Acc. # BC008818) |
| Calcium activated potassium channel (KCNN3) | (Acc. # AF031815, AY049734) |
| G protein-coupled cytokine receptor EBI1 | (Acc. # L31581) |
| G protein-coupled cytokine receptor EBI3 | (Acc. # XM_012857, L08187) |
| EBI3-associated protein | (Acc. # U41806) |

Membrane proteins and associated proteins:

| | |
|---|---|
| CD14 | (Acc. # XM_003822) |
| CD68 | (Acc. # XM_008237) |
| CD69 | (Acc. # BC007037) |
| CD11b | (Acc. # J03925) |
| Adhesion receptor CD44 | (Acc. # M31165) |
| Actin binding coronin-like protein (HCORO1) | (Acc. # U34690) |
| Integral membrane protein | (Acc. # L32185) |
| Epithelial membrane prot.-3 (EMP-3)/HMPMP-1 | (Acc. # X94771, U87947) |
| Mac-2 binding protein | (Acc. # L13210) |
| Integral membrane protein E16 | (Acc. # M80244) |
| HLA-D II beta chain | (Acc. # X03066) |
| Desmin | (Acc. # HSU59167, XM_002601) |
| Fibronectin precursor | (Acc. # X02761) |
| Adducin 1α | (Acc. # X58141, NM_014190) |
| HLA DRB1 | (Acc. # X88971) |
| Integrin-α 5 subunit | (Acc. # X06256) |
| Integrin cytopl. domain assoc. protein (Icap-1α) | (Acc. # AF012023) |
| Integrin cytopl. domain assoc. protein (Icap-1β) | (Acc. # AF012024) |
| Titin | (Acc. # X69490, NM_003319) |
| Thrombospondin-1 (TSP-1) | (Acc. # XM_007606) |
| Semaphorin-3 | (Acc. # AB000220) |
| Semaphorin-F homolog | (Acc. # U52840) |
| TSP-2 | (Acc. # NM_003247) |
| TSP-1/Semaphorin-5a Homolog | (Acc. # NM_003966) |
| VCAM-1 | (Acc. # X53051) |
| Periplakin (PPL) | (Acc. # XM_032727, NM_002705) |
| Envoplakin (EVPL) | (Acc. # XM_008135) |
| Peripheral myelin protein 22 (PMP-22) | (Acc. # XM_052499) |

(Proto)-onco-, tumor-suppressor-, differentiating genes & assoc. proteins:

| | |
|---|---|
| H19 RNA | (Acc. # M32053) |
| Tumor suppressor Brush-1 | (Acc. # S69790) |
| Pim-2 protooncogene | (Acc. # U77735, XM_010208) |
| HOX-B3 | (Acc. # N70814) |
| MEL-18 | (Acc. # D13969) |
| c-fos | (Acc. # V01512) |
| c-jun | (Acc. # NM_002229) |
| c-myc | (Acc. # AH001511) |
| c-myc related oncogene (pHL-1) | (Acc. # X54629) |
| c-Ret tyrosine kinase receptor ligand 2 (RETL2) | (Acc. # U97145) |
| c-Ret tyrosine kinase receptor ligand 1 (RETL1) | (Acc. # U97144) |
| jun-B | (Acc. # XM_009064) |
| c-Jun activation domain binding protein | (Acc. # U65928) |
| Desmoyokin/AHNAK | (Acc. # X74818, M80899) |
| Rad mRNA | (Acc. # L24564) |
| PTEN | (Acc. # AH005966, XM_005867) |
| c-ras homolog gene family, member B (ARHB) | (Acc. # XM_002689, NM_004040) |
| Transforming activity oncogene (TRE-2) | (Acc. # X63596) |
| Transforming activity oncogene (TRE-17 | (Acc. # HSTRE213) |
| Kruppel-like fetal globin gene activator (FKLF) | (Acc. # AF272830) |
| c-fos related antigen (fra-1) | (Acc. # X16707) |
| c-fos related antigen (fra-2) | (Acc. # X16706) |

Acute phase protein:

| | |
|---|---|
| Large ferritin subunit | (Acc. # M11146) |
| Small ferritin subunit | (Acc. # NM_000146) |

Enzymes, enzyme-associated proteins and inhibitors:

| | |
|---|---|
| Activation-induced cytidine deaminase | (Acc. # AB040431, NM_020661) |
| Phospholipase-C | (Acc. # XM_041310) |
| Prostaglandin G/H Synthase | (Acc. # S36271) |
| Prostaglandin-Endoperoxide Synthase-1 | (Acc. # NM_000962) |
| Cyclooxygenase-1 | (Acc. # HSU63846) |
| Cyclooxygenase-2 | (Acc. # M90100) |
| Endothelin-1 (EDN1) | (Acc. # NM_001955) |
| Endothelin-1 (EDN2) | (Acc. # NM_001956) |
| Clustrin (complement lysis inhibitor, SP-40, 40) | (Acc. # XM_027447, X14723) |
| Fatty acid desaturase 1 (FADS1) | (Acc. # AF084558) |
| Cysteine dioxygenase 1 (CDO-1) | (Acc. # U80055) |
| Histidine biosynthesis protein | (Acc. # NM_007016) |
| Chitinase 1 | (Acc. # NM_003465) |
| Chitinase precursor | (Acc. # AF290004) |
| L-glycerol-3-phosphat: NAD oxidoreductase | (Acc. # L34041) |
| Alcohol dehydrogenase class I gamma subunit | (Acc. # M12272) |
| Procarboxypeptidase B1 | (Acc. # NM_001871) |
| Phosphoenolpyruvate carboxykinase (PCK1) | (Acc. # XM_009672, L05144) |
| Lysozyme | (Acc. # BC004147) |
| Transaldolase | (Acc. # NM_006755) |
| Thymosin-β4 | (Acc. # M17733) |
| Metallothionein 1L (MT1L) | (Acc. # NM_002450) |
| Manganese-superoxide dismutase (Mn-SOD) | (Acc. # S77127) |
| Superoxide dismutase 1 | (Acc. # K00065) |
| Superoxide dismutase 2 | (Acc. # NM_000636) |
| Superoxide dismutase 3 | (Acc. # NM_003102) |
| Copper/zinc-superoxide dismutase (Cu/Zn-SOD) | (Acc. # M13267) |
| Catalane | (Acc. #) |
| Monoamine oxidase-A (MAOA) | (Acc. # M68840, XM_055485) |
| Fatty acid synthetase | (Acc. # U29344) |
| Glutathion peroxidase | (Acc. # X13710) |
| Glutathion peroxidase 3 | (Acc. # NM_002084) |
| Glucocerebrosidase | (Acc. # M16328) |
| Inducuble nitric oxide synthase | (Acc. # AB022318) |
| Transglutaminase 1 (K polypeptide) | (Acc. # XM_007310) |
| Transglutaminase (TGase) | (Acc. # M55153, SEG_HUMETG)* |
| α-1-Antitrypsin | (Acc. # HSATPR1) |
| Protein tyrosin phosphatase | (Acc. # U27193) |
| Carbonic anhydrase precursor (CA 12) | (Acc. # AF037335) |
| Metallothionein-IG gene (MT1G) | (Acc. # J03910) |
| Lymphocyte phosphatase assoc. protein (LPAP) | (Acc. # X97267, AA011257) |
| Flap endonuclease 1 DNA repair gene (FEN1) | (Acc. # AC004770) |
| Flap structure-specific endonuclease 1 (FEN1) | (Acc. # L37374, XM_043386) |

Kinases, protein kinases (PKN) and PKN inhibitors:

| | |
|---|---|
| Protein kinase C-alpha subunit | (Acc. # X52479) |
| Protein kinase C-beta-1 subunit | (Acc. # XM_047187) |
| Protein kinase C-beta-2 subunit | (Acc. # M13975) |
| Protein kinase C-gamma subunit | (Acc. # M34182) |
| Protein kinase C-delta subunit | (Acc. # D10495) |
| Protein kinase-C inhibitor | (Acc. # U51004 |
| Ik-Kinase-κ | (Acc. # AF029684 |
| PI3-Kinase | (Acc. # Y13892) |
| MAP Kinase-11 | (Acc. # XM_035889) |
| p38 MAP kinase | (Acc. # AF031135) |
| p38 MAP kinase interacting protein | (Acc. # XM_035930) |
| Serin/threonin kinase | (Acc. # AB015982) |
| Tyrosine kinase-1 | (Acc. # XM_002037) |
| Tyrosine kinase-2 | (Acc. # XM_005480) |
| Non-receptor protein tyrosine kinase tyk2 | (Acc. # X54637) |
| Mitogen- and stress-activated protein kinase-1 | (Acc. # AF074393) |
| Mitogen- and stress-activated Protein Kinase-2 | (Acc. # AF074715) |
| Casein kinase 1, alpha 1 (CSNK1A1) | (Acc. # NM_001892, L37042) |
| Tyrosine kinase 1 (TIE-1) | (Acc. # XM_002037) |
| Tyrosine kinase 2 (TIE-2) | (Acc. # XM_005480) |

Differentiation genes:

| | |
|---|---|
| WNT-6 | (Acc. # AY009401, AB059570) |
| WNT-13 | (Acc. # Z71621) |
| BMP-4 | (Acc. # M22490) |

Proteinases, matrix metalloproteinases (MMP) and MMP-inhibitors:

| | |
|---|---|
| Cathepsin-B | (Acc. # XM_035662) |
| Cathepsin-G | (Acc. # M16117) |
| Cathepsin-K | (Acc. # NM000396) |
| Cathepsin-L | (Acc. # NM_001912) |
| Cathepsin-S | (Acc. # M86553) |
| Matrix metalloproteinase-1 (MMP-1) | (Acc. # NM_002421) |
| MMP-3 | (Acc. # X05232) |
| MMP-9 | (Acc. # XM_009491) |
| Disintrigin protease | (Acc. # Y13323) |
| Tissue inhibitor of MMP type 1 (TIMP-1) | (Acc. # NM_003254) |
| TIMP-2 | (Acc. # NM_003255) |
| TIMP-3 | (Acc. # NM_000362) |
| TIMP-4 | (Acc. # NM_003256) |
| Serin protease-like mRNA | (Acc. # M17016) |

Apoptosis and cell cycle regulators:

| | |
|---|---|
| Annexin A-2II | (Acc. # BC001388) |
| Growth arrest DNA-damage-induc. prot. (GADD45) | (Acc. # M60974) |
| Growth arrest DNA-damage-induc. prot.α(GADD45A) | (Acc. # XM_056975, XM_040594) |
| Growth arrest DNA-damage-induc. prot.β(GADD45B) | (Acc. # NM_015675, AF087853) |
| Growth arrest DNA-damage-induc. prot.g(GADD45G) | (Acc. # NM_006705) |
| Lymphocyte G0/G1 switch gene (GOS-3) | (Acc. # L49169) |

Signal transduction regulators:

| | |
|---|---|
| STAT-1 | (Acc. # NM_007315) |
| STAT-4 | (Acc. # XM_002711) |
| Adenylate kinase 1 (AK1) | (Acc. # NM_000476) |
| Inositol 1,4,5-trisphosphate 3-kinase (ITPKC) | (Acc. # XM_047369, XM_047368) |
| Phosphatidylinositol-3′-kinase (PI3K) | (Acc. # Y11312) |

Transcription factors, translation factors and associated proteins:

| | |
|---|---|
| Transcription factor AREB6 | (Acc. # D15050) |
| Transcription factor 8 (TCF8) | (Acc. # XM_030006) |
| Nuklear factor kappa-B | (Acc. # M58603) |
| AP-1 | (Acc. # AB015319, AB015320) |
| PU.1 | (Acc. # X66079) |
| SPI-B | (Acc. # X66079) |
| v-maf musculoaponeurotic fibrosarcoma (MAFF) | (Acc. # XM_039249, XM_039250) |
| Zinc finger transcription factor (GKLF) | (Acc. # AF105036, AK026253) |
| Zinc finger protein | (Acc. # M80583) |
| CCAATA enhancer binding protein-beta | (Acc. # NM_005194) |
| RNA-polymerase II elongation factor | (Acc. # L47345) |
| Translation elongation factor-1 α-1 (EEF1A1) | (Acc. # BC009733) |
| Translation elongation factor-1 α-2 (EEF1A2) | (Acc. # XM_028863) |
| Translation elongation factor 2 (EEF2) | (Acc. # NM_001961) |
| L1-Element (L1.20) | (Acc. # U93569) |
| Leukemia zinc finger PLZF | (Acc. # AF060568) |
| Activating transcription factor 3 (ATF3) | (Acc. # XM_016795, XM_034219) |
| Zinc finger transcriptional regulator (GOS-24) | (Acc. # M92843) |
| TGF-β-inducing early growth response 2 | (Acc. # AA427597) |
| SP1-like zinc finger transcript. factor (TIEG2) | (Acc. # AF028008) |
| snRNA activating protein complex | (Acc. # AF032387) |
| oct-binding factor-1 (OBF-1) | (Acc. # Z49194) |
| Early growth response protein 1 (EGR-1) | (Acc. # R75775) |

Ribosomal/ribonuclear regulator genes and associated proteins:

| | |
|---|---|
| hnRNP pseudogen(gp43) (position: 97.026-98.073) | (Acc. # AL034397) |
| Ribosomal protein L19 | (Acc. # XM_002758) |
| Ribosomal protein S13 | (Acc. # XM_039215) |
| Histon-H1 (0) family mRNA | (Acc. # X03473) |
| H4-histone family, member H (H4FH), mRNA | (Acc. # NM_003543) |

Others:

| | |
|---|---|
| IER-3 | (Acc. # NM_003897) |
| Endoplasmatic glycoprotein Gp36 | (Acc. # U10362) |
| Natural resist.-assoc. macroph.protein (Nramp1) | (Acc. # D50402) |
| Calgranulin - S100A12 protein | (Acc. # XM_001682, NM_005621) |
| 14-3-3 gamma protein | (Acc. # AF142498) |
| Serum amyloid-A | (Acc. # M81349, M81451) |
| GDF-1 | (Acc. # NM_001492) |
| Solute carrier family 7 mRNA (SLC7A5) | (Acc. # NM_003486) |
| PLAB/MIC-1 | (Acc. # NM_004864) |
| EAP-(HBp15/L22) | (Acc. # NM_006755) |
| Small proline-rich protein-1 | (Acc. # L05187) |
| NAG-1 | (Acc. # AF173860) |
| BST-1 | (Acc. # D21878) |
| II56KD | (Acc. # M24594) |
| Fibulin-1 D | (Acc. # NM_006486) |
| Nebulin | (Acc. # XM_040435) |
| VDUP1 upregulated by 1,25-dihydroxyvitamin D-3 | (Acc. # XM_002093, XP_002093) |
| Tumor necrosis factor stimulated gene (TSG-6) | (Acc. # NM_007115) |
| Tumor necrosis factor stimulated gene (TSG-37) | (Acc. # M31164) |
| Osteopontin | (Acc. # AF052124) |
| Tristetraproline (TTP) | (Acc. # M63625) |
| Nephropontin | (Acc. # M83248) |
| Tonsillar lymphocyte LD78 mRNA | (Acc. # X03754) |
| MB-1 gene (CD79a-B cell) | (Acc. # U05259) |
| Human glycoprotein (gp39) | (Acc. # M80927, Y08374) |
| Glia derived nexin precursor | (Acc. # AI743134) |
| Heat shock protein 70B (HSP-70B) | (Acc. # X51757) |
| Apolipoprotein D | (Acc. # XM_049984, XM_003067) |
| Dead box, Y isoform (DBY), altern.transcr. 2 | (Acc. # AF000984) |
| Myocilin (GLC1A) | (Acc. # AH006047) |
| DR1-associated corepressor (DRAP1) | (Acc. # U41843) |
| DR1-associated protein 1 (neg. cofactor 2 α) | (Acc. # XM_055156) |
| FK506 bind.-12-rapamycin assoc.prot.1 (FRAP1) | (Acc. # XM_001528, XM_042283) |
| Microfibril-associated glycoprotein-2 (MAGP-2) | (Acc. # AH007047, NM_003480) |
| Adrenomedullin (ADM) precursor | (Acc. # NM_001124, XM_051743) |
| DNA-damage-inducible transcript 3, clone MGC: 4154 | (Acc. # BC003637) |
| Calretinin - calcium binding protein | (Acc. # X56667) |
| Breakpoint cluster region (BCR) mRNA | (Acc. # XM_017097) |
| Adipose most abundant gene transcript 1 (APM1) | (Acc. # NM_004797, XM_003191) |
| Novel adipose specific collagen-like factor | (Acc. # D45371) |

Genes and ESTs with unknown functions:

| | |
|---|---|
| IMAGE 745750 | (Acc. # AA420624) |
| KIAA0935 | (Acc. # AB023152) |
| KIAA0618 | (Acc. # AB014518) |
| Homolog to FLJ23382 fis clone HEP16349 | (Acc. # AK027035) |
| Hypothetical gene mRNA | (Acc. # XM_005331) |
| HDCMB07P/PCM-1 | (Acc. # AF068293) |
| cDNA clone DKFZp762M2311 | (Acc. # AL512760) |
| cDNA clone PP2684 | (Acc. # AF218004) |
| cDNA clone MGC: 1811 (IMAGE: 3506276) | (Acc. # BC015961) |
| cDNA clone IMAGE: 979127 | (Acc. # AA522530) |
| cDNA clone IMAGE: 4279495 5′, mRNA sequence | (Acc. # Bf667722) |
| cDNA clone 137308 mRNA, partial cds | (Acc. # U60873) |
| cDNA clone IMAGE: 159541 | (Acc. # H15814) |
| cDNA clone MAMMA1001272 | (Acc. # AU147646) |
| cDNA clone IMAGE: 2419382 | (Acc. # AI826771) |
| cDNA clone IMAGE: 3941411 | (Acc. # BE797145 |
| cDNA clone IMAGE: 3834583 | (Acc. # BE743390) |
| cDNA clone IMAGE: 4565371 | (Acc. # BG397372) |
| cDNA clone MGC: 2460 IMAGE: 2964524 | (Acc. # BC009504) |
| cDNA clone RC3-HT0585-010400-013-all HT0585 | (Acc. # BE176664) |
| cDNA clone similar to CG8974 gene product | (Acc. # XM_018516) |
| cDNA clone BSK-65 | (Acc. # W99251) |
| cDNA clone IMAGE: 3844696 | (Acc. # BE730665) |
| FLJ23382 fis, clone HEP16349 | (Acc. # AK027035) |
| FLJ20500 fis, clone KAT09159 | (Acc. # AK000507, BC015236) |
| GABBR1 region of AL031983 | (Acc. # 12329558) |
| cDNA clone CS0DE006YI10 5′ prime end | (Acc. # AL541302) |
| cDNA clone CS0DE006YI10 3′ prime end | (Acc. # AL541301) |
| EST371586 IMAGE resequences | (Acc. # Aw959516) |
| MEN1 region clone epsilon/beta | (Acc. # Af001892) |

Control genes for quantification adjustment:

| | |
|---|---|
| alpha-Aktin | (Acc. # M20543) |
| beta-Aktin | (Acc. # XM_037239) |
| gamma-Aktin | (Acc. # NM_001614) |
| Glyceraldehyde-3-phosphate-dehydrogenase | (Acc. # XM_033258) |
| Glucose-6-phosphate-dehydrogenase | (Acc. # XM_013149) |
| 28S rRNA | (Acc. # M27830) |
| 18S rRNA | (Acc. # M10098) |

TABLE 2

BSK-66 or accession # AA393029
CGGTTGGGGCTCTGGTCTTGGATTTGATGTGTGGCGAAGGCTGCAATTGTTTAATAA
CCCTTCATGATTCAACAGCTCTTCAAGAACTTTCCTCTGTTCTTGTGTGGAGCTCGT
GACAGCCAGTGGTGGTGGAGCTCCAGCCCTCTCTTCCCACAGGCACAAGCCGGGTTC
CTGAGTCCCAGGGCTTCTCGGGAGGTGTCTGCCCTCCTCTTTCAGACACCCTCTGCC
CTGTGTCCCAGGGCCCTGGGCCTGTGCTGCACTGAGCAGAGACTGTAGGGGACCGGC
TCTCCCACTCCTCCCAGATGGGCAGCGTCTTCCGTGTCGGGAGCATGCTGTGCTGCT
TTTCTCTTCTCAGTCTCTTAGTTTTTGCGGGTTCTTACGCATGTGAGGTGTGGACTT
GCATGGTGGGGAGCTCAAATGGTACATGAAGGGGAGGAGCCCTCTGAGTGCTGTGAT
TTGTTCCATCATTACCGCTTCCTGATCACGGTGACCTGCACTGCTGGAGTGGTCAGT
GGAGCCAGGCCTCCCCACAACAGTGTTCCCATCGCCTTCTTACTATTGATTTCTATT
CTTAAAATATTGTATTACTTAGCACTCTTTTGAAGACGTTCCAGTATATATCAAATG
ATCAAAAGTCCATAACCTTGTCCTACGTAGAAGCCAAAGGTGTCATGCAGTTTCAGG
TGTTCGAGTTTCCAGAATTCTTGTGATGACATTTGTAGGATTCTTCTTTTAGACTTG
GACCAAATTCTGTAACCTAATATTTGTCCTTCAGATTGACAGAGAACCGCAGGCAGG
TGTTTTCTCTGTCACACGTGTGGTGGGTGGCATCCTGGTGACATAAAGAATTGCCTT
TGGTAACTTGCCCAGAAGGCTGTAGGCTTATTTTCTGCTTAGACTTTCCCCTATTTC
TTTCTTTTCTTTTCTCG
BSK-89 or accession # AA574456 - forward
ATTTTAGGGACGTAGTAGATGATTTTTAGCGAATTTGATCGGCCAGAAGAACATACA
ATGGATTGGGACAAAGTCTGTTGGGCAGACAATGGTTTGTGACAAAATTCTGTCCAG
GTGTGTTGACCGAATTCAGGCTTTCTTTATGCGATATGAGTTCAGTTAATGAAAACA
CAGGGGAGTGACCAGAAGTGATTGTTTCCTTCTTTGGCGTTTCTGTCTTCCTCCTTT
TTTGTTCTATTCCCTTATTTTGCAACCTTTTGGATGTTACCCTTTGGAAGTTACCCT
CTTGTAACTTCCACATTAAAAGTTTGGGGGCTGGCTGATANAAGGAACTCCAGAGAA
CAACTTGATTCTCTGCTTTGGGAGAGACAGANAAATGAGGGGTGTCGAGGAAGGTCA
GANACACCCTGAGGCCTCTGCCTNCTTCAGCATGTCANAGCACCCTATTTTGGGGCT
TGCTTTCTGAGCCCNAACATCTCCACCCTTCCANGANTCTGTGGCTTATCCTTCCCA
ANGATAGGATCACTTGNCACTCTACTGANCCTAAGTTGTATTCANTTTCTTTTGATC
CGCCTNGACTCTNTAGCNANTGANAANCACAACNTGGNAACNAACCCTCATAAANCT
GCTNTANCTTCTGGTTTTAAGNNCAAAACA
BSK-89 or accession # AA574456 - reverse
CGCACATGTGGTGGCTACTAGTTCCTGGAATAGCAAAGCAATTCCTACCTCCATGCC
TTTTCAGTTGCTTTAGATAAGACCTGTTTTGCACCTGAGAAAACAGAAAGATCAGAG
CAGCTTTTTTGTGTGTTTTGTTTACACAGTGTTGTTGTATTTTCATTTGCTCTAGAC
TTCAAGGCAGATGCAAAAGAAAATGAAAAACAACTTAGGTTCAGTAGACTGGTCAAG
TGATCCTATTCCTTTGGCCAAGGATATGCCACAGACTCCTGGAAGGCTGGAGATGTT
GGGGCTCAGAAAGCAATACCCCAAAATAGGGTGCTTTGACATGCTCAAGTGAGGCAG
AAGCCTCACGGTCTCTCTCACCTTCCTCCACACCCCTCATTTCTCTGTCTCTCCCAA
AGCACAGAATCAAGTTGTTCTCTGGAGTTCCTTCTATCTGCCAGCCCCCAAACTTTT
AATGTGGAAGTTACAAGACGGTAACTTCCAAAGGCTAACATCCAAAAGGTTGCAAAA
TAAGGGAATAGAACAAGAAAGGAGGAACACAGAGACGCCAAAGAACGAAACAATCAC
TTCTGGTCACTCCCCTGTGTTTTCATTAACTGAACTCATATCGCATAAAGAAAGCCT
GAATTCGGTCGACACACCTGGACAGAATTTTGTCACTAACCATTGTCTGCCCAACAG
ACTTTGTCCCAATCCATTGTATCTTCTTCAGGCCCATCAAATTCCCTAAAAATCATC
TACTACCTCCCTAAAAT
BSK-67 or accession # AA574454
CGGTCAACCCAACACAGGCATGCTCATAAGGAAAGGTTAAAAAAAGCAAAAGGAACT
CGGCAAATCTTACCCCGCCTGTTTACCAAAATCATCACCTCTAGCATCACCAGCATT
AGAGGCACCGCCTGCCCAGTGACACATGTTTAACGGCCGCGGTACCCTAACCGTGCA
AAGGTAGCATAATCACTTGTTCCTTAAGTAGGGATCGGCTTGAATGGCTCCACGAGG
GTTCAGCTGTCTCTTACTTTTAACCAGTGAAATTGACCTGCCCGTGAAGAGGCGGGC
ATAACACAGCAAGACGAGAAGACCCTATGGAGCTTTAATTTATTAATGCAAACAGTA
CCTAACAAACCCACAGGTCCTAAACTACCAAACCTGCATTAAAAATTTCGGTTGGGG
CGACCTCGGGGCAGAACCCAACCTCCGAGCAGTACATGCTAAGACTTCACCAGTCAA
AGCGAACTACTATACTCAATAGATCCAATAACTTGACCAACGGAACAAGTTACCCTA
GGGATAACAGCGCAATCCTATTCTAGAGTCCATATCAACAATAGGGTTTACGACCTC
GATGTTGGATCAGGACATCCCAATGGTGCAGCCGCTACTAAAGGTTCGTTTGTTCAA
CGATTAAAGTCCTACGTGATCTGAGTTCAGACCGGAGTAATCCAGGTCGGTTTCTAT
CTACTTCAAATTCCCG
BSK-80 or accession # AA574455
CGTTCACTCCTGCCTGGGCAACGAAGAGTGAAACTCCATCTCAAAACAAACAAACAA
ACAAACAAACAAGCAAAAGCACTCTAGGTATAAAAGGAACTTCCTCTGCCTGCAAGA
CCCCCCATCTCCTTCCCATGAAGTCCTTAACTTCTGTTCAACCCAAACGCCCAGCGC
GTCCCTTCCACTGCGCTGCCCGATGCACCTCTGCCCGCCACGCCTTCAGTGTTGTGG
TCATTTGTGCCTGCGCACCCAGGGCTGCAGGTACCTTCCTCCAGTGTGCTTCCAGGA
CGGGTTATTCAGGATGCTGAGACGAGCCGCCAGCTTCACACAGAACTGGGGTGAGAC
CTCAGCACCTGCTGCCTGTGTTCCTGAGGCTGTCTGCCAAGGCGCTCAGGAAACGCA
CATGCCTCCTGACCCTCATATGCACACCTCGTGGACGGCAGCCTGCAGGACCACTGG
CAAGTTTTGTTGCCGAAATCCCTCTTCGAGGAAAAAAGTCAATTGTTGGCAATTAGA
TATTAAGATCACATAACTCACTTCAATCAGTCGTCTAAAAACAAACGGCTGATACCA
CTGAGTCTCAAGGAAGCAGCCACAGGGGCTTCTGCTGAGGGGGCAGGCGGAGCTTGA
GGAAACCGCAGATAAGTTTTTTTCTCTTTGAAAGATAGAGATTAATACAACTACTTA
AAAAATATAGTCAATAGGTTACTAAGATATTGCTTAGCGTTAAGTTTTAACGTAATT
TTAATAGCTTAAAATTTTAAGAGAAAATATGAAGACTTAGAAGAGTACCATGAGGAA
GGAAAAGATAAAAGGTTTCTAAAACATGACGGAGGTTGAGATGAAGCTTCTTCATGG
AGTAAAAAATGTATTTAAAAGAAAATTGAGAGAAAGCG
BSK-83 or accession # AI046025
GTTTTTAGGGTTCTTTAGTGTTGTTTCTTTCACCCAGGGGTGGTGGTCCCAGCCAGT
TTGGTGCTGACGGTGAGAGGAAATTAGAATCTGTTTGCAAATTGTCCAACCCACCCC
CTCAACATGAGGGGCTTCCATTTTCTGTGTTTTGTAAGGGAACTGTTTCCTTCATGC
CGCCATGTTCCTGATATTAGTTCTGATTTCTTTTTAACAAATGTTATCATGATTAAG
AAAATTTCCAGCACTTTAATGGCCAATTAACTGAGAATGTAAGAAAATTGATGCTGT
ACAAGGCAAATAAAGCTGTTTATTAACCTTG
BSK-83-2 - forward
GTTCAAACAGCAAACGCCCACAGATGGCCCAGAGGTGGTGGTAGTCAGGGTGTGTGG
GTGTTTTTAGGGTTCTTTAGTGTTGTTTCTTTCACCCAGGGGTGGTGGTCCCAGCCA
GTTTGGTGCTGACGGTGAGAGGAAATTAGAATCTGTTTGCAAATTGTCCAACCCACC
CCCTCAACATGAGGGGCTTCCATTTTCTGTGTTTTGTAAGGGAACTGTTTCCTTCAT
GCCGCCATGTTCCTGATATTAGTTCTGATTTCTTTTTAACAAATGTTATCATGATTA
AGAAAATTTCCAGCACTTTAATGGCCAATTAACTGAGAATGTAAGAAAATTGATGCT
GTACAAGGCAAATAAAGCTGTTTATTAACCTTG
BSK-78 -3- forward
ATCAACTTTCGATGGTAGTCGCCGTGCCTACCATGGTGACCACGGGTGACGGGGAAT
CAGGGTTCGATTCCGGAGAGGGAGCCTGAGAAACGGCTACCACATCCAAGGAAGGCA
GCAGGCGCGCAAATTACCCACTCCCGACCCGGGGAGGTAGTGACGAAAAATAACAAT
ACAGGACTCTTTCGAGGCCCTGTAATTGGAATGAGTCCACTTTAAATCCTTTAACGA
GGATCCATTGGAGGGCAAGTCTGGTGCCAGCAGCCGCGGTAATTCCAGCTCCAATAG
CGTATATTAAAGTTGCTGCAGTTAAAAAGCTCGTAGTTGGATCTTGGGAGCGGGCGG
GCCGGTCCGCCGCGAGGCGAGCCACCGCCGTNCNCGCCCTTGCCTCTCGGCGCCCCC
TNGATGCTCTTAGCTGANTGTCCCGCGGGGCCCGANCCGTTTACTTTGAAAAAATTT
NAGTGTTAAAGCANGCCCGAACCGCTGGATACCCGNNNTAGGAATAATCGATTANGA
CCNNGGNNCTNTTTGNNGGTTTCNGACTGAGCCNTATTAANANGGAC
J-4 or accession # AI046024
AAAACGACGGCCAGTGAATTGTAATACGACTCACTATAGGGCGAATTGGGCCCTCTA
CATGCATGCTCGAGCGGCCGCCAGTGTGATGGATATCTGCAGAATTCGGCTTTTGAC
ACCAGACCAACTGGTAATGGTAGCGACTGGCGCTCAGCTGGAATTCCGCCTGGGACT
ACCGGGTCTCACTCCAGAAGAGGCTTCTTCAGAGCATGGTAGTCTTGGGGTTCTAAC
AGAATGAGAGTAGAAGCTGCAAAACCTCTTGAAACTGGGGCTTGGGAGTCACACATG
ACTTTCTCCACATTCTGTTCGTCAAAAGCGAATCATAAGGACAGCACAGACTCAAGG
GATAAG
M-3 or accession # AI048523
AAACGACGGCCAGTGAATTGTAATACGACTCACTATAGGGCGAATTGGGCCCTCTAG
ATGCATGCTCGAGCGGCCGCCAGTGTGATGGATATCTGCAGAATTCGGCTTTTKACA
CCAGACCAACTGGTAATGGTAGCCACCGGTTCTCAGCTGGAATTCCGGATTGGTCCA
ATTGGGTATGAGGAGTTCAGTTATATGTTTGGGATTTTTTAGGTAGTGGGTGTTGAG
CTTGAACGCTTTCTTAATTGGTGGCTGCTTTTAGGCCTACTATGGGTGTTAAATTTT
TTACTCTCTCTACAAGGTTTTTTCCTAGTGTCCAAAGAGCTGTTCCTCTCTTGGACT
AACAGTTAAATTTACAAGGGGATTTAGAGGGTTCTGTGGGGCAAATTTAAAGTTGAA
CTAAGATTCTATCTTGGACAACCAGCTATCACCAGGCTCGGTAGGTTTGTTGCCTCT
NCCTATAAATCTTCCCACTATTTTTGTACATAGACGGGTGTTCTCTTTT
HOX-B3
GTTCAGTAATTATCTTTTATTTCATTTTCTCCCCTTCCCACCCCTCCCCCTCGGATC
CAGCAGAGGGCTGTGGTGGCGGCGGCGTCCAAGCGGGCGCGGACGGCGTACACGAGC
GCGCANTGGTGGAGCTGGAGAAGGAGTTCCATTTTAACCGCTACCTGTGCCGGCCTC
GCGTTGTAGAGATGGCCAACCTGCTGAACCTCAGCGAGCGGCAGATCAAGATCTCTC
CTCTCACCACGCGCCTCCTCCTCAGGGTAGAATCCAAGAAGCGCCCAAATTAACACA
CCTTACATCTTTGTAGGTAATTCCCCCCAAATCTTGATTTTTTTTTTCCTCAANTAT
CGGTTTCTTCCACGAAACCTAAACTTTCACAATCCTCTTCCGCNGCCACAAAGAAGG
TGTCACGTGACCCGAAAGCCAAACACCATTG
Thymosin-beta-4
ACAACTCGGTGGTGGCCACTGCGCAGACCAGACTTCGCTCGTACTCGTGCGCCTCGC
TTCGCTTTTCCTCCGCAACCATGTCTGACAAACCCGATATGGCTGAGATCGAGAAAT
TCGATAAGTCGAAACTGAAGAAGACAGAGACGCAAGAGAAAAATCCACTGCCTTCCA
AAGAAACGATTGAACAGGAGAAGCAAGCAGGCGAATCGTAATGAGGCGTGCGCCGCC
AATATGCACTGTACATTCCACAAGCATTGCCTTCTTATTTTACTTCTTTTAGCTGTT
TAACTTTGTAAGATGCAAAGAGGTTGGATCAAGTTTAAATGACTG
TGCTGCCCCTTTCACATCAAAGAACTACTGACAACGAAGGCCGCGCTGCCTTTCCCA
TCTGTCTATCTATCTGGCTGGCAGGGAAGGAAAGAACTTGCATGTTGGTGAAGGAAG
AAGTGGGGTGGAAGAAGTGGGGTGGGACGACAGTGAAATCTAGAGTAAAACCAAGCT
GGCCCAAGTGTCCTGCAGGCTGTAATGCAGTTTAATCAGAGTGCCATTTTTTTTT
Glucocerebrosidase or acc. #: M16328
CCATTAGGCCTATGAATTATAAGATACAGTCACTTTAAAATCCACTGGAAGGCTGAA
GAGTGAGTTAAACCTCTTATAATGAATATACAGTGAAACCAGTAGAGGCATTTTATT
TAGGGTTCCTACAAGAAAGTGCTTAAATAGCATCGACGCCTACATGCTACATCCTGT
TCAGTCTCTGCCTCTGTGATGCAGTTGGCCAGCAAATATCCTCCAAGTCATCATTTG
CATAGTGCTAGGGATAAAATGAGGAGCAATACCAAATGCTATACCTGCCCTTATGGG
TCTTATAGTCCAACGGGAGAAAAAGATATTATACAAATAATCACGGAAAATAAATAG
AAAACGCATCCTTGTTTTTGTTTAGTGGATCCTCTATCCTTCAGAGACTCTGGAACC
CCTGTGGTCTTCTCTTCATCTAATGACCCTGAGGGGATGGAGTTTTCAAGTCCTTCC
AGAGAGGAATGTCCCAAGCCTTTGAGTAGGGTAAGCATCATGGCTGGCAGCCTCACA
GGTTTGCTTCTACTTCAGGCAGTGTCGTGGGCATCAGGTGCCCGCCCCTGCATCCCT
AAAAGCTTCGGCTACAGCTCGGTGGTGTTGTCTGCAATGCCACATACTGTGACTCCT
TTGACCCCCCGACCTTTCCTGCCCTTGGTACCTTCAGCCGCTATGAGAGTACACGCA
GTGGGCGACGGATGGAGCTGAGTATGGGGCCCATCCAGGCTAATCACACGGGCACAG
GCCTGCTACTGACCCTGCACCCAGAACAGAAGTTCCAGAAAGTGAAGGGATTTGGAG
GGGCCATGACAGATGCTGCTGCTCTCAACATCCTTGCCCTGTCACCCCCTGCCCAAA
ATTTGCTACTTAAATCGTACTTCTCTGAAGAAGGAATCGGATATAACATCATCCGGG
TACCCATGGCCAGCTGTGACTTCTCCATCCGCACCTACACCTATGCAGACACCCCTG
ATGATTTCCAGTTGCACAACTTCAGCCTCCCAGAGGAAGATACCAAGCTCAAGATAC
CCCTGATTCACCGAGCCCTGCAGTTGGCCCAGCGTCCCGTTTCACTCCTTGCCAGCC
CCTGGACATCACCCACTTGGCTCAAGACCAATGGAGCGGTGAATGGGAAGGGGTCAC
TCAAGGGACAGCCCGGAGACATCTACCACCAGACCTGGGCCAGATACTTTGTGAAGT
TCCTGGATGCCTATGCTGAGCACAAGTTACAGTTCTGGGCAGTGACAGCTGAAAATG
AGCCTTCTGCTGGGCTGTTGAGTGGATACCCCTTCCAGTGCCTGGGCTTCACCCCTG
AACATCAGCGAGACTTCATTGCCCGTGACCTAGGTCCTACCCTCGCCAACAGTACTC
ACCACAATGTCCGCCTACTCATGCTGGATGACCAACGCTTGCTGCTGCCCCACTGGG
CAAAGGTGGTACTGACAGACCCAGAAGCAGCTAAATATGTTCATGGCATTGCTGTAC
ATTGGTACCTGGACTTTCTGGCTCCAGCCAAAGCCACCCTAGGGGAGACACACCGCC
TGTTCCCCAACACCATGCTCTTTGCCTCAGAGGCCTGTGTGGGCTCCAAGTTCTGGG
AGCAGAGTGTGCGGCTAGGCTCCTGGGATCGAGGGATGCAGTACAGCCACAGCATCA
TCACGAACCTCCTGTACCATGTGGTCGGCTGGACCGACTGGAACCTTGCCCTGAACC
CCGAAGGAGGACCCAATTGGGTGCGTAACTTTGTCGACAGTCCCATCATTGTAGACA
TCACCAAGGACACGTTTTACAAACAGCCCATGTTCTACCACCTTGGCCACTTCAGCA
AGTTCATTCCTGAGGGCTCCCAGAGAGTGGGGCTGGTTGCCAGTCAGAAGAACGACC
TGGACGCAGTGGCACTGATGCATCCCGATGGCTCTGCTGTTGTGGTCGTGCTAAACC
GCTCCTCTAAGGATGTGCCTCTTACCATCAAGGATCCTGCTGTGGGCTTCCTGGAGA
CAATCTCACCTGGCTACTCCATTCACACCTACCTGTGGCATCGCCAGTGATGGAGCA
GATACTCAAGGAGGCACTGGGCTCAGCCTGGGCATTAAAGGGACAGAGTCAGCTCAC
ACGCTGTCTGTGACTAAAGAGGGCACAGCAGGGCCAGTGTGAGCTTACAGCGACGTA
AGCCCAGGGGCAATGGTTTGGGTGACTCACTTTCCCCTCTAGGTGGTGCCCAGGGCT
GGAGGCCCCTAGAAAAAGATCAGTAAGCCCCAGTGTCCCCCCAGCCCCCATGCTTAT
GTGAACATGCGCTGTGTGCTGCTTGCTTTGGAAACTNGCCTGGGTCCAGGCCTAGGG
TGAGCTCACTGTCCGTACAAACACAAGATCAGGGCTGAGGGTAAGGAAAAGAAGAGA
CTAGGAAAGCTGGGCCCAAAACTGGAGACTGTTTGTCTTTCCTAGAGATGCAGAACT
GCGCCCGTGCAGCAGCAGTGTCACCATCAGGGCCGAAGCCTTAAAGCAGCAGCGGGT
GTGCCCAGCCACCCACATGATTCCTATGGCACCAGCCAGGAAAAATGGCAGCTCTTA
AAGGAGAAAATGTTTGAGCCC
PU.1 (Spi-1) or accession # X66079
CCACCATGCTCGCCCTGGAGGCTGCACAGCTCGACGGGCCACACTFCAGCTGTCTGTACC
CAGATGGCGTCTTCTATGACCTGGACAGCTGCAAGCATTCCAGCTACCCTGATTCAGAGG
GGGCTCCTGACTCCCTGTGGGACTGGACTGTGGCCCCACCTGTCCCAGCCACCCCCTATG
AAGCCTTCGACCCGGCAGCAGCCGCTTTTAGCCACCCCCAGGCTGCCCAGCTCTGCTACG
AACCCCCCACCTACAGCCCTGCAGGGAACCTCGAACTGGCCCCCAGCCTGGAGGCCCCG
GGGCCTGGCCTCCCCGCATACCCCACGGAGAACTTCGCTAGCCAGACCCTGGTTCCCCCG
GCATATGCCCCGTACCCCAGCCCTGTGCTATCAGAGGAGGAAGACTTACCGTTGGACAGC
CCTGCCCTGGAGGTCTCGGACAGCGAGTCGGATGAGGCCCT
CGTGGCTGGCCCCGAGGGGAAGGGATCCGAGGCAGGGACTCGCAAGAAGCTGCGCCTGT
ACCAGTTCCTGCTGGGGCTACTGACGCGCGGGGACATGCGTGAGTGCGTGTGGTGGGTG
GAGCCAGGCGCC
GGCGTCTTCCAGTTCTCCTCCAAGCACAAGGAACTCCTGGCGCGCCGCTGGGGCCAGCAG
AAGGGGAACCGCAAGCGCATGACCTACCAGAAGCTGGCGCGCGCCCTCCGAAACTACGC
CAAGACCGGCGAGATCCGCAAGGTCAAGCGCAAGCTCACCTACCAGTTCGACAGCGCGC
TGCTGCCTGCAGTCCGCCGGGCCTGAGCACACCCGAGGCTCCCACCTGCGGA
GCCGCTGGGGGACCTCACGTCCCAGCCAGG
ATCCCCCTGGAAGAAAAAGGGCGTCCCCACACTCTAGGTGATAGGACTTACGCATCCCC
ACCTTTTGGGGTAAGGGGAGTGCTGCCCTGCCATAATCCCCAAGCCCAGCCCGGGCCTGT
CTGGGATTCCCCACTTGTGCCTGGGGTCCCTCTGGGATTTCTTTGTCATGTACAGACTCCC
TGGGATCCTCATGTTTTGGGTGACAGGACCTATGGACCACTATACTCGGGGAGGCAGGGT
AGCAGTGCTTCCAGAGTCCCAAGAGCTTCTCTGGGATTTTCTTGTGATATCTGATTCCCCA
GTGAGGCCTGGGACCTTTTTAAGATCGCTGTGTGTCTGTAAACCCTGAATCTCATCTGGG
GTGGGGGCCCTGCTGGCAACCCTGAGCCCTGTCCAAGGTTCCCTCTTGTCAGATCTGAGA
TTTCCTAGTTATGTCTGGGGCCCTCTGGGAGCTGTTATCATCTCAGATCTCAACGCCCATC
TATGGCTGTGTTGTCACATCTGTCCCCTCATTTTTGAGATCCCCCAAAACTCTGGAACTAT
TCTGCTGCCCCTTTTTATGTGTCTGGAGTCCCCAATCACATCTAGGGCTCCTCC
Mel-18 or accession # : D13969
GAGAGCCCGAACAGGAAGAGGGTACAGCTTTGTGCAGGTCACATGCCCACTGCAGCCCT
CCAGCCTCTGGTCCCCAGAGCGGACTTTGGAAGCTGAACTGCTTTTGAAGCTGGAAGACT
TATGTTATAATTTTACCCTGGGTGGACCAGGGTCGTACAAAAGGGCAACGCTCCCCAGTCC
CCCCACTCCCGACCCCGGAATCATGCATCGGACTACACGGATCAAAATCACAGAGCTGA
ACCCCCACCTCATGTGTGCCCTCTGCGGGGGGTACTTCATCGACGCCACCACTATCGTGG
AGTGCCTGCATTCCTTCTGCAAAACCTGCATCGTGCGCTACCTGGAGACCAACAAATACT
GCCCCATGTGTGACGTGCAGGTCCATAAAACCCGGCCGCTGCTGAGCATCAGGTCTGACA
AAACACTTCAAGACATTGTCTACAAATTGGTCCCTGGGCTTTTTAAAGATGAGATGAAAC
GGCGGCGGGATTTCTATGCAGCGTACCCCCTGACGGAGGTCCCCAACGGCTCCAATGAG
GACCGCGGCGAGGTCTTGGAGCAGGAGAAGGGGGCTCTGAGTGATGATGAGAAAGTCAG
CCTCTCCATCGAATTCTACGAAGGTGCCAGGGACCGGGATGAGAAGAAGGGCCCCCTGG
AGAATGGGGATGGGGACAAAGAGAAAACAGGGGTGCGCTTCCTGCGATGCCCAGCAGC
CATGACCGTCATGCATCTTGCCAAGTTTCTCCGCAACAAGATGGATGTGCCCAGCAAGTA
CAAGGTGGAGGTTCTGTACGAGGACGAGCCACTGAAGGAATACTACACCCTCATGGACA
TCGCCTACATCTACCCCTGGCGGCGGAACGGGCCTCTCCCCCTCAAGTACCGTGTCCAGC
CAGCCTGCAAGCGGCTCACCCTAGCCACGGTGCCCACCCCCTCCGAGGGCACCAACACC
AGCGGGGCGTCCGAGTGTGAGTCAGTCAGCGACAAGGCTCCCAGCCCTGCCACCCTGCC
AGCCACCTCCTCCTCCCTGCCCAGCCCAGCCACCCCATCCCATGGCTCTCCCAGTTCCCAT
GGGCCTCCAGCCACCCACCCTACCTCCCCCACTCCCCCAACGACAGCCA
GTGGGGCCACCACAGCTGCCAACGGGGGTAGCTTGAACTGCCTGCAGACACCATCCTCC
ACCAGCAGGGGGCGCAAGATGACTGTCAACGGCGCTCCCGTGCCCCCCTTAACTTGAGG
CCAGGGACCCTCTCCCTTCTTCCAGCCAAGCCTCTCCACTCCTTCCACTTTTTCTGGGCCC
TTTTTTCCACTTCTTCTACTTTCCCCAGCTCTTCCCACCAAGGGGGTGGGGGGCGGGTTTT
ATAAATAAATATATATATATATGTACATAGGAAAAACCAAATATACATACTTATTTTCTA
TGGACCAACCAGATTAATTTAAATGCCACAGGAAACAAACTTTATGTGTGTGTGTATGTG
TGGAAAATGGTGTTCATTTTTTTTGGGGGGGGTCTTGTGTAATTTGCTGTTTTTGGGGGTG
CCTGGAGATGAACTGGATGGGCCACTGGAGTCTCAATAAAGCTCTGCACCATCCTCGCTG
TTTCCCAAGGCAGGTGGTGTGTTGGGGGCCCCTTCAGACCCAAAGCTTTAGGCATGATTC
CAACTGGCTGCATATAGGAGTCAGTTAGAATTGTTTCTTTCTCTCCCCGTTTCTCTGCC
CATCTTGGCTGCTGTCCTGCCTCTGACCAGTGGCCGCCCCCCGCGTTGTTGAATGTCCAGA
AATTGCTAAGAACAGTGCCTTTTACAAATGCAGTTTATCCCTGGTTCTGAGGAGCAAGTG
CAGGGTGGAGGTGGCACCTGCATCACCTCCTCCTCTTGCAGTGGAAACTTTGTGCAAAGA
ATAGATAGTTCTGCCTCTTTTTTTTTTTTTTCCTGTGTGTGTGGCCTTTGCATCATTTATCTT
GTGGAAAAGAAGATTCAGGCCCTGAGAGGTCTCAGCTCTTGGAGGAGGGCTAAGGCTAA
AGCATTGTGAAGCGCTGCACCCCCACCAACCTTACCCTCACCGGGGAACCCTCACTAGCA
GGACTGGTGGTGGAGTCTCACCTGGGGCCTAGAGTGG
AAGTGGGGGTGGGTTAACCTCACACAAGCACAGATCCCAGACTTTGCCAGAGGCAAACA
GGGAATTCCGCCGATACTGACGGGCTCCAGGAGTCGTCGCCACACTCG
BSK-87-5 - reverse
GGTAATACTTAGAGCATTACAAAGCACTTTCACATTTAAATTTGATTTTGGAAAGTA
TTTTCTTTTTGAGACAGAGTCTCTGTCACCCAGGCTGGAGTGCATGGAGTGCAGTGG
TGCAAACACAGCTCGCTGCACCCTCAACCTCCTGGGCTCAAGCAGTCTTTCCACCTC
GGCCTCCCAAGTTGCTAGGACTATAGGACTACAG
BSK-88-1 forward
TGAGCTTGAACGCTTTCTTAATTGGTGGCTGCTTTTAGGCCTACTATGGGTGTTAAA
TTTTTTACTCTCTCTACAAGGTTTTTTCCTAGTGTCCAAAGAGCTGTTCCTCTTTGG
ACTAACAGTTAAATTTACAAGGGGATTTAGAGGGTTCTGTGGGCAAATTTAAAGTTG
AACTAAGATTCTATCTTGGACAACCAGCTATCACCAGGCTCGGTAGGTTTGTCGCCT
CTACCTATAAATCTTCCCACTATTTTGCTACATAGACGGGTGTGCTCTTTTAGCTGT
TCTTAGGTAGCTCGTCTGGTTTCGGGGGTCTTAGCTTTGGCTCTCCTTGCAAAGTTA
TTTCTAGTTAAT
BSK-88-1- reverse
ATTAACTAGAAATAACTTTGCAAGGAGAGCCAAAGCTAAGACCCCCGAAACCAGACG
AGCTACCTAAGAACAGCTAAAAGAGCACACCCGTCTATGTAGCAAAATAGTGGGAAG
ATTTATAGGTAGAGGCGACAAACCTACCGAGCCTGGTGATAGCTGGTTGTCCAAGAT
AGAATCTTAGTTCAACTTTAAATTTGCCCACAGAACCCTCTAAATCCCCTTGTAAAT
TTAACTGTTAGTCCAAAGAGGAACAGCTCTTTGGACACTAGGAAAAAACCTTGTAGA
GAGAGTAAAAAATTTAACACCCATAGTAGGCCTAAAAGCAGCCACCAATTAAGAAAG
CGTTCAAGCTCA
BSK-88-1-2 - forward
GCCGCCTATTTCCTCCGAAACCCGCGCTGCGGAGCAGCCCAGTGCATAGAGTTCAAC
ACTTCCCCTTGTTGTGGAAAGTAAAGGAGCCTCACTACCACCTTTTTTTCTTTGCGT
TTTCTTACTGCTGGTCCTGGGAGCCTTTTCCTTCGGAGCAGCAGCCCTGTCCGGCAT
CTGTCTTGAGCTCCCAGCAAGGAAAGTCCATCAGCTTGATAATGGAGGAGAACAATG
ACTCCACGGAGAACCCCCAACAAGGCCAAGGGCGGCAGAATGCCATCAAGTGTGGGT
GGCTGAGGAAGCAAGGAGGCTTTGTCAAGACTTGGCATACTCGCTGGTTTGTGCTCA
AGGGGGATCAGCTCTATTATTTCAAAGATGAAGATGAAACCAAGCCCTTGGGTACTA
TTTTTCTGCCTGGAAATAAAGTTTCTGAGCATCCCTGCAATGAAGAGAACCCAGGGA
AGTTCCTTTTTGAAGTAGTTCCAGGTAAGATATTTTCCTAGTCTGATTAAATTATTG
CATCCTGGGTGGTAAAGGTGAANATGGGTCAAACAGGNTTCATTCTTTTTTGAATCA
TGACTGAGACCTTAATTTGAGGCTTGGNTAATGGTGACCCAAATAATGATGCAGGGT
TATTTCTAATCAAATGAATGCCTCCCCACTACTNTGACACATAATATAAATTTATTT
GNCATGAACTCATANTGACCCANNNTGAG
BSK-88-1-2- reverse
GCAAAACCTCCTTGAAGATACAATTTTGTGAGGAAATATGTCAGTGATTCCACTGGG
CAAAGCATTCAACCTATAACCCCTTGTCAAATTTCACATCACAAGAGCGCTGTAAAA
TCAAATTCATCTCCAATAGTCCTGAACAAATACTGTATCATGACTTGTGGTCAACTA
TGGAGTCTCATGGACAAATGAAAATCTANTAGTTATGTGGNCANAGTATGTGTGNGN
GANCGCATTCATTNGNNCTANNATATAANCNTG
BSK-88-2 - forward
TGAGCTTGAACGCTTTCTTAATTGGTGGCTGCTTTTAGGCCTACTATGGGTGTTAAA
TTTTTTACTCTCTCTACAAGGTTTTTTCCTAGTGTCCAAAGAGCTGTTCCTCTTTGG
ACTAACAGTTAAATTTACAAGGGGATTTAGAGGGTTCTGTGGGCAAATTTAAAGTTG
AACTAAGATTCTATCTTGGACAACCAGCTATCACCAGGCTCGGTAGGTTTGTCGCCT
CTACCTATAAATCTTCCCACTATTTTGCTACATAGACGGGTGTGCTCTTTTAGCTGT
TCTTAGGTAGCTCGTCTGGTTTCGGGGGTCTTAGCTTTGGCTCTCCTTGCAAAGTTA
TTTCTAGTTAAT
BSK-88-2 - reverse
ATTAACTAGAAATAACTTTGCAAGGAGAGCCAAAGCTAAGACCCCCGAAACCAGACG
AGCTACCTAAGAACAGCTAAAAGAGCACACCCGTCTATGTAGCAAAATAGTGGGAAG
ATTTATAGCTAGAGGCGACAAACCTACCGAGCCTGGTGATAGCTCGTTGTCCAAGAT
AGAATCTTAGTTCAACTTTAAATTTGCCCACAGAACCCTCTAAATCCCCTTGTACAA
TTTAACTGTTAGTCCAAAGAGGAACAGCTCTTTGGACACTAGGAAAAAACCTTGTAG
AGAGAGTAAAAAATTTAACACCCATAGTAGGCCTAAAAGCAGCCACCAATTAAGAAA
GCGTTCAAGCTCA
BSK-88-3 - forward
GTGTGACTTCACCGAAGACCAGACCGCAGAGTTCAAGGAGGCCTTCCAGCTGTTTGA
CCGAACAGCTGATGGCAAGATCCTGTACAGCCAGTGTGGGGATGTGATGAGGGCCCT
GGGCCACAACCCTACCAACGCCGAGGTGCTCAAGGTCCTGGGGAACCCCAAGAGTGA
TGAGATGAATGTGAAGGTGCTGGACTTTGACCACTTTCTGCCCATGCTGCAGACAGT
GGCCAAGAACAAGGACCAGGGCACCTATGAGGATTATGTCGAAGGACTTCGGGTGTT
TGACAAGGAAGGAAATGGCACCGTCATGGGTGCTGAAATCCGGCATGTTCTTGTCAC
ACTGGGTGAGAAGATGACAGAGGAAGAAGTANAGATGCTGGTGGCAGGGCATCAGGA
CAGCAATGGTTGTATCAACTATGAAGAGCTCGTCCGCATGGTGCTGAATGGCTGAGG
ACCTTCCCAGTCTCCCAAATCCGTGCCTTTCCCTGTGTGAATTTTGTATCTACCTAA
AAGTTTCCCTAGCTTTTTTGTTCANCACTTTCCATTTGTTTTNTTGATGATGTTGCC
GCACATTCACCAATAACTTGTTTTTGGCC
BSK-88-3 - reverse
GGCCCAGAGAGCAAGTTTATTTGGTGAATGCTGACGGCAAACATCATCCAAGAGAGA
CAAGATGGGAAAGTTGCTGAGACAAGAAAGCCTACGGAAACTTTAGGCTAGATACAA
AATTCACACAGGGAAAGGCACGGACTCTGGGGAGACTGGGAAGGTCCTCAGCCATTC
AGCACCATGCGGACGAGCTCTTCATAGTTGATACAACCATTGCTCTCCTCATGCCCT
GCCACCAGCATCTCTACTTCTTCCTCTGTCATCTTCTCACCCAGTGTGACAAGAACA
TGCCGGATTTCAGCACCCATGACGGTGCCATTTCCTTCCTTGTCAAACACCCGAAGT
CCTTCGACATAATCCTCATAGGTGCCCTGGTCCTTGTTCTTGGCCACTGTCTGCAGC
ATGGGCAGAAAGTGCTCAAAGTCCAGCACCTTCACATTCATCTCATCACTCTTGGGG
TTCCCCAGGACCTTGAGCACCTNGGCGTTGGTAGGGTTCTGGCCCAAGGCCCTCATC
ACATCCCCACACTGGCTGNCAGGATCTTGCAT
BSK-1D1 - forward
TTCAGTTTCCTCTCCTAGTAGTACACGAGTCTCCATTGTTTCACATCCTCACCAGTG
CTTTGTATTGTCTGACTTTTAAGATTCTGCTCATCAGACATATGTAAATGACACATA
ACACAGTTTGTTTTCACAGAACAAATGGTTATTTAAATTCTAAACCCAAAGTAATGT
ACAATTACAATAAAAGGCCAGAAGAAAGAGGAGGAAGGAAAAAGATGTGAGAAATAA
AATTGTTATAGTAATTCTTGTTTTCGCTTCCAAGCATAAAATAGTAATTGGAATGTT
TAGTGTGCATGTGTGTATACAATGCAATATGATACAATATAAAAGCAATGCCTCTCT
TTGTTCCATTGGTTGNTTTTTAAATCTATTTTTATAAGTAATAAG
BSK-1E10-9
CTGGAATCTAGATAGTTTTCAGGATGGGGAAGATAGATTCAAAACCACCTAAGGGCA
TTCTGGGTACAAAGCATTGTGCAAGGCTTTGGTGATACAGAGAATAAGGTCTTTTTT
CCCATACTTCCTCATCTGCCAAGGTTATCTCCAATTGTACCTTTCTCTCCAGTTCCA
AGCTTGC
BSK-1L2-1 - forward
CGGTAAATTTTTTACTCTCTCTACAAGGTTTTTTCCTAGTGTCCAAAGAGCTGTTCC
TCTTTGGACTAACAGTTAAATTTACAAGGGGATTTAGAGGGTTCTGTGGGCAAATTT
AAAGTTGAACTAAGATTCTATCTTGGACAACCAGCTATCACCAGGCTCGGTAGGTTT
GTCGCCTCTACCTATAAATCTTCCCACTATTTTGCTACATAGACGGGTGTGCTCTTT
TAGCTGTTCTTAGGTAGCTCGTCTGGTTTCGGGGGTCTTAGCTTTGGCTCTCCTTGC
AAAGTTATTTCTAGTTAATTCATTATGCAGAAGGTATAGGGGTTAGTCCTTGCTATA
TTATGCTTGGTTATAATTTTTCATCTTTCCCTTGCCGAAATTCCG
BSK-1K9-B1 - forward
GTCAGAAAACCACACATGCAGACCCAGAGAGAATGGTTTCTTTGTGGTTTGCACAGG
AAAAACCATCCTTACTAAAAGACAGGGTAGAGTGGGGAGGGTGCAGGAAGGAACTCA
TAATGACCATTTTCCCACATGGAGAAGCAAAGCAGCTTCTTCAGGGCTCAATCAGTT
ATGAAAAAGAATCTCACCCCATTAGATAGCACTCCTGAGCTCAGTGTAGGGTCCCAA
GCCCACCAACCAAGGCTGTCTCCCCAGAACAAATCAGGAAGGCTCCAGTGGTCAGAT
AGAAAGTGACAAACAAAACATGAGTGCATCTAGCCACATCCTCACATTCCACACAAG
AGAACCCATGTGACTAAACAGGAATCCCCTGCTGCCCCAGTTCTAAAAAGGAACTAC
TGACTGCCAGTGCAATTTCTT
BSK-1K9-B1 - reverse
CAAGGAAATTGCACTGTGCAGTCAGTAGTTCCTTTTTAGAACTGGGTGCAGCAGGGG
ATTCCTGTTTAGTCACATGGGTTCTCTTGTGTGGAATGTGAGGATGTGGCTAGATGC
ACTCATGTTTTGTTTGTCACTTTCTATCTGACCACTGGAGCCTTCCTGATTTGTTCT
GGGGAGACAGCCTTGGTTGGTGGGCTTGGGACCCTACACTGAGCTCAGGAGTGCTAT
CTAATGGGGTGAGATTCTTTTTCATAACTGATTGAGCCCTGAAGAAGCTGCTTTGCT
TCTCCATGTGGGAAAATGGNCATTATGAGTTCCTTTCTGCACCCTCCCCACTCTACC
CTGTCTTTTANTAAGGATGGGTTTTNCTGTGCAAACCACAAAGAAACCNTTCTCTCT
GGG
BSK-2A15-A1 - forward
TGCAGCTCGCCTTGCACAACAGGAAAAACAANAACAAGTTAAAATTGAGTCTNTNGC
CAANAGCTTAAAAAATGCTNTGAGGCAAACTGCAAGTGTCACTNTGCAGGCTATTGC
AGCTCAAAATGCTGCGGTCCAGGCTGTCAAT
BSK-2A15-A1 - reverse
GCATTGACAGCCTGGACCGCAGCATTCTGAGCTGCAATAGCCTGCAGAGTGACACTT
GCAGTTTGCCTCAGAGCATCTTCTAAGCTCTTGGCTAGAGACTCAATTTTAACTTGT
TCTTGTTTTTCCTGTTGTGCAAGGCGAGCTGCAT
BSK-2A15-D3 - forward
GCTGGAACAGAATAGCCTGGAACAGGATCTTTCGTTCCATAATATTTTTTAATTAGA
GCAAGTCCTGCTACTGTATCTGTTCCTTTGAAGTTAACCAAGTGAGCAGATGCTCCT
ATGCCAGCAGTCTCTTGGGAAGAGACTCCTCTGTAGCCAAAATCATGTAACTTGTAT
TCCAGACCATCTAAGTTACCAGAAGTTTCTAACAAATATTTGGCCAATATTTTCTTC
TGCTCTCTAGAATTTGTGGCCACTGTCATTGGATACCAGGACTGAACAAGAATAGTC
TCAATCCAATTTGTAAGCCAGTAACACTCTGGATCTGTGTTTTCCACCGTGAAGAAA
CATTTCCTCTGGGAATGACAAANCCCTCANGAACAGCTTTTATTTCTATTGGAAGAT
GCCCATCATACTTCTCAAGAATGGAGTTCCTCCCTTTTCATTAAAGACATCATCTTG
GAAATGTTCTTTGTAGACATCTTTGGCTTCCTGGATTTCTCTTTGGGTACTACTTTA
CCTTTTAAGNACTTATTAANAAAGNACTGNACCCATAAAAACTGGNNCTCATATTTA
NCTTCCTTAATTGGAGGNTNTGNTTNTTTTACGGNTTCAAAGANGAAAAAATTTCTT
GNGTGGGGGGANTTG
BSK-2A15-D3 - reverse
GCCGCGCCAGGGAGCTCGCGGCGCGCGGCCCCTGTCCTCCGGCCCGAGATGAATCCT
GCGGCAGAAGCCGAGTTCAACATCCTCCTGGCCACCGACTCCTACAAGGTTACTCAC
TATAAACAATATCCACCCAACACAAGCAAAGTTTATTCCTACTTTGAATGCCGTGAA
AAGAAGACAGAAAACTCCAAATTAAGGAAGGTGAAATATGAGGAAACAGTATTTTAT
GGGTTGCAGTACATTCTTAATAAGTACTTAAAAGGTAAAGTAGTAACCAAAGAGAAA
ATCCAGGAAGCCAAAGATGTCTACAAAGAACATTTCCAAGATGATGTCTTTAATGAA
AAGGGATGGAACTACATTCTTGAGAAGTATGATGGGCATCTTCCAATAAAAATAAAA
ACTGTTCCTGAGGGCTTTGTCATTTCCANAGCAAATGTTTCTTNNCGGGGGAAAACA
CAGATCCNAAGGGGNACTGGNTTACAAATTGGATTGAGANTATTCTTGGTNANNCCT
CGGATCCAATCCAAGGGCGCCCAAATT
BSK-2A3-A2 - forward
CACGAGCGCACGTGTTAGGACCCGAAAGATGGTGAACTATGCCTGGGCAGGGCGAAG
CCAGAGGAAACTCTGGTGGAGGTCCGTAGCGGTCCTGACGTCCAAATCGGTCGTCCG
ACCTGGGTATAGCGGCGAAAGACTAATCGAACCATCTAGTAGCTGGTTCCCTCCGAA
GTTTCCCTCAGGATAGCTGGCGCTCTCGCAGACCCGACGCACCCCCGCCACGCAGTT
TTATCCGGTAAAGCGAATGATTAGAGGTCTTGGGGCCGAAACGATCTCAACCTATTC
TCAAACTTTAAATGGTAANAAGCCCGGCTCGCTTGGCGTGGAGCCGGGCGTGGAATG
CNAGTGCCTAATGGGCCACTTTTGGTAANCAAAACTGGCGCTGCGGGATGAACCCAA
CGCCCGGTTAANGGGCCCNATGCCGACCTCATNANACCCCANAAAANGNGTTGGNTG
ATAC
BSK-2A3-A2 - reverse
TATCAACCAACACCTTTTCTGGGGTCTGATGAGCGTCGGCATCGGGCGCCTTAACCC
GGCGTTCGGTTCATCCCGCAGCGCCAGTTCTGCTTACCAAAAGTGGCCCACTAGGCA
CTCGCATTCCACGCCCCGCTCCACGCCAGCGAGCCGGGCTTCTTACCCATTTAAAGT
TTGAGAATAGGTTGAGATCGTTTCGGCCCCAAGACCTCTAATCATTCGCTTTACCGG
ATAAAACTGCGTCGCGGGGGTGCGTCGGGTCTGCGAGAGCGCCAGCTATCCTGAGGG
AAACTTCGGAGGGAACCAGCTACTANATGGTTCGATTAAGTCTTTCCCCCCTATACC
CAGGTCGGACGACCGATTTGCACGTNAGGACCGCTACGGACCTCCCCANAGTTCCTN
TGGNTTNGCCCTGCCAGGCTANTNACCATNTTTGGGNCTAAACGNGCGCTCGGCCGG
AATTCNCCGANCTGANGGCTCCNGAATNNNNCCCCCATCCCAGC
BSK-2A3-B3 - forward
GCCCCCCCTAACCGGCTTTTTGCCCAAACGGGCCATTATCGAAGAATTCACAAAAAA
CAATAGCCTCATCATCCCCACATCATAGCCACCATCACCCTCCTTAACCTCTACTTC
TACCTACGCCTAATCTACTCCACCTCAATCACACTACTCCCCATATCTAACAACGTA
AAAATAAAATGACAGTTTGAACATACAAAACCCACCCCATTCCTCCCCACACTCATC
CCCCTTACCACGCTACTCCTACCTATCTCCCCTTTTAT
BSK-2A3-B3 - reverse
ATAAAAGGGGAGATAGGTAGGAGTAGCGTGGTAAGGGCGATGAGTGTGGGCAGGAAT
GGGGTGGCTTTTGTATGTTCAAACTGTCATTTTATTTTTACGTTGTTAGATATGGGG
AGTAGTCTGATTGAGGTGGAGTAGATTAGGCGTAGGTAGAAGTAGAGGTTAAGGAGG
CTGATGGTGGCTATGATGTGGGGATGATGAGGCTATTGNTTTTTGTGAATTNTTNNA
TAATGCCCCGTTTGGGCAAAAAGCCGGNTANCGGGGGCCG
BSK-2E14-D4 - forward
CGGGACTTTACCCCATCATTTGCAGAACCAGTATCAATATTCGTAAGGTAACTGCTC
TTAAAACTCANAATCATCCTAACTGGATGTAAAAACTTTTTCCCAGAAAATGTTGGC
GTGCACTCACAAAACCCTCTTACTTCATTTTCTCCATATAATGACTCTATGGGGGGA
GGGCGCCAGGTGTCCTCATTCTCATTTGAAATTTGAATTCCAATCTTGTTAGAATGT
AGCCCAACTCCTTTCCTTTCTCAGGAAAGTGGCGACAGTTCTCAGGTCTGCCTCCAC
ATTACCATCACCTGGGGGATCTAAAACTACTCAGGCCTGGGTTCCACCTTCAGCCAA
CGAAATCTGAATCTTTANGGGTGGCTGATTTCGCTGTTCTGTAAATGAAGTTTTAAT
GGTCACAGCCCGTCTGACCGTTTGCATA
BSK-2E14-D4 - reverse
TATGCAAACGGTCAGACGTGGCTGTGACCATTAAAACTTCATTTACAGAACAGCGAT
ATCAGCCACCCCTAAAGATTCAGATTTCGTTGGCTGAAGGTGGAACCCAGGCCTGAG
TAGTTTTAGATCCCCAGGTGATGGTAATGTGGAGGCAGACCTGAGAACTGTCGGCCA
CTTTCCTGAGAAAGGAAAGGAGTTGGGCTACATTCTAACAAGATTGGAATTCAAATT
TCAAATGAGAATGAGCACACCTGGCCCCCTCCCCCCATAGAGTCATTATATGGAGAA
AATGAAGTAAGAGGGTTTTGTGAGTGCACCCCAACATTTTNTGGGAAAAAGGTTTTA
CATCCAGTTAGGATGATTCTGAGTTTTAAGANCANTTACCTTACCAATATTGATACT
GGGTCTGCNAANGATGCGGGAAAATCCCCCGGNATTCACTGAGCGCCNGGCGGTACC
ATTACAANTGGNTNGGGGGNAAAAATAATAATNACCGGCAGGCATGTTAAGNCCAAA
TTTTNGAAATTCTTNCACTGGGGGGCGGTTNACTTCTTTTNAAGGGCCAATNNCCCN
TATGAGGNGNNTANAANTNCTGGCCNNGNTTTCANNNNNNACNGGAAAACTGGGGTT
CCCAATTAANTNNTTTNNNNNANNCCCTTTTCCCTGGGNANANNAAAAAGGCCNNNC
CANTNCCNTTCNANANTNNCNNCTANNGGGAANGGNNCCCCNTNNNGNNCNTAANCN
GGGGGGGGGGGTNCCC
BSK-2F6-D3 - forward
CAACAACACATCATCAGTAGGGTAAAACTAACCTCTCTCACGACGGTCTAAACCCAG
CTCACGTTCCCTATTAGTGGGTGAACAATCCAACGCTTGGTGAATTCTGCTTCACAA
TGATAGGAAGAGCCGACATCGAAGGATCAAAAAGCCGACGTCGCTATGAACGCTTGG
CCGCCACAAGCCAGTTATCCCTTGTGGTAACTTTTCTGACACCTCCTGCTTAAAACC
CAAAAGGTCAGAAGGATCGTGAGGCCCCGCTTTCATGGGCAGTAGGCAGATTCGTCC
BSK-2F6-D3 - reverse
GGACGAATCTGCCTACTGCCCATGAAAGCGGGGCCTCACGATCCTTCTGACCTTTTG
GCTTTTAAGCAGGAGGTGTCAGAAAAGTTACCACAGGGATAACTGGCTTGTGGCGGC
CAAGCGTTCATAGCGACGTCCCTTTTTGATCCTTCGATGTCGGCTCTTCCTATCATT
GTGAAGCAGAATTCACCAAGCGTTGGATTGTTCACCCACTAATAGGGAACGTGAGCT
GGGTTTAGACCGTCGTGAGACAGGTTAGTTTTACCCTACTGATGATGTGTTGTTG
BSK-2G3-A3 - forward
TTTTTTTTTTTTTTTTGAGACAGAGTCTCACTCTGTCACCCAGGCTGGGGTGCAATG
GCATGCTCTCAGCTCACTGCAACCTCTGCCTCCTGGGTTCAAGCGATTCTCCTGCCT
CAACCTCCCGAGTGACTGGGATTACAGGCATGCACCACCATACCCAGCTAATTTTTG
TATTTTTAGTANAGATGGGGTTTCACCATGTTGGTGAGGCTGATCTTAAACTCCTGA
CCTCAGGTGATCTGCCTGCCTTGGCTTCCCAAAGCACTGGGATTACAGGTGTGAGCC
ACCATGCCTGGCCTATTTTGAAAAAAATTTGAAGTCAAAATAATAGTACAATAAATA
CCTGTGAACCCTTCAGCTATATTTACCAATTGTTAATATTTTACCATGTTTGCTTCA
TCTCTCTACATATGTATTCATATGTAATTTTTTTTATTTTTGCCAAAACATTTGAAA
ATTAAACATCTGGATACTTTGCCATTAAANCCTTCAACATGAATCTCCTAAAAAATA
ANAACAGCTTNTATCCCCATACCNTTATCACATCCCAGAAAATTACCCCCNTACATT
NAATGACTACTNCNGCCCTATCAAATTNTTTGATATCCAAACTTTCTTTGGGGGGNT
TTTTTCCCNNCCCNAATCANTCANGNCCNCCATTGNNTTTAATGGGNAGNTNCTNNA
NNNAAAATATCCNCCTTTTTTCTTTNTGANTTGNCTTTTAAAAAACCANTNANANCC
TGGGNGNTNCCAACNGNNTTNTGG
BSK-2G3-A3 - reverse
CGGCTTTGTGGAAGACAGTTTTTCCGTGAACAGGGGTTGGAGGTGGTGGTGGGAGGG
ATGGTTTTGGGATGAAACTGTTCCACCTCAGATCATTAGGTATTAGATTCTCATAAA
GAGCACACAGCCTANATCCCTCACATGTGCAGTTCCTATGAGAATCTAATGCCACAG
TTCACCCGCCACTCACCGCTGTGAGTGGCCTTGTTCCTAACAGACCATGGACCANTA
CTGGCCCGTGGCCCANGGGTTAGGGACCCCTGATCTAACACATANATCTAATGAAGA
AACAGGTTCCATGTGTTAAAAATCTGTGGTTGAAACTGACATTATATTCCTCCTGAT
TTGATACCATGGGGAATACANAACATGACCTATGTGGTACTCCTACCAAAAACGTTT
NACTTGAATCTAACCATGANCAAACATCCANACAANTACAGCTTGTGAGAGCCTCNC
ANGCTGNTACTTGGATTTTTTAAAANNGNNNTGNNTNAAAGGAAAAAAGGNNGGGNT
ANTNTNNATTAANGAACTTNCNNTNAANGCNNGNGNGGNCTTGNTGAANNTNGATGG
GAAAAAANCNCCCC
BSK-2G3-C5 - forward
AGCACACTGGCGGCCGTTACTAGTGGATCCGAGCTCGGTACCAAGCTTGATGCATAG
CTTGAGTATTCTATAGTGTCACCTAAATAGCTTGGCGTAATCATGGTCATAGCTGTT
TCCTGTGTGAAATTGTTATCCGCTCACAATTCCACACAACATACGAGCCGGAAGCAT
AAAGTGTAAAGCCTGGGGTGCCTAATGAGTGAGCTAACTCACATTAATTGCGTTGCG
CTCACTGCCCGCTTTCCANTCGGGAAACCTGTCNTGCCANCTGCATTAATNAATCNG
NCAACNCNCGGGGAGAGGCGGTTTNCNTATTNGGCGCTCTTNCNCTTCTCNNTCACT
GACTCNTGNCTCNGNCNNTNNNNTNNNGNNANCGGATANNTNACTTCAAANGCGGNA
TACGNTATCCANAATNANGGGATAACNCNNNAAAAAACAT
BSK-2G3-C5- reverse
AGCACACTGGCGGCCGTTACTAGTGGATCCGAGCTCGGTACCAAGCTTGATGCATAG
CTTGAGTATTCTATAGTGTCACCTAAATAGCTTGGCGTAATCATGGTCATAGCTGTT
TCCTGTGTGAAATTGTTATCCGCTCACAATTCCACACAACATACGAGCCGGAAGCAT
AAAGTGTAAAGCCTGGGGTGCCTAATGAGTGAGCTAACTCACATTAATTGCGTTGCG
CTCACTGCCCGCTTTCCANTCGGGAAACCTGTCNTGCCANCTGCATTAATNAATCNG
NCAACNCNCGGGGAGAGGCGGTTTNCNTATTNGGCGCTCTTNCNCTTCTC
BSK-2K15-A1 - forward
CTAAACTTAGGGCAACCCCAAGCGCTTGAACCTATACCACCCCACTTTCCTGAGCTC
TGTAAAGAGCATGAAGTTTTCCCACTGACCCCATACACTGAGGTGCCATCACACTGC
ACATTTCCTTCCGGAGAACAAGCACGTACTCAGGTGGAGATAGAACTGTCTTTTTAC
TTAATAGAAAATGATGTGGCAGCTTTAAGAGGAGCGCGTCGGTCTGGGGCTGGTGGC
TTGGGTCACGTGACACCGGTGGTCTCGTTTGCGCCTCTTGATGTCGCGGCGGCGCCC
TGAGGACGGATTGGGCAAGGCTGGTCCCTGTGTGATGAGACATCACCCTCCCAGGAG
CAAGGCGGAAGTCTGGAGGACCTTANGGGCGGANGCGGGAGAAGCNAACTCCGATGA
ATGGTCTCGGCAGGCTCTTCGGGAAAGGGTGAGCCANGGTGGGACTGGCCAGCCAGG
AAGCCTGCTGGTGCAGGGGAAANAAGANANCCCGCGAGATTNGGCCGGACCCTTCCC
GGCNGGGGAAGAAAATCAGGAGAACAGGCTGACTGGAAAANCCCGCGGNCCATGGNG
GACAAGGGTATTNCCGGGGCCAAAAGGNCACCATGTNGGNGGAATTCCNCTGACNCC
GGCGTTACATTAACANTNGGNTGGGGGNAAAANAAAATAACCGGNNGGCCTGTNAGC
CAAATTCACNNCTGGNGGGCGTNTTTGGNTCCACNNGNCCNACTTGANNNNANTTNN
GNTTTTTTNGGNNCCNAAAANTGGGGA
BSK-2K15-A1 - reverse
CCGACATGGTGTGCCTTTTGGCACCGGCGATGAGCCTTGCTCCGCCATCGGCCGCCG
GGGTTTTCCAGTCAGCCTGTCTCCTGATTCTCTTCCCCTGCCCGGCGCAGCGGTCCG
GCCGAATCTCGCCGGGGTCTCCTCTTCCCCTGCACCAGCCAGCGCCTCCTGGCTGGC
CAGTCCCACCCTGGCTCACCCTTCCCGAAGAGCCTGCCGAGACCACTCATCGCGAGC
TCGCCTCTCCCGCCTCCGCCCCTCAGCGTCCTCCAGACTTCCGCCTTGCTCCTGGGA
GGGTGATGTCTCATCACACAGGGACCAGCCTTGCCCAATCCGTCCTCAGGGCGCCGC
CGGACATCAAGAGGCGCAAACNANACACCGGTGTCACGTGAACCAAGCCACCANCCC
ANAACGANCGCTTCTCTTAAAGCTGGCCCATTATTTTTATTAANTAAAAANACAGNT
NTATTTTCACTGANTACNTGCTTGTTNTCCGAAGGAAAGGGC
BSK-2K15-C1 - forward
GATGGCTTATATAACCAGAAGCCAAATATTTGTGTTCCAAAAATTATTTTACTTAGA
ACAATTCATTTAGATTCACTTCAATGTGAAGTATGTGAAAAGCTTAATTGCTGACCA
GAGTGAATTTTCCAACAATAAGAAATGCATGGCTGATTGGCTCAAATGATTCTATTC
TTCAGCCCTTACTGAAGTACTTAGTGCATACCACCTATGTAATTTTATTCCCCCCTT
ATAGAGATGGGGTTTCACCATGCTGCCCAGGCGGGTCTCAAACTCCTAGGTACAAGT
GATCCACCCACTTCGGCCCGCCAAAGGGCCGGGATTACTGGCATGAGCCACCAAGCC
CAGCCTGGTTATGTATTTATTCGGTATCATAGGGGCTACAGCACAAATCAAAACCAT
AGTATCAGTGACCTCCAATCTAATTCCCG
BSK-2K15-C1 - reverse
GGAATTAGATTGGAGGTCACTGATACTATGGTTTTGATTTGTGCTGTAGCCCCTATG
ATACCGAATAAATACATAACCAGGCTGGGCTTGGTGGCTCATGCCAGTAATCCCGGC
CCTTTGGCGGGCCGAAGTGGGTGGATCACTTCTACCTAGGAGTTTGAGACCCGCCTG
GGCAGCATGGTGAAACCCCATCTCTATAAGGGGGGAATAAAATTACATAGGTGGTAT
GCACTAAGTACTTCAGTAAGGGCTGAAGAATAGAATCATTTGAGCCAATCAGCCATG
CATTTCTTATTGTTGGAAAATTCACTCTGGTCAGCAATTAAGCTTTTCACATACTTC
ACATTGAAGTGAATCTAAATGAATTGTTCTAAGTAAAATAATTTTTGGAACACAAAT
ATTTGGCTTCTGGNTATATAANCCATC
BSK-2K15-D1- forward
GAATTGCTTGGACCTGGGAGGCATAGGTTGTGGTAAGCCGAGATTGCGCCATCGCAC
TCCAGCCTGGCCAACAAGAGTGAAACTCCGTCTCAAACAAACAAACAAAAAAGACAC
AAAAGTAAAGGACTTCTTGACCTCTGGTTGAAAGAGTAGCGCATGGGGGGTGTTTCT
GGCAAACAAACCTTCCCAACAACGTCAGAACTGTGTTCACAAATGCTAACCTGTCGG
CCTGGTTATAGAACATCCTCTTCCCTCAGGGGTATCTGGCAGAGGCAGGTACCCGTG
GAATGGTGCAGGTGGTGCCCATGCTCTAGTGTATGCCAAGAGTTCCTACTTTTACAA
AGTAGCCACTTTAAAAAAATGTTGGTACTGGCCAACATTCCTTTCATGCACCCAGGA
GGGCAGCAGGTACCTGGGATCCAAGGATGGATGGCCAGGGCAGGTGGCTGAAAAATG
GGGGTGGGTCAAGAAGGATGTANCTCCTGGGGTGGCGCCCAACAAAAAAAATTANGG
GTAGGGNGGGNGCTATGGNTGGAATGNTTATCCCCCCCAAACTNANNTTNAANGGAA
BSK-2L13-A2 - forward
TGGCCAATGCTCTCTCTGTGAAACTTCAAACTTCAAATGAGGCCCACCTTACATGGG
TCACCATGTGCATGGAAAGAATGTATTTACACTCAGGTACATTCTCGTGGGAAACTG
GAAACCAGCCGGCGGCATCTTGGTGTGACTGCATGCACAATGCATGCGTGTCCTTAA
AGCATTTAATGTTAATGTTTGTATGTGTGAATGCAAAGGAATTTTAATGATATCATG
GCCACATCGAGGTCACTAACCTTGGGAAGTAACATGATCACAAGAATTTTGTATGTG
CTGAGTGACTATTACAGTAACGATTGCAGTGTATAATTGAAGTAGTCCGGCATAATT
TCAAGGGCCCAGACTCCGTGGAAAGAGTTTCCTGACTGAGTCCACGTCCATTCACCA
AGGAAGGCAGGCAGTGGCCTTGCANAAATCCCTCACAATGATGNTGGGCATCCCATC
TACCTTGGTTTTTAGGGCTGGCATAATAATGCCNGNCTATTCANTTTTAAGACAGAT
ATATTTTACNNATAAACCCTGGNGGGGCANAAAANCCCCCTGGNTTCTAACTCTAAC
CTGGGCTCTTNCCTTACTGGGCCCTGGGGGGNTGNTCCTATTCNATNAAAAANCCNC
CANCNGACGGCTCNAGAATNNNNCNCCATCCAANCNAATTCA
BSK-2L13-A2 - reverse
TTCATAGGAATAGGGAACAAACACCACAGTGGCACANTNATGGGAAGGAGCCCAGGC
TAGGAGTTAGGAGACAGTGGCGGCTTCTCTGACACCACCAGGGCTCTCATCTGTAAA
ATGATATCTGTCTTAAAACTGAATGAGACCTGGCATTATTATGGCCAGCCCTGAAAA
CCAAGGTAGATGGGATGCACAACATCATTGTGAGGGATTTCTGCAAGGCCACTGCCT
GCCTTCCTTGGTGAATGGACGTGGACTCAGTCAGGAAACTCTTTCCACGGAGTCTGG
GCCCTTGAAATTATGCCGGACTACTTCAATTATACACTGCAATCGTTACTGTAATAG
TCACTCAGCACATACAAAATTCTTGGGATCATGTTACTTCCAAGGGTAGTGACCTCN
ATGTGGCCATGATATCATTAAAATTCCTTTGCNTTCCCCCTNCCAACATTAACATTA
AATGCTTTAAGGACCCCCTGCNTTTGGCATGCANCACCCAANANGCCGCCCCTGGNT
TCCATTTCCCCCANAAGGACCTGAANGGAAATACTTCTTTCCTCCCATGGGGACCCT
GNANGGGGGCCCANTTNAANTTGAANTTNCAAAAAACATTGGCNCGGAATCCNCTGA
CCCCGGGNGTTNCTTACAANTGGGNNGGGGGNAAAANAAAANAACCGGCNGGCCTGN
NANNCCAATTTNNAAAANCTNNACTGGGGGCGTTG
BSK-2L13-B5 - forward
CAAAGATAAGACCCCCGAAACCAGACGAGCTACCTAAGAACAGCTAAAAGAGCACAC
CCGTCTATGTAGCAAAATAGTGGGAAGATTTATAGGTAGAGGCGACAAACCTACCGA
GCCTGGTGATAGCTGGTTGTCCAAGATAGAATCTTAGTTCAACTTTAAATTTGCCCA
CAGAACCCTCTAAATCCCCTTGTAAATTTAACTGTTAGTCCAAAGAGGAACAGCTCT
TTGGACACTAGGAAAAAACCTTGTAGAGAGAGTAAAAAATTTAACACCCATAGTAGG
CCTAAAAGCAGCCACCAATTAAGAAAGCGTTCAAGCTCAACACCCACTACCTAAAAA
ATCCCAAACATATAACTGAACTCCTCACACCCAATTGGACCAATCTATCACCCTATA
GAAAAACTAATGGTAGTATAAAGTAACATGAAAACATTCTTCTNCGCATAAGCCTGC
GTCAGATTAAAACCTTGAACTGACATTAACAGCCCAATATCTACAATCAACCACAAG
TCATTATTACCCTACTGNNNANCCACCANGCATGCTCNTAAGGAAAGGTTAAAAAAG
TAAAAGGACTCGGNAATNTTACCCGCTGTTTCCAAAAAATTACCTTACNTCNCCNTN
TTAAGGCCCCCTGNCCATGACCATGTTTAAGGCCGNGGNCCCTACCGGCAAAGGGGG
ANAATACTTTTCTTANTAGGGCCCNTTAANGNTCCCCANGGTNANTTTTTTATTTTA
CANNNAATNACTNCCNGAAAGGGGNTNAACNNAANAAAAAAACNT
BSK-2L13-B5 - reverse
GGTTGGGTTCTGCTCCGAGGTCGCCCCAACCGAAATTTTTAATGCAGGTTTGGTAGT
TTAGGACCTGTGGGTTTGTTAGGTACTGTTTGCATTAATAAATTAAAGCCCCATAGG
GTCTTCTCGTCTTGCTGTGTCATGCCCGCCTCTTCACGGGCAGGTCAATTTCACTGG
TTAAAAGTAAGAGACAGCTGAACCCTCGTGGAGCCATTCATACAGGTCCCTAATTAA
GGAACAAGTGATTATGCTACCTTTGCACGGTTAGGGTACCGCGGCCGTTAAACATGT
GTCACTGGGCAGGCGGNGCCTCTAATACTGGNGATGCTAGAGGNGATGTTTTTGGTA
AACAGGCGGGGNAANATTGCCGAGNTCCTTTTACTTTTTTTAACCTTTNCTTATNAA
CATGCCTGTGTTGGGTTGACAGNGAGGGNAATAATGACTNGTGGNTGATGNAAAAAT
TGGGCTGTNATTG
BSK-1B6-A3 - forward
GGGTACCAAATTTCTTTATTTGAAGGAATGGTACAAATCAAAGAACTTAAGTGGATG
TTTTGGTACAACTTATAGAAAAGGTAAAGGAAACCCCAACATGCATGCACTGCCTTG
GTGACCAGGGAAGTCACCCCACGGCTATGGGGAAATTAGCCCGAGGCTTANCTTTCA
TTATCACTGTCTCCCAGGGTGTGCTTGTCAAAGAGATATTCCGCCAAGCCAGATTCG
GGCGCTCCCATCTTGCGCAAGTTGGTCACGTGGTCACCCAATTCTTTGATGGCTTTC
ACCTGCTCATTCAGGTAATGTGTCTCAATGAAGTCACACAAATGGGGGTCATTTTTG
TCAAGTGGCCAGTTTGTGCAGTTCCAGTAGTGACTGATTCACATTTTTTTCCAAATG
TAATGCACACTCCATTGCATTCACCCGNTCTCCCANTCATNACAANCTGGNTTTTGA
TATCCTGAANGAAAAATCGGCCCCTCNTTGGTCTTGANCTTCATCANTTTNTAACAT
GTTCCTTTCCTATGAAATTGGGGAAAAAAGTATTTGCAAATTNTNAAANCCCATTAT
NNCGGNCAAANANTAANAAATGGNCAGGNAACCTAGGNGGAATCCACTTANCCCGGC
NTCCATACCANTGGGCNGNNGCAAAAAAAAATAACCGGCNGGCCTTNAACCAATTCN
CCCTGGNGCCNTCTNNGGATCCACCGGCCAAC
BSK-1B6-A3 - reverse
CCTACGTTTACCTGTCCATGTCTTACTACTTTGACCGCGATGATGTGGCTTTGAAGA
ACTTTGCCAAATACTTTCTTCACCAATCTCATGAGGAGAGGGAACATGCTGAGAAAC
TGATGAAGCTGCAGAACCAACGAGGTGGCCGAATCTTCCTTCAGGATATCAAGAAAC
CAGACTGTGATGACTGGGAGAGCGGGCTGAATGCAATGGAGTGTGCATTACATTTGG
AAAAAAATGTGAATCAGTCACTACTGGAACTGCACAAACTGGCCACTGACAAAAATG
ACCCCCATTTGTGTGACTTCATTGAGACACATTACCTGAATGAGCAGGTGAAAGCCA
TCAAAGAATTGGGTGACCACGTGACCAACTTGCGCAAGATGGGAGCGCCCGAATCTG
GCTTGGCGCAATATCTCTTTGACAAGCACACCCTGGGAGACAGTGATAATGAAAGCT
AAGCCTCGGCTAATTTCCCATACCGTGGGGTGACTTCCTTGGCCCAAGGCAGTGCAT
GCATGTTGGGGTTCCTTACCTTTCTATAATTGGACCAAAACATCCCTTAAGTCTTTG
ATTGNCCATTCTTNAATAAAAAATTTGGACCC
BSK-1C1-2 - forward
GGCTAACAATCTCCAGAAGGTTCATTCAGGCCCATGCAAATCAGTGCCGGAGCCTAG
AGACAGCACAGCCTAGAGCTAGAGGTCAGGCAGGGCTGAGCTGAGTCACCCACTATT
CAGACCTCCCTCTTAGAGCCTCAGCTACTGGATGGTGGTCATTAAGTTATCATTTAA
ACTACAGACGCAGGCTGGGTACGGTGACTCAACCCTATAGCCCCAGCACTTTGGGAG
GCCAAGATGGGAGGATCACTTGAGGTCGGGAGTTCAACACCAGCCTGGCCAACATGA
TGAAACCCCGTCTCTACTAAAAATACAAAAACTAGCTGGGTGTGGGGCGNGCACATC
TTTAATCCCAGCTTCTCANGANGCTGANGCAGGAGGATCACTTAAACCCANNAAGTG
GANGCTGCANGGAGCCCANATCGCACACTTNACTCCACCTGGGTGACAGAATGAGAC
TCATNTTCNAANGAAACCANCNNCCNNTNNCTCNNTGCCNNNNGTANCTNTTACCNA
TCCTTNCCAAGGACCCCACCTTACCATACTTGNTACTAGGNGGCNCCTGAATTTCCN
AAANCNNTCTTAAGGGGGCCTCAAGTTTANNGGCCNTTNCTT
BSK-1C1-2 - reverse
GTGATCTCGGCTCACTGCAACCTCTGCCTCCTGGGTTCAAGCGATTCTTGTGCCTCA
ACCTCCCGAGTAGCTGGCATTACAGGAGCCCGCCCACCATGCCTAGCTAATTTTTGT
ATTTTTAGTAGAGACAGGGTTTCACCATGTTGGCCAGGTTGGTCTGGAACTCCTGAC
CTTGTGATCTGCCTGCCTTCGCCTCCCAAAGTGCTGGAATTACAGGTGTGAGTCACC
GCGCCCAAGTATAGGCCACTTTTAAGAATTACTCANAGTTAGCTTATAAGAGGCGAA
TCAGTGGAGTCCTCCAGTTTGGTTCACACATAATTATTAGGTTGAACCATATAAAGT
TACTGTTTTTGGTCCTGTGAATATTAATATTTATATATGGGTCCAATCTGATATGTT
CCANAAAATACACACTTAANTAAAGNTTGGAAAACCAAATCATANACTTACATACTG
NAAGGCGGGGTATTTGAAACTGGGATGGAAAATCAATTTAATGAGNTATGANCTGCN
TTAAAAAAATGGGANAANATCANANTTGGTGGNANNATTGNAAAAAACCAAATTGCT
GGGGAAGATTGGCATTTNANTNTTNTNNCNCCCNGNGGGGGGGGNNGGGGGNACNAA
ANGNNANAAAGAA
BSK-1D8-2A - forward
CAGTTCCACCCGGGCAGGCAGTCGGGGGATGAGGGGCCGTCTAGCGTCCGCACGCGT
TCACTCCCAAGGAAGGTGTGTGGGCACGGTGAGGAGTGGGAAGAAACAGAATAGGAA
AGTGGCCTGACACGGGGATTCTAAGCAGGTCAAAAGTTATGTTGCCTTGTAGGATGG
GAAGAGAAAAATAAAAATTTGATTTGTTGTTTAAAGTGATGGGGTTCTGGGGATATT
TTTCCTTTTTAATTTTAATATCTTTGGATTAANTTTTTTTTCTCTTTTTTTCAACGG
AGTCTCACTCTCTCACCCTGGGTGGAGTGCANNGGNACNATCTNGGNTNANTGNAAC
CTNCACTTTCTCGGNTCAAGNGANTCTTCTGGCTNANCCTCCNAANANTNGGAATAC
AGGCCCTGCNCCANGCCTGGNTAATTTTGGNTTTTAANAAANCGGAATTCCNCNNNC
TNNNNGCTNNAGA
BSK-1D8-2A - reverse
CTTTACTAAAAATACAAAAATTAGCCAGGCATGGTGGCAGGTGCCTGTAATCCCAGC
TATTCGGGAGGCTGAGGCAGGAGAATCACTTGAACCCAGGAAGTGGAGGTTGCAGTG
AGCCGAGATCGTACCACTGCACTCCACCCAGGGTGACAGAGTGAGACTCCGTTGAAA
AAAAGAGAAAAAAAAATTAATACAAAGATATTAAAATTAAAAAGGAAAAATATCCCC
AGAACCCCATCACTTAAACAACAAATCAAATTTTTATTTTTCTCTTCCCATCCNACA
AGGCAACATAACTCTTACCTGCTTANAATCCCCGTGTCANGCCACTTTCCTATTCTG
TTTCTTNCCACTCCTCANCGNGCCCACACACCTTTCTTGGGAGTGAACGCGTCCCGA
CCTANACGGCCCTCATCCCCGACTGGCTGCCCGGGTGGAACTGGGGAATTCCACTTA
ACGCCGGCGNTCCATTACCAANTGATCTTGGGGCAAAAATAATAATAACCGGCNGGC
CTGTNAAGCCCAANTNTNNAAANTTCTTNNACTTGGNGGCGNTNAGCAGCNTTTNAC
GGCCAATNNCCTATNNGGGNNGNNTANAATNNTGGCCGNGTTTAAANNNNNGANGGG
AAACCNGGNNTNCCANTAANNCTTGNNAAATCCCTTTTCC
BSK-1D9-1B - forward
CAGTTCCACCCGGGCAGGCAGTCGGGGGATGAGGGGCCGTCTAGCGTCCGCACCCGT
TCACTCCCAAGGAAGGTGTGTGGGCACGGTGAGGAGTGGGAAGAAACANAATANGAA
AGTGGCCTGACACGGGGATTCTAAGCANGTCANANNTATGNNGCTNG
BSK-1D9-1B - reverse
CTTTACTAAAAATACAAAAATTAGCCAGGCATGGTGGCAGGTGCCTGTAATCCCAGC
TATTCGGGAGGCTGAGGCAGGAGAATCACTTGAACCCAGGAACTGGAGGTTGCAGTG
AGCCGAGATCGTACCACTGCACTCCACCCAGGGTGACAGAGTGAGACTCCGTTGAAA
AAAAGAGAAAAAAAAATTAATACAAAGATATTAAAATTAAAAAGGAAAAATATCCCC
AGAACCCCATCACTTAAACAACAAATCAAATTTTTATTTTTCTCTTCCCATCCTACA
AGGCAACATAACTCTGACCTGCTTAGAATCCCCGTGTCAGGCCACTTTCCTATTCTG
TTTCTTCCCACTCCTCACCGTGCCCACACACCTTCTTGGGAGTGAACGCGTGCGGAC
GCTAGACGGNCCCTCATCCCCCGACTGCCTGCCCGGGTGGAACT
BSK-1K9-A4 - forward
CTGCGTCAGATTAAAACACTGAACTGACAATTAACAGCCCAATATCTACAATCAACC
AACAAGTCATTATTACCCTCACTGTCAACCCAACACAGGCATGCTCATAAGGAAAGG
TTAAAAAAAGTAAAAGGAACTCGGCAAATCTTACCCCGCCTGTTTACCAAAAACATC
ACCTCTAGCATCACCAGTATTAGAGGCACCGCCTGCCCAGTGACACATGTTTAACGG
CCGCGGTACCCTAACCGTGCAAAGGTAGCATAATC
BSK-1K9-A4 - reverse
GATTATGCTACCTTTGCACGGTTAGGGTACCGCGGCCGTTAAACATGTGTCACTGGG
CAGGCGGTGCCTCTAATACTGGTGATGCTAGAGGTGATGTTTTTGGTAAACAGGCGG
GGTAAGATTTGCCGAGTTCCTTTTACTTTTTTTAACCTTTCCTTATGAGCATGCCTG
TGTTGGGTTGACAGTGAGGGTAATAATGACTTGTTGGTTGATTGTAGATATTGGGCT
GTTAATTGTCAGTTCANTGTTTTAATCTGACGCA
BSK-1L3-B5 - forward
GGGGGNNGGGTTTTTTTTAAAAAAANANTGNACATTTATTTATTACTGNCCCTATTT
ATTAAANNGACTTTTTNTNAACCAAGGGCTTTTACTTTTTNTTCTTGCCTTTANGGG
CTTCAGGGGGTTTTCCCTTAANTACAACCAANTNTTTTTTTNAANCNAAAANTTTNN
CCACCTNCNNANCAACCTCNTTNTTGNCTGCCTTTTGTGCTTTNAANTNTCGGACAG
TTTGNAAGTCCTCAAANACCTNNAGGNNGAAATAANATTTNNCCCANCNANCCCATT
NNTGGGTATACANCNGAAGGAATATAAATNACTNTTTTANAAAAACACNNCCCATNT
TTNTTNCTNNNNNNTNTTTANAACANCCCCNANATNAAATNAACCNAATNNCCNTNN
NNGNGGATTNCNCCNNNCTNNCGGCTCAAAAA
BSK-1L3-B5 - reverse
CACTGATGGGCATTTGGGTTGATTTCATGTCGTGGCTGTTGTGAATAGTGCTGCAGT
GAACATACATGTGCATGTGTCTTTATGATAGAGTGATTTATAATCCTTCAGGTGTAT
ACCCAGTAATGGGATTGCTGGGTCAAATGTTATTTCTGCCTCTAGGTCTTTGAGGAC
TTGCAAACTGTCCGAGAACTGAAAGCACAAAAGGCAGACAAGAACGAGGTTGCTGCG
GAGGTGGCGAAACTCTTGGATCTAAAGAAACAGTTGGCTGTAGCTGAGGGGAAACCC
CCTGAAGCCCCTAAAGGCAAGAAGAAAAAGTAAAAGACCTTGGCTCATAGAAAGTCA
CTTTAATAGATAGGGACAGTAATAAATAAATGTACAATCTCTATATTAAAAAAA
BSK-1L3-C1 - forward
CCTTTCTATTAGCTCTTAGTAAGATTACACATGCAAGCATCCCCATTCCAGTGAGTT
CACCCTCTAAATCACCACGATCAAAAGGGACAAGCATCAAGCACGCAGCAATGCAGC
TCAAAACGCTTAGCCTAGCCACACCCCCACGGGAAACAGCAGTGATTAACCTTTACC
AATAAACGAAAGTTTAACTAAGCTATACTAACCCCAGGGTTGCTCAATTTCGTGCCA
GCCACCGCGGTCACACGATTAACCCAAGTCAATAGAAACCGGCGTAAAGAGTGTTTT
AGATCACCCCCTCCCCAATAAAGCTAAAACTCACCTGAGTTGTAAAAAACTCCANTT
CACACAAAATAGACTACNAAAGTGGCTTTAACATATCTTAACACACAATAGCTAANA
CCCAAACTGGGATTAGCGGAATCCCCG
BSK-1L3-C1 - reverse
CGGATTCCGCTAATCCCAGTTTGGGTCTTAGCTATTGTGTGTTCAGATATGTTAAAG
CCACTTTCGTAGTCTATTTTGTGTCAACTGGAGTTTTTTACAACTCAGGTGAGTTTT
AGCTTTATTGGGGAGGGGGTGATCTAAAACACTCTTTACGCCGGTTTCTATTGACTT
GGGTTAATCGTGTGACCGCGCTGGCTGGCACGAAATTGACCAACCCTGGGGTTAGTA
TAGCTTANTTAAACTTTCGTTTATTGCTAAAGGGGTAATCACTGCTGTTTCCCNTGG
GGGTGTGGCTANGCTAAACGTTTTGAGCTGCATTGCTGCGNGCTTGATGCTTGTCCC
TTTTTATCATGGNGATTTATAAGGGGAACTCCCTCNAATGGGGATGCTCCNTGTGTN
ATCTTACTANNANCTNTANAAAGGGGGGNTTNNNCTNANCGCCGGNNCTCCATAACA
ANAGGNNNGGNGGNNAAAAAATAATAANCGNCNGGNCATNTTAGCCNAATATTCNGA
NATC
BSK-1L3-A3 - forward
CAAAAAGATAATTAACCTTTATTATTCATTAAAAATGAGCTTTCTAAAATATTAGTA
AATTTCATTTTAAGCTCTGTCTTGAAGTGCTGATACCACTGAAGTAACATTTTTCTT
CTTTCAATTTTTTCTTGTAAAATTATAGTTTTCTCTTTTTCTAAAACAGCAGGGAGT
TCCTTCCAGTTCTTGATAAAGATAAAGGGAGCACCCATGCACTTGAGTAACTGCAGA
GGAGCACCCGTGGTGCACAGATGTATTCCCACACTTGCCACCTGTCATCACGTCTTC
CACCACAGGAATGGAGCCATANGAGCAAGCCTCATANATTCNATAGCATTCTCTGNT
TACTCCGACCAGGCACAATGTGAGATCACTCTGAANCAAGGCATCTTGGTAATTCTT
AAACTTTCATTTGTTTCTGAGGCTGCCANNGNTNTNTTNCTGAACCCACAAACTTAT
NNNTCCATCTTTTTTAAAANGTTCATTATGCCNGCTGGATNAANTTTNNAAATNNNT
CCTAANAANNACNTAATATCGCTT
BSK-1L3-A3 - reverse
GCCCCCGCGCGGGAGAGACGCGGCCGAGAACAGTCCACTTTGGAAAGTGAAGAATGG
AATCCTTGGGAAGGAGATGAAAAAAATGAGCAACAACACAGATTTAAAACTAGCCTT
CAAATATTAGATAAATCCACGAAAGGAAAAACAGATCTCAGTGTACAAATCTGGGGC
AAAGCTGCCATTGGCTTGTATCTCTGGGAGCATATTTTTGAAGGCTTACTTGATCCC
AGCGATGTGACTGCTCAATGGAGAGAAGGAAAGTCAATCGTAGGAAGAACACAGTAC
AGCTTCATCACTGGTCCAGCTGTAATACCAGGGTACTTCTCCGTTCATGTGAATAAT
GTGGTACTCATTTTAAATGGAAGAGAAAAAGCAAAGATCTTTTATGCCACCCANTGG
TTACTTTATGCCAAAATTTAATGCAAATTCAAAAACTNCACATCTTGCTTGTTGGTT
TGCTCGGAAATGAACATTGTGATAATGAGTGGATAAACCCATTCTTAAAAGAAATGG
AGGCTTCGTGGACCTGNTTTTCATAATATATACACCCCTGGATAATGACTGGATGTT
TTTANTGGCCTTAGGAGTAGCACATCCGGAATTTCTTGGGNGAAGCAANTGGCAATG
TTCTNATAAAGGCCTTTTATTAATTTTTAGAACNATTTTAAAATTNTCCNACGGCCT
ATAACTTTTAAAAAAAGGGACCAAACTTTTGGGTTACANAAAACTGCNCTNGGAAAA
ANAAGTTAAAATTCCAAA
1M13-A4 - forward
GAAAGTTTAGAAACTTTAAAACAATAATAATGACGGTGATAGTGATAATAATTGCTA
ATGCTTTCAGATCACATATGTGTTAGGCGCTGTTTTTTGTTGTTGTTGTTATTGTTG
AGGCAGTCTCACTCTGTTGGCCAGGCTGGAGTGCAGTGGTGCTTTGCCTCCTGGGTT
CAAGGGATTCTCCTGCCTCANCCTCCTGAGTAGCTGGGATTACAGGCATGCGCCACC
ACGTCGGGCTAATTTTTGCATTTTTAGTGGAGACGGGGTTTCATCATGTTGGCCAGG
CTGGTCTCGAACTCACGACGTCNAGTGATCCACCTGCCTCGGCCTCCCAAAGTGTTG
GGATTACAAGGCGGTGAGCCACCATGCCCACCCGCACTGNNTTAAATGCTTTACATA
TATTATCTCATTTAATCCTCNNAAACCTTACAATATANANACTACNATTATTTCCCT
TTATATTTATNGNNCTCTTAGGCTCANAAAAGGGAACTAACTTCTTGGTNCATGGNN
GGGGNNGGAATNAAANCCANGNNANCCGCTCCCNAAANTNCNTTCNNTGCCNNNCTN
ACTGGGCCNTCTTTNAAGCGGGGCC
1M13-A4 - reverse
TTCATGGGGAATAATTGCAATCCCCGATCCCCATCACGAATGGGGTTCAACGGGTAC
CCGCGCCTGCCGCCGTAGGGTAGGCACACGCTGAGCCAGTCAGTGTAGCGCGCGTGC
AGCCCCGGACATCTAAGGGCATCACAGACCTGTTATTGCTCAATCTCGGGTGGCTGA
ACGCCACTTGTCCCTCTAAGAAGTTGGGGGACGCCGACCGCTCGGGGGTCGCGTAAC
TAGTTAGCATGCCAGAGTCTCGTTCGTTATCGGAATTAACCAGACAAATCGCTCCAC
CAACTAAGAACGGCCATGCACCACCACCCACGGAATCGAGAAAGAGCTATCAATCTG
TCAATCCTGTCCGTGTCCGGCCCGGGTGAGGCAGTGAGCTGAGATTGCGCCACTGCA
CTCCAACCTGGGCGACAGAACGAGACCCCATCTCAAAAAAAAAGGGGGGGGTGGACA
GGGGGCAAGTGGAGTCTGGCTGCCAAAACTACTTGTTGATGGTGGGGAAAAAAAAAT
GGGTGTCTCCTCCCTTGTCACTGGGAAGGNTTTGGTTCTCTTTCATCTTCATCCTCT
ATAGGGCCTTTATTCTTCCCCAATCTGAAATATTTCGNGNGTTTTACTTNCCCACAT
CCTTCAAAAATATTTGAGGTTGATAAAANTAACTTCCGGGTTGGACCCNGGNTAATC
TTTGGGTNTTTGGNTTACCAAAAAAAGGGTCNCATTAACCCNTTTTGATT
BSK-2A11-A2 - reverse
CTCAGCCCTGAGAAAGGAGACATGTAACAAGAGTAACATGTGTGAAAGCAGCAAAGA
GGCACTGGCAGAAAACAACCTGAACCTTCCAAAGATGGCTGAAAAAAGATGGATGCT
TCCAATCTGGATTCAATGAGGAGACTTGCCTGGTGAAAATCATCACTGGTCTTTTGG
AGTTTGAGGTATACCTAGAGTACCTCCAGAACAGATTTGAGAGTAGTGAGGAACAAG
CCAGAGCTGTGCAGATGAAGTACAAAAGTCCTGATCCAGTTCCTGCAGAAAAAGGCA
AAGAATCTAGATGCAATAACCACCCCTGACCCAACCACAAATGCCAGCCTGCTGACG
AANCTNCAGGCCAGAACCAGTGGCTGCAGGACATGACAACTCATCTCATTCTGCGCA
CTTTAACGAGTTCCTGCANTCCACCTGAGGGCTTTCGGCAAATGTACATGGGCCCTC
AAATGGTGGTGGTAATGGCATTCTTNTTTGGCANAAACCTGTCCCTTGGCACAAACT
TATNTGGTNTTTTTGGAAACTAAAATNTAACGTTNGACCTATTTTAATATTTTAAAT
TATTAAANTTAAAATGNGAACT
BSK-2C5-A1 - forward
GCCAGAGGCTGTTCACCTTGGAGACCTGCTGCGGATATGGGTACGGCCCGGCGCGAG
ATTTACACCCTCTCCCCCGGATTTTCACGGGCCAGCGAGAGCTCACCGGACGCCGCC
GCAACCGCGACGCTTTCCAAGACACGGGCCCCTCTCTCGGGGCGAACCCATTCCAGG
GCGCCCTGCCCTTCACAAAGAAAAGAGAACTCTCCCCGGGGCTCCCGCCGGCTTCTC
CGGGATCGGTCGCGTTACCGCACTGGACGCCTCGCGGCGCCCATCTCCGCCACTCCG
GATTCGGGGATCTGAACCCGACTCCCTTTCGATCGGCCGAGGGCAACGGAGGCCATC
GCCCGTCCCTTCGGAACGGCGCTCGCCATCTCTNANGACCGACTGACCCATGTTCAA
CTGCTGTTCACATGGAACCCTTCTNCACTTCGCCTTCAAAGTTCTCGTTTGAATATT
TGCTACTACCACCAANATCTGACCTTGCGGNGGTTCNCCCGGCCCGCGCCTAGCTTT
AAGCTNANCGAACGGCCTTCTATCTNNNGGNGTACGTCCNGGGGTTCGGGGCGGGAG
CGCGGAATCANTNACCCCGCCGCNCCTTACAANTGCTNTGGGGNNAAANAAAATAAN
CGGCNGGCCTGNNAGCCAATTCA
BSK-2C5-A1- reverse
CGCTCCCCGCCCCCGGAGCCCCGCGGACGCTACGCCGCGACGAGTAGGAGGGCCGCT
GCGCTGAGCCTTGAACCCTAGGGCGCGGGCCCGCGTGCAGCCGCCGCAGGTGCAGAT
CTTGGTGGTAGTAGCAAATATTCAAACGAGAACTTTGAAGGCCGAAGTCGAGAAGGG
TTCCATGTGAACAGCAGTTGAACATGGGTCACTCGCTCCTGAGAGATGGGCGAGCGC
CGTTCCGAAGGGACGGGCGATGGCCTCCGTTGCCCTCGGCCCGATCGAAAGGGAGTC
GGGTTCAGATCCCCGAATCCCGAGTCGCGGANATGGGCGCCCGCGAGGCGTCCAGTG
CGGTAACGCGACCGATCCCGGANAACCCGGCGCGAGCCCCGGGGAGAAGTTCTCTTT
TCTTTGTGAAGGGCANGGCGCCCTGGAATGGGTTCGCCCGAGAGANGCGCCCGTGTC
TTGGAAGCGTNNNCGNTNCGGCGGGGTCCGGNGAGCTNTTNNTGGNCCCTGAAAATC
CGGGGAAANGGGGNAAATTTNNGCCGGGCCNACCCNTNTCCNANNAGGTTTCCAGGC
GAANANC
BSK-1E15-3 - forward
ANNAAAGTTGGANCCNTTNNTNCGACTACTATAGGGCGAATTGGGCCCTCTAGATGC
ATGCTCGAGCGGCCGCCAGTGTGATGGATATCTGCAGAATTCGCCCTTGGGATTGGT
GGCGACGACTCCTGGAGCCCGTCAGTATCGGCGGAATTCCGGCTTGTGCGTCAGGGA
GCACACCAGGCAAGAACCCCAGGTGGTGATGGCTCCAGAGGTTCTGAGAAGGAACAC
GCACAGGGCACACTCGGACGGCACAGGAAGTGAGGCTGCGGGTGGCCGGCTGGGCTG
CAGGGCTGCGGTGGGAAGCCCAGAACAGGGGCGCACCTTGCTCAGCAGGAAAAGCCC
ATGGGGAGGGGGTGAGCACGGAGCCAGGGCTCTCTGAAGTGTCCAGGTGCAGGGCAA
GGTGCCCACACACCATAAGGCACTTAAATGGCCACAAAGTCATCTCAGAAGAGTAAT
ATGACAAGTGCCTGGTCTCTAAAAAGCACAAGGGTGACCTCTGCATAGAAACAGTCC
CCCACCCCATCAGGCTGCCAGGGCAGGCTCACCTGGCGTCAAAACGATAGGATCAGG
CTCCCCTCGGTTCCCATAGTAGCAAATGACGTCTCCCTTTGCTGTGCTGCAGGCCAA
GGGGAGAGAATGTCAGACTACAGCCATAGGGCGTCCTCCCCGACACTGCCCGGTGAT
CTCACAGCCCTGTTTCTGGAGGCTAGCGATGTGCGCAGTAACCCGCTGCGGCCAGGT
CANGACTTGCAGGACCCANCCCTNANGTGCCTGCAGCCGGGTCATGGAGGCCCCAAC
TNTTGTCCACATTACNGGTATGTTCCCAACTNACTTNGGGGCCACCAGCCCAGAGGC
NCAGGATCTAAAAGGCCTCTTGCATCCCAATCCCATGGNATGGACCCACAAAGNTTG
CCTTGGGACANTTNAGGGCTGANCCCCTTNCCNCATTACCAACTTTTCATTAGGCCC
TTACCAAAGTTANTTNTAAGGTTTTTGGANAGNGGCCCNTTNGCCCANGNGATTAGG
TTCAAAAAAGGCCAAAACCATTTTNTTACTTNAANGTAAGGGCNNTTAAAAATTNGG
GNCTTAANTGGCTTNNGNNNTCCTTTTCCCTTNTNNGGAANANGGGGGGGNCTTTTT
GCCCNNTTTAAACC
BSK-1E15-3 - reverse
TGTGTGATGTGGTATTGAGGATTGGAATATTTTCAGCTGGGGAGCATTTTGAACTAT
TTTCTTTTTAAAAAATGATTTATTTATTTTGTAGAGATGGGGTCTTGCTATGTTGCC
CAGTCTGGTTTCAAACTCCTGGCCTCAAGCAGTCCTCCCTCCTTAGTCTCCAGATTA
GCTGGGACTATAGGCATGAGTCACTATGCCCAGCTCTACTGTCCCTTAACTATTTTA
AAGTGTACACTTAACACTTGAGAGTAGGAAAATGTGGTCTTTGGCGCTTTCTTTGGA
AGCCTCGATCACCCTGGGGCCACACTGGGGCCACCTGTCCAGAAACCCTCATAGATG
AGCTGTGGTAAGGGGCCTGATGGAGAGGTTGGTGATGTGGGGATGGGGCTCAGCCCT
GCAGTTGTCCCCATGGGCAGCCTCTGTGTGTCCATGCCATGGGGCATTCGGCATGGC
AGGAGGGCATTTTAGATCCTGTGCCTCCTGGGCTGGTGGGCCCCGAGGTGAACTGGG
AACATACCCGTGATGCTGGACAAGANCTGGGGCCTNCATGACCCGCTGCCAGCACCT
GANGGGCTGGGTCCTGCAAGCTCCTGACTGGCCGAACCGGCTACTGNGCACATCGGT
AGCCTCCANAACAAGGCTGTGANNCACCCGGCATGTCNGGGAGGACNCCCTNTGGTG
GAANCTNACATTNTTTCCCTGGCCTGAAGNCANCAAAGGGAGACGCATTTGCTCTAT
GGGAACCNNGGGGGACCTGNNCCTATNNTTTACCCNGGNGAACCTCCTTGNAACCNN
NNGGGTGGGGNCGTTTTNTCCAAGNNACCTTGCTTTTTAAAACCNGNCTTTGNAAAN
ACNTTT
BSK-1H5-4 - forward
CGCGGGATTCCGGTGAGAAGTATCCGCGACGAGCTATCCGGGAAAGGGCCGAATGCG
ATCAAACCTAATCCGCGAGACTTGCTAAAATTCTCCAAGTCCCGGCTGCTTATGTAC
CTATCGAGCCAGATTCACCACCGTCATTATCAACTCATTTTATGAAAAAATGTAATC
TAAAGTATATCCTTGTTGAAAAAAAACAAATTAATGTATTCTTTATGACCAGTAGGA
GTTGGACATAGCAAAACCCAAAAAGGAGTTGGGCGCAGCAAAACCTTGCTTCCTATC
CCATGATTTTGATGATGGTGTAAGTGTTCTTCCTTCATTTAACACAGGAACGATCAA
AATTTAAATCTTTTCATGAAACATTATTGAACTATGATACATTTACAGTGGAACATA
ATGACCTAGTGCTCTTCAGACTTCACTGGTAAAATACTGAGGT
BSK-1H5-4 - reverse
ATTATTTTTGACACCAGACCAACTGGTAATGGTAGCGACCGGCGCTCAGCTGGTTCA
TATTTCTCTTTTCCATCATTTAGCATCAAGTTCACCTCAGTATTTTACCAGTGAAGT
CTGAAGAGCACTAGGTCATTATGTTCCACTGTAAATGTATCATAGTTCAATAATGTT
TCATGAAAAGATTTAAATTTTGATCGTTCCTGTGTTAAATGAAGGAAGAACACTTAC
ACCATCATCAAAATCATGGGATAGGAAGCAAGGTTTTGCTGCGCCCAACTCCTTTTT
GGGTTTTGCTATGTCCAACTCCTACTGGTCATAAAGAATACATTAATTTGTTTTTTT
TCAACAAGGATATACTTTAGATTACATTTTTTCATAAAATGAGTTGATAATGACGGT
GGTGAATCTGGCTCGATAGGTACATAAGCAGCCGCGACTTGGACAATTTTAGCAAGT
CTCGCGGATTAGGTTTGATCGCATTCGCCCCTTTCCCGGATAGCTCGTCGCGGATAC
TTCTCACCGGAATCCC
BSK-36-8 - reverse
CGGAATCCGGTGGACGCCGTGCCGTTACTCGTAGTCAGGCGGCGGCGCAGGCGGCGG
CGGCGCCATAGCGCACAGCGCCCCTTAGCAGCAGCACCAGCAGCAGCAGCATCGGAG
GTACCCCCGCCGTCGCAGCCCCCGCGCTGGTGCAGCCACCCTCGCTCCCTCTGCTCT
TCCTCCCTTCGCTCGCACCATGGCTGATCAGCTGACCGAAGAACAGATTGCTGAATT
CAACGAAGCCTTCTCCCTATTTGATAAAGATGGCGATGGCACCATCACAACAAAGGA
ACTTGGAACTGTCATGAGGTCACTGGGTCAGAACCCAACAGAAGCTGAATTGCAGGA
TATGATCAATGAAGTGGATGCTGATGGTAATGGCACCATTGACTTCCCCGAATTTTT
GACTATGATGGCTAGAAAAATGAAAGATACAGATAGTGAAGAAGAAATCCGTGAGGC
ATTCCGAGTCTTTGACAAGGATGGCAATGGGTATATCAGTGCAGCAGAACTACGTCA
CGTCATGACAACTTAGGACAAAAACTAACAGATGAAGAAGTAGATGAAATGATCAGA
GAAGCAGATATTGATGGAGACGGACAAGTCAACTATGAAGAATTCGTACAGATGATG
ACTGGCAAATGAACACTACTTTAACTCCTTTTCCCCTNTAGAAGAATCAAATTGAAT
CTTTACTTACCTCTTGC
BSK-83-1
GTTCAAACAGCAAACGCCCACACATGGCCCAGAGGTGGTGGTAGTCAGGGTGTGTGG
GTGTTTTTAGGGTTCTTTAGTGTTGTTTCTTTCACCCACGGGTGGTGGTCCCAGCCA
GTTTGGTGCTGACGGTGAGAGGAAATTAGAATCTGTTTGCAAATTGTCCAACCCACC
CCCTCAACATGAGGGGCTTCCATTTTCTGTGTTTTGTAAGGGAACTGTTTCCTTCAT
GCCGCCATGTTCCTGATATTAGTTCTGATTTCTTTTTAACAAATGTTATCATGATTA
AGAAAATTTCCAGCACTTTAATGGCCAATTAACTGAGAATGTAAGAAAATTGATGCT
GTACAACGCAAATAAAGCTCTTTATTAACCTTG
BSK-2G12-A5 - forward
GCACACTGGCGGCCGTTACTAGTGGATCCGAGCTCGGTACCAAGCTTGATGCATAGC
TTGAGTATTCTATAGTGTCACCTAAATAGCTTGGCGTAATCATGGTCATAGCTGTTT
CCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCTGCATTAATGAATCGGC
CAACGCGCGGGGAGACGCGGTTTGCGTATTGGGCGCTCTTCCGCTTCCTCGCTCACT
GACTCGCTGCGCTCGGTCGTTCGGCTGCGGCGAGCGGTATCAAGCTCACTCAAAGGC
GGTAATACGGTTATCCACAGAATCAGGGGATAACGCAGGAAAGAACATGTGAGCAAA
AGGCCAGCAAAAGGCCAGGAACCGTAAAAAGGCCGCGTTGCTGGCGTTTTTCATANG
CTCCGCCCCCTGACAGCATTACAAAAATCGACGCTTCAAGTCAGANGTCGCGAACCC
GACAGGACTATAAAGATCCANGCGTTTCCCCTGGAACTTCCTCGGCGCTNTCTGTTC
GACCCTGNCGTTACCGGAACCTGTCCGCNTTNTCCTTCGGAAGCGNGGGCTTTNTAT
ACTTACGCTGAAGTATCTNATTCGGGGAGNCGTCGNTCAACTGGCTGGGNGCACAAC
CCCCGTTAGCCGACGTGNGCTTACCGGAATNTNGNTGGTCAACCGCNANACCANTAT
CGCNTGNNNANCNTGNACAGATACCANCAGGTTTAGGGGGTTCAAATTTAAGGGGCC
ATCCGTANTAAAAACAATGGTTTCCNG
BSK-2G12-A5 - reverse
CAGATATCCATCACACTGGCGGCCGCTCGAGCATGCATCTAGAGGGCCCAATTCGCC
CTATAGTGAGTCGTATTACAATTCACTGGCCGTCGTTTTACAACGTCGTGACTGGGA
AAACCCTGGCGTTACCCAACTTAATCGCCTTGCAGCACATCCCCCTTTCGCCAGCTG
GCGTAATAGCGAAGAGGCCCGCACCGATCGCCCTTCCCAACAGTTGCGCAGCCTGAA
TGGCGAATGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTC
CCTTCCTTTCTCGCCACGTTCGCCGGCTTTCCCCGTCAAGCTCTAAATCGGGGGCTC
CCTTTAGGGTTCCGATTTAATGCTTTACGGCACCTCGACCCCAAAAAACTTGATTAA
CGGTGATGGTTACGTAGTGGGCCATCGCCCTGATAGACGGGTTTTCGCCCTTTGACG
TTGGAGTCCACGTTCTTTAATAGTGGACTCTTGGTNCAACTGGGACAACACTTAANC
CTATCTCCGCTATTCTTTTGATTATAAGGGATTTGGCCATTCGCGCTATTGGTTAAA
AAACACTGATTAACAAAATTTAACGCGAATTTACAAATTCAGGCCCAAGGCTGTAAG
GAANCGACACTAAAAGCCATCCGAAAACCGGTTANCCCGATAAAGGAACTATGGGTT
TTGGGAAAGGAAACCAACCCAAAAAAGCGNACTTNAAGGGCTACTGNAAAGTAAANG
GNGTTATGAAGAACAACGATGCANNGCCCCTTGAAGTGGAACCCGAAAAATGAGGTT
TTG
BSK-2G14-A2 - forward
GTGGTTTTGCTTTGTTCTTACTAGGTTTTCGTGCCACCTTCCCTGCCTGCGCTTGTG
CCCCCTCTCCTCCTTCGCACTGGCGGCCTCCTTGCCTCCCTTCCACCCGTGCTGCCA
TCCCGTGCCTGTCGTGTTGGTTCTTCACACGTGCTCTGTTCTCGGGGTTGTTCCATT
CATGCCTTCTTCGAGGGTGAGGGTGGCTTGGGAACCGACCCAGTGATCATGCCTACT
TTCTTCTTTCTATCTCCCTCCTTCCCAGCCCACCCGGGCAGCAGACTCTGATGGAAG
GAAGGTGCCCTAGGTGGGCTTTTAGAAACTAACCGGACTGGTTTTCAAAGCAGTTAT
CTTGGGAAACTGTTTATTCCAGCGATGTGACTTTTTTCAGAATATTTCTTGGAATCA
TATTCANAGTCTGGGGCTGTGTGTTCAGCAGCCTTAAGGATGCTAGACACTCATTTA
GTGCCCAAGGAGTCCAGCGAATGACGTCTGNGGCAACCAGGCTCAGNGCAAGCAAAA
GGACCATTTAAAGTAAATACTTGGATCAATCTGTGACTCTTAAATGGCTNAAAAGAA
TTTGNATTCAAAAGGGTTGAACCCTGGCACGTTGGCNTGGGAGCTATANCTTGATCC
TTGGANAAAAATTCATTGGTGGGGAACTGATTGGTNGGANAAANTGGCTGCTACTTN
TGGNATCCAGGTNTGACTTACAGGGAAAAAAAAAAAACGCGANTGANGGTCAGATNN
NNCCCATCANCCATTCACCATNGGGCNNNTANGGTCCNCCNGNCAACTNAGCAANTG
NATTNATNGGCCCAAAACTGGGAACNGGCNATTTCNNG
BSK-2G14-A2 - reverse
GCCCCTGGTAAAAGTCAGAACCTGGGATGACCAGAAAGTAACAGGACAGATTTCTCC
CAGCAAATCAGTCTCCACAACCAAATGAATATTGTTCTCCAAGGAGTCAAGCTATAG
ACTCACAATGACAACGTGGCCATGGCTCAAAACACTCTCTGAAATTACAAAATTGCT
TTCTGAGCCAATTTAAGAGTCACATGATTGAATCCAAGCTATTTTACTTTAAATGGT
CCTTTTGCTTTGCACCTGACACCTCGCTTGGCCACAGACGTCATTCGCTGGACTCCC
TGGGCACTAAATGAGTGTCTAGCATCCTTAAGGCTGCTCAACACACAGCCCCAGACT
CTGAATATGATTCCAAGAAATATTCTGAAAAAAGTCACATCGCTGGAATAAACAGTT
TCCCAAGATAACTGCTTTGAAAACCAGTCCCGTTAGTTTCTAAAAGCCCACCTACGG
CACCTTCCTTCCATCANAGTCTGCTGCCCGGGTGGGCTGGGAAGGAGGGAGATACAA
AGAAGAAAGTAGGCATGATCACTGGGTCGGTTCCCAAGCCCCCTCACCCTTCAAGAA
GGNATGAATGGACAACCCCGAGAACAGAGCCGTGTGAAGACCACCNACNGCNCGGAT
GCCACACGGTGGAAGGAGGCAGGAGGCCNCNGTGCCANGANGANAGGGCNCAACCCA
GCCGGAAGNGGCCCAAACCTATACAACAAGCAAACCCCGGATTCNCTGACCCGGCNT
ACCTACCATNGGNGGGNNAAANATATACCGGCGGCTGCAGCCAATTGAAATCATAAC
TGNGGCGTCACTGCTTNAGGCCATTNCCTANGGGGATAAATNTGCGGGTTNACGGCC
G
BSK-2H11-B3 - forward
AGAAATCTTTTAATGTTTATTCAAAGGACAAAATAAAGACTATGAACCAATGAGACA
CATAGTAAAAAAGTACAATTTTAATATAGTGAATGTAATATATATGTAATTACTCAT
AACAAAATGGTCAAAACCTTTAAAAGATACACAATAGGCATCTAAAAAGCTCAGCAA
TGCTAAATATATAATATATATTATATATAAATATATAATATATAAATACATACGTTT
TTACCAAGAAATGTTTTATTTTTCTTCCAGTAGCTTTGTTAATTGCACAAAATTATG
TTTTGTTTTTGCCATTTAAATATTATCACAGAATCCTATTCTGAAAGACAAATGTTC
ATTAAAAACAAAGCAAAAATAGAAATTCACAACCATTAATTACCTAGGTTTGTCATT
TAAAGCTTTAAAGAAAAAAAGGGAGGAGCTTTCCTACAAGCCTTTTCCAAGTGTCAC
ATTTTCTCTTTAAAAGGGAAGGATTTNCAAACAAAGGTGAAATAGCTTAAACAGAAA
TATTTGTAAAAATAAACTTTANGCATTATCAAGGATATTAAGACACACTGACTAACC
CCGTTCATTACCCGNATCTTCCCCNCCCCACCCCAGTGGGTCCACCAGGACTAGAAC
AGNTTTACNTTANACAGAAATGCTTCAAATCCCAGGGAAAGAACTGGCTAAAANCCG
CAGGNTTTTNCTGCCTCCCGTGCCGTNGTTTTGAATCTTTACCAGGTTTCTTGGAAG
GGCCAACTGGAGTGGGAGGACTGCCACGGGCCCTTTTTATATGGATCNTGGGCCGCG
TCCTTCAGTGGTGGGGAAAAAAAACGGGGC
BSK-2H11-B3 - reverse
ATAATTATATATAAGGTGGCCACGCTGGGGCAAGTTCCCTCCCCACTCACAGCTTTG
GCCCCTTTCACAGAGTAGAACCTGGGTTAGAGCATTGCAGAAGACGACCGGCAGCGG
GGAGGGCAGGGAAGATGCCTGTCGGGTTTTTAGCACAGTTCATTTCACTGGGATTTT
GAAGCATTTCTGTCTGAATGTAAAGCCTGTTCTAGTCCTGGTGGGACACACTGGGGT
TGGGGGTGGGGGAAGATGCGGNAATGAAACCGGNTAGNNAGNGNTGNCTTAATATNC
TTGATAATGCTGNANAGNTTATTNTTACAAATATTTNTGTNTAAGCTATTTCACCTT
TNNTTGGAAATCCTTCCCTTTTAAAGANAANATGNGACACTTTGTGAANAGGCTTGT
NNGAAAGNTCNTCCC
BSK-2H11-A5 - forward
AAGAAATATGGGACTATGTGAAAAGACCAAATCTACGTCTGATTGGTGTACCTGAAA
GTGATGTGGAGAATGGAACCAAGTTGGAAAACACTCTGCAGGATATTATCCAGGAGA
ACTTCCCCAATCTAGCAAGGCAGGCCAACGTTCAGATTCAGGAAATACAGAGAACGC
CACAAAGATACTCCTCGAGAAGAGCAATTCCAAGACACATAATTGTCAGATTCACCA
AAGTTGAAATGAAGGAAAAAATGTTAAGGGCAGCCAGAGAGAAAGGTCAGGTTACCC
TCAAAGGAAAGCCCATCAGACTAACAGCGGATCTCTCGGCAGAAACCCTACAAGCCA
GAAGAGAGTGGGGGCCAATATTCAACATTCTTAAAGAAAAGAATTTTCAACCCAGAA
TTTCATATCCAGCCAAACTAAGCTTCATAAGTGAAGGAGAAATAAAATACTTTATAG
ACAAGCAAATGCTGAGAGATTTTGTCAACACCAGGCCTGCCCTAAAAGAGCTNCTGA
AGGAAGCGCTAAACATGGAAAGGAACACCGGTACCANCGNTGCAAAATCATGCCAAA
TGTAAAGACCTCGAGACTAGGAAGAACTGCTCACTAACGAGCAAATCCCACCTTACA
TCTTATGACGGGTCAATTCCCCNTACATATACTTTAATNTAATGGCTAANTCTGCAN
TAAAAGACNNGACTGNAGTTGGTAAGAGCAGACCTNATGNGTTGNTCNGAACCATTA
CTGNNAACCCNNGGTCAATAAGGTGNAAGATTNCNGCCTGGAACAAAAGNGGGTGGA
TCTACTTGTAACCGCTTTACCNCAAACAAAAACAAAGGCTTCTTTGNANGGTCATCC
CAAGNNTCNTN
BSK-2H11-A5 - reverse
GTTCTGTAGATGTCTATTAGGTCCGCTTGGTGCAGAGCTGAGTTCAATTCCTGGGTA
TCCTTGTTGACTTTCTGTCTCGTTGATCTGTCTAATGTTCACAGTGGGGTGTTAAAG
TCTCCCATTATTAATGTGTGGGAGTCTAAGTCTCTTTGTAGGTCACTCAGGACTTGC
TTTATGAATCTCGGTGCTCCTGTATTGGGTGCATAAATATTTAGGATAAGTTAGCTC
CTCTTGTTGAATTGATCCCTTTACCATTATGTAATGGCCTTCTTTGTCTCTTTTGAT
CTTTGTTGGTTTAAAGTCTGTTTTATCAGAGACTAGGATTGCAACCCCTGCCTTTTT
TTGTTTTCCATTGGCTTGGTAGATCTTTCTCCATCCTTTTATTTTGAGCCTATGTGT
GTCTCTGCACGTGAGATGGGTTTCCTGAATACAGCACACTGATGGGTCTTGACTCTT
TATCCAACTTGCCAGTCTGNGTCTTTTAATTGCAGAATTTAGTCCATTTATATTTAA
AGGTAATANTGGTATGNGTGAATTGATCTGNCATTATGATGTAGCTGGNGATTTGCT
CGTAGTTGATGCAGTTCTTCTAGCTCATGGCTTACATTTGGCATGATTTGCACGGTG
GACCGGTGGTCCTTTCATGTTAACCTTCTTCAGAGCNTTTAGGCAGGCTGGNGTGAC
AAAACTTAACATTTGCTGGCATAAGATTATTCTCTTACTTATAACTTATTGGTGGAA
TNAATCTGGTGAAATNTTTTTAAAANTGAAATGGCCCCNTTTTNGGTTGAGGTTTTC
CAAANCNTTAACNNNGNTTCTTAGGACCCCG
BSK-2H12-A4 - forward
CTTTAAAGTAGTTTTTTCCAATTCAGTGAAGAAAGTCATTGGTAGCTTGATGGGGAT
GGCATTGAATCTATAAATTACCTTGGGCAGTATGGCCATTTTCATGATATTGATTCT
TCCTACCCATGAGCATGGAATGTTCTTCCATTTGTTTGTATCCTCTTTTATTTCCTT
GAGCAGTGGTTTGTAGTTCTCCTTGAAGAGGTCCTTCACATCCCTTGTAAGTTGGAT
TCCTAGGTATTTTATTCTCTTTGAAGCAATTGTGAATGGGAGTTCACTCATGATTTG
GCTCTCTGTTTGTCTGTCGTTGGTGTATAAGAATGCTTGTGATTTTTGTACATTGAT
TTTGTATCCTGAGACTTTGCTGAAGTTGCTTATCAGCTTATGGAGATTTTGGGCTGA
GACAATGGGGTTTTCTAGATATACAATCATGTCGTCTGCAAACAGGGACAATTTGAC
TTCCTCTTTTCCTAATTGAATACCCTTTATTTCTTCTCCTGCCTAATTGCCCTGGCC
AGAACTTTCAACACTATGTTGAATANGANTGGTGANAAAAGACATNCCTGCTTGGGC
CAGTTTCAAAGGAATGCTTCCAGTTTTGNCATTCATATGATATGGCTGGGGGTTGGC
ACAAAACTCTTATATTTGAAAACCGTCCACATACCAATTATGAAAGTTTAACTGAAG
GTGGTGAATTTGCAAAGCTTTTTGCACAATGAAAAACATGGGTTTGCTTGCCTNTAA
TCCGATACATATGATGGAATTGACNACTGCTCCAGGATANCCNTGACTGGGGNAACN
TTAAGGNGTGATCGTGCNNTTTTTGNGATTGCNAAGCCCAGG
BSK-2H12-A4 - reverse
GAGAAAATCTAGAAGAAATGGATAAATTCCTCGACACATACACTCTCCCAACACTAA
ACCAGGAAGAAGTTGAATCTCTGAATAGACCAATAACAGGATCTGAAATTGTGGCAA
CAATCAATAGCTTACTAACCAAAAAGAGTCCAGGACCAGATGGATTCACAGCCGAAT
TCTACCAGAGGTATAAGGAGGAGCTGGTACCACTCCTTCTGAAACTATTCCAATCAA
TAGAAAAAGAGAGAATCCTTCCTAACTCATTTTATGGGGCCAGCATCATTCTGATAA
CAAAGCCGGGCAGAGACACAACCAAAAAAGAGAATTTTAGACCAATATCCTTGATGA
ACATTGATGCAAAAATCCTCAATAAAATACTGGCAAACCGAATCCAGCAGCACATCA
AAAAGCTTATCCACCATGATCAAGTGGGCTTCATCCCTGGGATGCAAGACTGGTCAA
TATATGCAAATCAATAAATGTAATCCAGCATATAAACAGAGCCCAAGACAAAAACCA
CATGATTATCTCAATAGATGCAGAAAAAGCCTTTGACAAAATTCAACACCCTTCATG
CTAAAAACTCTCAATAATTANGTATGATGGACGTATTTCAAATAATAAGAGCTATTG
NGACAACCCCAGCCATTCTACTGATGGCAAACTGGGAGCATTCCTTGAAACTGGACA
GACNGGTGCTTNTACACTCTATCACTAGGGTGAAGTTGGCAGGCATAGCGGNANGAT
ANGGNTCATNGGAAAAGGAGCAATNCTGTTGNACAATGTGTTTAAAACCCTGGTACC
AATTCTACGTACATNGAACTNGTCAATANNCAATCAGNTT
BSK-2H9-A3 - forward
TGTAATCCCAGCACGTTGGAAGGTTGAGGCGGGTAGATCATGAGGTCAGGAATTCAA
GATCAGCCTGGCCGGGATGGTGAAACCCCATCTCTACTAAAAATACAAAAATTAGCC
AGGTGTAGTGGTGGGCGCCTGTGGTCCCAGCTACTATGGTGGCTGAGGTGCGAGAGT
CGCTTGAACCTGGGAGATGGAGGTTGCAGTGAGCCAAGATCGTACCACTGCACTCCA
GCCTGGGCAACAGAACAAGACTCCATTTCAAAAAAAGAAAATTCTTATTTGCCATGA
GCCGAGGAATGCACAGGTACTAACTAGATGGTGTGGACAGCTGACGCAAACTGGGCA
TATACAATGGGACACACCTGTACTAGGATGAAAGGCACAGCCTANAGGGCTGGCAGG
TGTTGGGTAATGCTCAAGTTTCAGAGTGATGGCAGAAGAGTAGGTTGGTAGGCCCTC
ATGGCTCTGCTTGGCAGCACNGAGTTCCGCGGAATTCCGCCATCTGACGGCTCCANG
AGTCGTCGCCCAATCCAAGCCGAATTNCACACACTGGCGGCCGTACTAGTGGATCCG
ACTCGGACCAACTTGATGCATAACTTGAGTATTCTATATGNCACCTAAATAGCTTGG
CGTAATCATGGCATACTTGTTTCTGNGNGAAATTGTATCCGNTACAATTCNCACACA
TACANCCGAAGCATAAGTGNAAGCNGGGGNCCCTAATGAGTGACTACTACTTATTGG
GTGGCTACTGCCGTTTCANCGGAAACTGCTGCNANTCTTATNATCGCCACCNCGGGA
ACNGGTGNGNTGGCNTTTCCTCTGTATTATCTGCTGCTTGGTGGGAACGGTA
BSK-2H9-A3 - reverse
CGGAACTCCGTCCTGCCAAGCAGAGCCATGAGGGCCTACCAACCTACTCTTCTGCCA
TCACTCTGAAACTTGAGCATTACCCAACACCTGCCAGCCCTCTAGGCTGTGCCTTTC
ATCCTAGTACAGGTGTGTCCCATTGTATATGCCCAGTTTGCGTCAGCTGTCCACACC
ATCTAGTTAGTACCTGTGCATTCCTCGGCTCATGGCAAATAAGAATTTTCTTTTTTT
GAAATGGAGTCTTGTTCTGTTGCCCAGGCTGGAGTGCAGTGGTACGATCTTGGCTCA
CTGCAACCTCCATCTCCCAGGTTCAAGCGACTCTCGCACCTCAGCCACCATAGTAGC
TGGGACCACAGGCGCCCACCACTACACCTGGCTAATTTTTGTATTTTTAGTAGAGAT
GGGGTTTCACCATCCCGGCCAGGCTGATCTTGAATTCCTGACCTCATGATCTACCCG
CTCACCTTCCAACGTGCTGCGATTACA
BSK-2I5-4B - forward
CTGTTTAATTAAAACAAAGCATCGCCAAGGCCCGCGGCGGGTGTTCACGCCATGTGA
TTTCTGCCCAGTGCTCTGAATGTCAAAGTGAAGAAATTCAATGAAGCGCGGGTAAAC
GGCGGGACTAACTATGACTCTCTTAAGGTAGCCAAATCCCTCGTCATCTAATTAGTG
ACGCGCATGAATGGATGAACGAGATTCCCACTGTCCCTACCTACTATCCAGCGAAAC
CACAGCCAAGGGAACGGGCTTGGCGGAATCAGCGGGGAAAGAAGACCCTGTTGAGCT
TGACTCTAGTCTGGCACGGTGAAGAGACATGAGAGCTGTAGAATAAGTGGGAGCCCC
CCGGCGCCCCCCCGGTGTCCCCGCGAGGGGCCCGGGCGGGGTCCGCCGGCCCTGCAG
CCGCCGGTGAAATACCACTACTCTGATCGTTTTTTCACTGACCCGGTGAG
BSK-2I5-B4 - reverse
CTCACCGGGTCAGTGAAAAAACGATCAGAGTAGTGGTATTTCACCGGCGGCCTGCAG
GGCCGGCGCACCCCGCCCCGGGCCCCTCGCGGGGACACCGGGGGCGCGCCGGGGGCC
TCCCACTTATTCTACACCTCTCATGTCTCTTCACCGTGCCAGACTAGAGTCAAGCTC
AACAGGGTCTTCTTTCCCCGCTGATTCCGCCAAGCCCGTTCCCTTGGCTGTGGTTTC
GCTGGATAGTAGGTAGCGACAGTGGGAATCTCGTTCATCCATTCATGCGCGTCACTA
ATTAGATGACGAGGCATTTGGCTACCTTAAGAGAGTCATAGTTACTCCCGCCGTTTA
CCCCCGCTTCATTGAATTTCTTCACTTTGACATTCAGAGCACTGGGCAGAAATCACA
TCGCGTCAACACCCGCCGCGGGCCTTCGCGATGCTTTGTTTTAATTAAACA
BSK-2I5-A5 - forward
CCCATTTAACTTTTTTAATGGGTCTCAAAATTCTGTGACAAATTTTTGGTCAAGTTG
TTTCCATTAAAAAGTACTGATTTTAAAAACTAATAACTTAAAACTGCCACACNCAAA
AAAGAAAACCAAAGTGGTCCACAAAACATTCTCCTTTCCTTCTGAAGGGTTTACNAT
GCATTGGTATCATTAACCAGTCTTTTACTACTAAACTTAAATGGCCAATTGAAACAA
ACAGTTCTGAGACCGTTCTTCCACCACTGATTAAGANTGGGGTGGCAGGTATTAGGG
ATAATATTCATTTANCCTTCTGAGCTTTCTGGGCAGACTTGGTGACCTTCCCAGCTC
CAGCAGCCTTCTTGTCCACTGCTTTGATGACACCCACCGCAACTGTCTGTCTCATAT
CACGAACAGCAAAGCGACCCAAACGTGGATAGTCTGAAAAGCTCTCAACACACATGG
GCTTGCCAGGAACCATATCAACAATGGCAGCATCCCAGACTTCAAGAATTTANGGCC
ATNT
BSK-2I5-A5 - reverse
AGATGGCCCTAAATTCTTGAAGTCTGGTGATGCTGCCATTGTTGATATGGTTCCTGG
CAAGCCCATGTGTGTTGAGAGCTTCTCAGACTATCCACCTTTGGGTCGCTTTGCTGT
TCGTGATATGAGACAGACAGTTGCGGTGGGTGTCATCAAAGCAGTGGACAAGAAGGC
TGCTGGAGCTGGCAAGGTCACCAAGTCTGCCCAGAAAGCTCAGAAGGCTAAATGAAT
ATTATCCCTAATACCTGCCACCCCACTCTTAATCAGTGGTGGAAGAACGGTCTCAGA
ACTGTTTGTTTCAATTGGCCATTTAAGTTTAAGTAGTAAAAGACTGGTTAATGATAA
CAATGCATCGTAAAACCTTCAGAAGGAAAGGAGAATGTTTTGTGGACCACTTTGGTT
TTCTTTTTTGCGTGTGGCAGTTTTAAGTTATTAGTTTTTAAAATCAGTACTTTTTAA
TGGAAACAACTTGACCAAAAATTTGTCACAGAATTTTGAGACCCATTAAAAAAGNTA
AATGGG
BSK-2K2-A1 - forward
CTGGGCTCTGGGCTAGTACTGGGGAGTATCTGCAGAATCCCGTGATATGATCCGTCT
TCAGCTAAAGATATTATTTCACAAGTGGAATGACAGCTGACTTCTCAACAACAACGA
AAGCAAGGAGACAGTTGAAAGACATCTTGAAAATGGAATTAGCAGTTCACAAAGCAC
ATTCGCATATAAGGGCTTGTTTTGAATTGATCTTGGCAGCAATTCTATGAAACAAGT
AAAAGCACAAGAGGAATAGGAACTGCACCTCTTCCTTCAGTTTCAGCTTGAATAATA
TCAGGAAGATTCGTATCGGTCTGAGTTGGGTCACGTACCCGACGTGCTATAGCTGAG
GATGGGGTAAGCTGATTCGAGTTTGCAACACTGTTCACAGAGCCAAGATATGGAAAG
AACCTAAATGTCAACTGGTGGATGAATGGATAAAGAAATTGTGGTATATACATACAC
TGGAATATTATTCAACCTTAAAAAGAAGGAAATCCTAACATTTGTGACAACATGGAT
GGACCTGGAGGGAATTATGCTGAGTGAAATAAGACAGACNCAAAAGACNTTTCTTGC
AGGAGCTCCTTATATGTGGAATCTAAATAGTCAGCTTAAAGAAGANAGTAAACTACT
GGTGTCAGGAGCAGGANAAAATGGAAATGAANAGGNGATAGTAAAGGGACAAAGTTC
AGTATCAANATAATAAGTTCTGGNGGTTACTATTAATANTCCATAGACCTATAATAC
CATACTGGTTGGTACTAAAATGCTAAAGGGTTTCTAATGTCTACCANANAAAANANA
NGGAAAATAAGGGCGGAGGCCCTNAAAGGGAGGATGTATGCCTGNGGGGAAGGTCTG
AAATCTNCCCACTATGNG
BSK-2K2-A1 - reverse
CACATACAAAAAGCTGTACATATTTAATATTTACATCTCAATTAGTTTGGGGATAAG
TATACTCTCATGAAACCATCACCACCATCAAGGCCATAAACATATCCATCACCTTTT
GAAGTGTCCTCCTGCCCCTTAATTATTACCATTATTATTATTATTATTGGTAAGAAC
ATATAAGATATACCCTCTTAGCAATTTTAAGTATACAATACAGTATTGNTACTTATA
GGTACTATGTGATATATTAATAGTAAACCTCCAGAACTTATTTATCTTGTATAACTG
AAACTTTCTACCCTTTAACTATCACCTCTTCATTTCCACTTTTCTCCTGCTCCTGAC
AACCAGTAGTCTACTCTCTTCTTTAAGCTTGACTATTTTAGATTCCACATATAAGTG
AGCTCCTGCAAGAAAGNCTTTTTGNGTCTGCTTATTTCACTCAGCATAATTCCCTCC
ANGTCCATCCATGTTGTCCAAATCGTAGGATTTCCTTCTTTTTAAGGTGAATAATAT
TCCAGTGTATGNATATACCACAATTTCTTATNCATTCTTCACCAGTGACATTAAGGT
CTTTCTATCTTGGCTNTCGGAACAGGGTGCAAACTCCAATCAACTTACCCATCCTAA
CTATAGACGTNGGTACGGACCCACTAAACGAACGAANTTCTGNNTATTAACTGAACT
GANGANAGGGGAGTCCATNCTTGGCTTTACTGGTCAAAAATGNGGCANACAATAAAA
ACCTTTTGCAACGGTTGGGACGTATCATTNANAGNTTACNGGTCTGTTCTGGGTGNA
NCATTNTCNTGGAAATTTTGCGAANGGCANTCGG
BSK-1A2-5 - forward
CTTTTATTTGCTGAGATATTGTTCTAATCCACTGAGTCAGATTTGGTTGGTCTGAAA
AATTTAACCTGTTGTTAAAAATATTTCTTGGAGGAAGCAGCAGAGGAATAACACTAT
TACTCAAGCATTCACAAAGGGGGCAAAGGAATTCTCCGTTTTCTACATCATAGCTCG
TATGTAAGCGTAATCTCTGTTGCCTTCGCTGTTCTTTAGCTTGAACGGAATCAAAAT
ACCTTTGCCAACAATGGGCATGCATAATGTGCCCACAGCTACTAGTGTGTGTTCCAC
AAGACAGATCAGGGTGCATGAATAATGGATCATATTTTTCTGGATCTTGAATAAATT
TACTTCTGTTTTTTGATAATACAGTTGATCTCTGAACAAATGCTGCCAAGACCATTG
CCCTGCTTTCCACTTTAACTTCTTGCTCCTCTTGACA
BSK-1C7-1 - forward
GTGGCTTGGAGGGGTTAAGAGACTTATCAAAGATCTTGGGGCTAGGTAGTAGAAAAA
CAGAAAAAAAATCAGGTTTTTCAACTGCAGTCAGTACTTTTTTAACAAATTAAAATA
TATCAAATCTCTTTCTCCTAGGTACCTAAAGGCCTAAAAATCCATCAACACAGGGAT
ATATATTAGAAAACCATACCAAGATAAAATGCAAAGGTCAAGAAAATAGAAATGTTA
AAACTCCTTTTGTATGTCATGTATTTCCACAGTTTTGTGGTGAACAAGTATGAATTT
AGGGAACTGGATACTAGAGAGAAAGGAATCATCTCCCTTCACTTGCTAAGGAATTGC
TGGTGCCCTGGGCCACAAGAAGGGTGTGATTTGGGGGGACTGTGTGCAATTAAACAG
GAAAGGAAATAACAGACTTAAAGTATTAAGTCATTCTGATGCTTATCAACAAGAGTA
AAGCACAGCCTAATAAATAAATATATTTGAGAATCTATATTAATCCAGACAGAATGA
CCAAGAGGCTTGATGTCCTGGNAATAACCACATGAAACCTTTTTATTNAAGGACTAC
CACTTATGAAATATGAAAGAATTCCTTANACAAATCCAATCTTANATCTGNATTCTN
AACATTTTCTCCCTTTCCATTTTGAATGCTAATATTAGAAGCATTNTAAAGTAATTT
NGGCCCGCCCATGGCTTACCCTGCAACTCGGACTTTGCAGGCCAGTGGGAGGACTCT
TGAGGCNAANTTTAAAACCNCNNGGCANATTNNCAAANCTGGGCATTTTTAAAANNG
ATNGGAACTTTTTNCCCCCCNANTANAACAATNTTCCNNCCCTTAAACCCNAACCTT
TCCCNGGGTTTTNCCCTAAGGGNCCCTTTTNTTTGAAACCCAAAAGGTNCNTTTGGG
TTNCANGNATTTNAAATTNTTTTNGNCCCNAA
BSK-1E2-A2 - forward
CAGGTTTCTGACCTGGGCTGCAGGGTGAAAGATAGTACTATTAGTTGAAATAGATAA
TACACAGAGAAGAGTAAATTTGGCAAGAACATATTTTGTTTATGTTGAGTTCGTGCC
TGTGAGATACAGGTGGAGGTACCTAGAGACAATTCTGTAGAAGTCTGAAGTTTAGGA
GAGAGGGCAGAGTTGGAGATAAAATTTTAGGTTGTAAGCCTATCATAAATAGTTAAA
ATGGTGAGAATGAGGGAGATTGCTCAGGGCAGTGGTTCACAAATTTTGAGTTTTTCT
GTACAAAAATAAACTTGGAATAAATACCACATGTTCTCACCCATATATGGAAGCTGA
AAAAAAAATGAGCTCATAGAAGTACAGAGTAGAATCTAGAATCATGGTCATTAGAGG
TTGGGAAGGGTAGTGAGGAGAGGAGAATAGGCAGAGGTANCAGATACANAGTTACAG
CTGTTAGGGAGGAAAAAGTTCAGNGCTTTTGNACCATGCNCCCCTGANTNTGNNCAA
ATAATTNAGNCTTNTTTCNACCGCTANAAAAAGCATTTTGAATTTCCCNCCCNAA
BSK-1E2-B - forward
GGCATANNCTCGNTTTGTNANCAGGCTGGATCGAGTGCAGNGATNTNANNTNACTGN
AACCGCCACCTCCCGGGTTNAAGCNATTCCCTGACTCANCCTGTANNCCANTANCTG
GGACTACAGGCNTGCCCCACCTTGCCCGGNTAATTTTTTTTTTTTTNGGATTTTANT
AAAAACGGGGTTTNACCATNTTGGCCANAATCGNCTTGATCTCCTGACCTTCGGATT
ACCCCCACCTNGGCCTCCCAAAGGGNTG
BSK-1G13-A5 - forward
TAAATTTTTTTACTCTCTCTACAAGGTTTTTTCCTAGTGTCCAAAGAGCTGTTCCTC
TTTGGACTAACAGTTAAATTTACAAGGGGATTTAGAGGGTTCTGTGGGCAAATTTAA
AGTTGAACTAAGATTCTATCTTGGACAACCAGCTATCACCAGGCTCGGTAGGTTTGT
CGCCTCTACCTATAAATCTTCCCACTATTTTGCTACATAGACGGGTGTGCTCTTTTA
GCTGTTCTTAGGTAGCTCGTCTGGTTTCCCGGGTCTTAGCTTTGCCTCTCCTTGCAA
AGTTATTTCTAGTTAATTCATTATGCAGAAGGTATAGGGGTTAGTCCTTGCTATATT
ATGCTTGGTTATAATTTTTCATCTTTC
BSK-1G13-B3 - forward
AATCTATCACCCTATAGAAGAACTAATGTTAGTATAACTAACATGAAAACATTCTCC
TCCGCATAAGCCTGCGTCACATTAAAACACTGAACTGACAATTAACAGCCCAATATC
TACAATCAACCAACAAGTCATTATTACCCTCACTGTCAACCCAACACAGGCATGCTC
ATAAGGAAAGGTTAAAAAAAGTAAAAGGAACTCGGCAAATCTTACCCCGCCTGTTTA
CCAAAAACATCACCTCTAGCATCACCAGTATTAGAGGCACCGCCTGCCCAGTGACAC
ATGTTTAACGGCCGCGGTACCCTAACCGTGCAAAGGTAGCATAATCACTTGTTCCTT
AATTAGGGACCTGTATGAATGGCTCCACGAGGGTTCAGCTGTCTCTTACTTTTAACC
AGTGAAATTGACCTGCCCGCGAAGAGGCGGCATAACACAGCAAGACGAGAAGACCCT
ATGGAGCTTTAATTTATTAATGCAAACAGTACCTACAAACCCACAGGTCCTAAACTA
CCAAACCTGATTAAAAATTTCNGTTGGGGCGACCTCGGAGCAGACCCACCTTCGAGC
AGTACATGCTAAGACTTCACCAGTCAAAGCGACTACTATACTCAATTGATCCAATAA
CTTGGNCAACCGGAACAAGTTACCCTTANCGATAACAGCGCAATCCTATTCTAGAGT
CCTTTTAACCATAGGGGTTACCAACCTNAATGTTGGATCAAGGACTTNCCATGGNGC
AACCCGNTNTTAAGGGTCGTTGGTTAACGATAAAGGCCTCCGGAACTGNGTTAAACC
GGNGTAATCCAA
BSK-1G11-A5 - forward
GCATTGGGGTGGGGGTAAGGTGCATCTGTTTGAAAAGTAAACGATAAAATGTGGATT
AAAGTGCCCAGCACAGAGCAGATCCTCAATAAACATTTCATTTCCCACCCACACTCG
CCAGCTCACCCCATCATCCCTTTCCCTTGGTGCCCTCCTTTTTTTTTTATCCTAGTC
ATTCTTCCCTAATCTTCCACTTGAGTGTCAAGCTGACCTTGCTGATGGTGACATTGC
ACCTGGATGTACTATCCAATCTGTGATGACATTCCCTGCTAATAAAAGACAACATAA
CTC
BSK-1G11-B5 - forward
CTAATTACTACCTTTTATTCTAATGTGAACCATGGCCCTGAAAGCTGATAACAAGCT
TGGCTGAGCAGAGGGAACTAGGGGTCGGCAGAAAGGATTATGGGTGGAAAACATTGG
CTCTTCCTTGGGGAGTGATGCTGGGGAAAGGGAAGAGAGTGGCTCAGCCTGCAGGTA
AATAGGCTAGAAAAGCCAAGGCCAAAGGCTGGAGGGGAGAGGACAGTCAGCATGTCC
AGCCTGGGGTCTGGGTGTAGGGTTATCCCTTCTCCCTGTGCCTTCCCATCTCGTCCA
TGAGCCTAGGCCTTGGAGCCTTGTGTTGGAGGCTGCTGTGATGTCAGGAACGGGGAT
CTGCTAGCTTTTGGCCACTTCCTGGGACCTCACGCCCCTGTTGACAGATGGAGATTG
GGCAGCAGGGCCTTGCTGCATTGGTATCTGCTGTTCCACTTGGTTGGCTTGCCAAGG
TGACGAAAGACCAGGCACCANGGTCTCATGGGATGAAGGACAGGGTGGGAAGATGGG
GGAAGGGCTGGGGCTTAAGGGAGCAAGAAAGCTTGTACCTGTGTNGGGCCGGCAGGA
TGTTAAAAACCGCTTTGNTGTTTTAAAATGCGGACTGGGCCCAAATCCTGTTGGGCA
CCCANNCCCAAAANACGGGTCCTCCANTTCCAAGGGANNTTTNGGGGGAACCNNAAN
GGGCTTTTTTCCAGGAANGCCNGTTTTTTNAAACNGGANCCNTGGGCATTC
BSK-1H13 - forward
CGGGATTCCGGTGGCAACGTTGCTGGTGACAGCAAAAATGACCCACCAATGGAAGCA
GCTGGCTTCACTGCTCAGGTGATTATCCTGAACCATCCAGGCCAAATAAGCGCCGGC
TATGCCCCTGTATTGGATTGCCACACGGCTCACATTGCATGCAAGTTTGCTGAGCTG
AAGGAAAAGATTGATCGCCGTTCTGGTAAAAAGCTGGAAGATGGCCCTAAATTCTTG
AAGTCTGGTGATGCTGCCATTGTTGATATGGTTCCTGGCAAGCCCATGTGTGTTGAG
AGCTTCTCAGACTATCCACCTTTGGGTCGCTTTGCTGTTCGTGATATGAGACAGACA
GTTGCGGTGGGTGTCATCAAAGCAGTGGACAAGAAGGCTGCTGGAGCTGGCAAGGTC
ACCAAGTCTGCCCAGAAAGCTCAGAAGGCTAAATGAATATTATCCCTAATACCTGGC
ACCCACTCTTAATCAGTGGTGGAAGAACGTCTCAGAACTGTTGGTTCAATTGGNCAT
TAAGTTTAATAGTAAAAGACTGGGTAATGATACAATGCATCGTAAAACCTTCAGAAG
GAAAGGAGAATGTTTGTGGACCACTTTGGGTTTCCTTTTTGCGTGNGCANTTTTAAG
TATTAGNTTTTAAAACAGNCTTTTAATGGNACACTTGNCCNAAAATTTGCCCAAATT
TTGGAACCCTTTAAAAAGTTAATGGGAAAAAAAAACGGATTCCGGGGGTACCTTCCA
AAACTTTTAAAAANCNGGCCCGCATTTTTTCTGAGGGGTAACNNGTTCCCCATAATT
CCCCCNGGGANAAGCNTNTNNCTTTNGGGACCNTTTTGNANCCCCNTTTTAAGGCCC
CCCNTTTTAACAACCCCCCCCTTGCNTGGACNNANAAANNNCGGNTTTTTATTTTTA
NGAACAAACCNTTNGGTTCNAANCCCTTGGTCNCCCCGGGGGGTNNCNAAAATTTTT
TTCCCCNTTTTTNNGGGGNAAATTNGGGAAATT
BSK-1I2-A2 - forward
CTCCACGAGGGTTCAGCTGTCTCTTACTTTTAACCAGTGAAATTGACCTGCCCGTGA
AGAGGCGGGCATGACACAGCAAGACGAGAAGACCCTATGGAGCTTTAATTTATTAAT
GCAAACAGTACCTAACAAACCCACAGGTCCTAAACTACCAAACCTGCATTAAAAATT
TCGGTTGGGGCGACCTCGGAGCAGAACCCAACCTCCGAGCAGTACATGCTAAGACTT
CACCAGTCAAAGCGAACTACTATACTCAATTGATCCAATAACTTGACCAACGGAACA
AGTTACCCTAGGGATAACAGCGCAATCCTATTCTAGAGTCCATATCAACAATAGGGT
TTACGACCTCGATGTTGGATCAGGACATCCCAATGGTGCAGCCGCTATTAAAGGTTC
GTTTGTTCAACGATTAAAGTCCTACGTGATCTGAGTTCAGACCGGAGT
BSK-1I2-B5 - forward
GCTTTAATATACGCTATTGGAGCTGGAATTACCGCGGCTGCTGGCACCAGACTTGCC
CTCCAATGGATCCTCGTTAAAGGATTTAAAGTGGACTCATTCCAATTACAGGGCCTC
GAAAGAGTCCTGTATTGTTATTTTTCGTCACTACCTCCCCGGGTCGGGAGTGGGTAA
TTTGCGCGCCTGCTGCCTTCCTTGGATGTGGTAGCCGTTTCTCAGGCTCCCTCTCCG
GAATCGAACCCTGATTCCCCGTCACCCGTGGTCACCATGGTAGGCACGGCGACTACC
ATCGAAAGTTGATAGGGCAGACGTTCGAATGGGTCGTCGCCGCACGGGGGGCGTGCG
ATCGGCCCGAGGTTATCTAGAGTCACCAAAGCCGCCGGCGCCCGCCCCCGGCCGGGG
CCGGAGAGGGGCTGACCGGGTTGGTTTTGATCTGATAAATGCACGCATCCCCCCCGC
GAAGGGGGTCAAGCGCCCGTCGGCATGTATTAACTCTAGAATTACCACAGTTATNCA
AGTAGGANANCAGCGAGCGACCAAAGGAACCNTACTGGATTAATGAGCCNTTTCCAG
TTTCACTGTACCGGNCGTGCNANTTAAACATGCATTGGNTTAATCTTTGAGACAAGC
ATATGCTANTGGCANGGTTTTTTTATGGNAAAGATGNTTTATTGGNGGCAGTACTAC
AAGGCATTAATATTGGTNCCCAAAAAAAAACTCGGTNTTATTAAATANTGGGCNTTA
ANACNTAATGAACTTGACCAACNNTTGCTGGATNNCTGANTCCTCCTGGTTTTTGGG
AAAGNAACCCACCACTATTTTTGGCANTCTTTTCNCCACTTGAAAANAAGGGGGTTT
NTNGGNGGCTTANTTCCNNCTTTAANCNGGAATTTTANCCCTNGAANNTTGTTTTCC
GAACTTTTTAAAA
BSK-1L2-2 - forward
AAGGGAAAGATGAAAAATTATAACCAAGCATAATATAGCAAGGACTAACCCCTATAC
CTTCTGCATAATGAATTAACTAGAAATAACTTTGCAAGGAGAGCCAAAGCTAAGACC
CCCGAAACCAGACGAGCTACCTAAGAACAGCTAAAAGAGCACACCCGTCTATGTAGC
AAAATAGTGGGAAGATTTATAGGTAGAGGCGACAAACCTACCGAGCCTGGTGATAGC
TGGTTGTCCAAGATAGAATCTTAGTTCAACTTTAAATTTGCCCACAGAACCCTCTAA
ATCCCCTTGTAAATTTAACTGTTAGTCCAAAGAGGAACAGCTCTTTGGACACTAGGA
AAAAACCTTGTAGAGAGAGTAAAAAATTTACCGCCGATACTGACGGGCTCCAGGAGT
CGTCGCCACCAATCCCAAGGGCGAATTCCAGCACACTGGCGGNCGTTACTAGTGGAT
CCGACTCGGTACCAAGCTTGATGCATAGCTTGAGTATTCTATAGTGCACCTAAATAG
CTTGGCGTAATCATGGNCATACTGTTCTGNGTGAAAATGGTATCCGTNACAATTTCA
CACACATACGAGCCGGAGC
BSK-1A2-5 - reverse
TCAAGAGGAGCAAGAAGTTAAAGTGGAAAGCAGGGCAATGGTCTTGGCAGCATTTGT
TCAGAGATCAACTGTATTATCAAAAAACAGAAGTAAATTTATTCAAGATCCAGAAAA
ATATGATCCATTATTCATGCACCCTGATCTGTCTTGTGGAACACACACTAGTAGCTG
TGGGCACATTATGCATGCCCATTGTTGGCAAAGGTATTTTGATTCCGTTCAAGCTAA
AGAACAGCGAAGGCAACAGAGATTACGCTTACATACGAGCTATGATGTAGAAAACGG
AGAATTCCTTTGCCCCCTTTGTGAATGCTTGAGTAATACTGNTATTCCTCTGCTGCT
TCTCCAAGAAATATTTTTAACAACAGGTTAAATTTTTCAGACCAACCAAATCTGACT
CAGTGGATTAGAACAATATCTCAGCAAATAAAAGCGGAATTCCAGCTGAGCGCCGGC
GCTACCATTACCAGTTGGTCTGGGGCAAAAATAATAATTACCGGGCAGGCCATGTCA
AGG
BSK-1C7-1 - reverse
GAAATGCATTCTTATGTTATACCAAAACACATATTCATAGTAGTTCTATTTATAGTT
GCCCCAAACTAGGTCAATCAAATCTTCAAAAAAAGTAAAATAGTTAATTCATGGTCA
CAAAACATACATATTTCATAATTTCATTTGTATAAACCTCAAAAGCAAAACCAATCT
ATGGTATTTCAAGTCAAGATTGTGGTTACCTTTAAGGGAGAAAATAGCAACTGGGAA
AAGGTATGAGGGGGGATTCTAGGGTGCTGGTAACGATCTGTTTCTTGATTTGGGTGC
TGGCTATATATGTTCACTATTCATTTTTTAAAAATAGACACAGGGTCTCACTATGTT
GCCCAGGCTGGCTTAAACTCTTGGCTCAAGCAGTCCTCCCACCTCGGCCTCCCAAAG
TGCCGAGATTACAGGTGTGAGCCACTGCCCCGGCCGAGATTTACTTTTATAATGACT
CTAATATTTAGCATTCAAAATTGTGAAAGGGGAGAAAGATTCTGAGAAATACAGAAT
CTAAAATGGGATTGNCTAAGTAATCTTTCATATTCATAAGTTGTAGNCTTAAATAAA
AAGGTTCATGTGGTANTACCAGGACATCANCCTCTGGTCATTCTGGCTGGATAATAT
AGATCTCAAATATATTAATTATTAGNCGGGCTTTACTCTGGTGATAANACTCNNAAN
GCTAATACTTTAAGNTGGNATTCCTTTCTGGTAATGGNACAGTCCCCAANTAAACCN
TTTTGNGCCANGGNCCACATTCNTACAGGGAAGGGAAAAANCCTTTTNTTAGNTCAA
TCCTAATCACTTTTCCCCAAATGGGGANNCTGCNTCCAAGGNNTAANNTTTTTTTNG
CCTTNNTTTNATNGGNGGNTTAAAAAANCCCCGGNNNGGTTTNGCCTTNGCCCGNAA
AANTTTTTTTTTNNAAAAANNCNNGTNTAAACCNTTTTTTTTTAAAAGGGANC
BSK-1E2-A2 - reverse
GTGTTGTGAATATTCAAAATCCTCTCTTCTAGCTGTTTGAAAATATACACTAAATTA
TTGTGAGCAATATTCAGGCTACCATGCTACAGAGCACTGAACTTTTTCCTCCCTAAC
AGCTGTAACTTTGTATCTGTTACCTCTGCCTATTCTCCTCTCCTCACTACCCTTCCC
AACCTCTAATGACCATGATTCTAGATTCTACTCTGTACTTCTATGAGCTCATTTTTT
TTTCAGCTTCCATATATGGGTGAGAACATGTGGTATTTATTCCAAGTTTATTTTTGT
ACACAAAAACTCAAAATTTGTGAACCACTGCCTGAGCAATCTCCCTCATTCTCACCA
TTTTAACTATTTATGATAGGCTTACAACCTAAAATTTTATCTCCAACTCTGCCTCTC
TCCTAAACTTCAGACTTCTACAGAATTGCTCTAGGTACCTTCACCTGTATCTACAGG
CACGAACTCACATAAACAAAATATGTTCTTCCCAAATTTACTCTTCTCTCTGGATAA
CTATTTCAACTAATAGTCTATCTTTCACCCTGGAGCCAGTCAGAAACCTG
BSK-1E2-B2 - reverse
CAGCACTTTGGGAGGCCGAGGTGGGGGTAATCACAAGGTCAGGAGATCAAGACCATC
CTGGCCAACATGGTGAAACCCCGTCTNTACTAAAATCCAAAAAAAAAAAAAATTAGC
CGGGCAAGGTGGCGCATGCCTGTAGTCCCAGCTACTGGACTACAGGCTGAGTCAGGG
AATCGCTTGAACCCCGGAGGTGGCGGTTGCAGTGAGCTGAGATCACTGCACTCCATC
CAGCCTGCTGACAGAGCGAGACTATGCCTCAAAAAAAAAANANAAAAANAANANAAA
ANAAAACNNAAAAAANAAAAAANAAAAACCAGTTGANCGCCGGNCGNTACCATTCCA
GGGGGTCTCCGGTCAAAAATANTAATANCCGGGCAGGCCATNTCAAGGGCGAATTNT
GCAGATATCCAT
BSK-1G13-A5 - reverse
GAAAGATGAAAAATTATAACCAAGCATAATATAGCAAGGACTAACCCCTATACCTTC
TGCATAATGAATTAACTAGAAATAACTTTGCAAGGAGAGCCAAAGCTAAGACCCCCG
AAACCAGACGAGCTACCTAAGAACACCTAAAAGAGCACACCCGTCTATGTAGCAAAA
TAGTGGGAAGATTTATAGGTAGAGGCGACAAACCTACCGAGCCTGGTGATAGCTGGT
TGTCCAAGATAGAATCTTACTTCAACTTTAAATTTCCCCACAGAACCCTCTAAATCC
CCTTGTAAATTTAACTCTTAGTCCAAAGAGGAACAGCTCTTTGGACACTAGGAAAAA
ACCTTGTAGAGAGAGTAAAAAAATTTA
BSK-1G11-A5 - reverse
GAGTTATGTTGTCTTTTATTAGCAGCGAATGTCATCACAGATTGGATAGTACATCCA
GGTGCAATGTCACCATCAGCAAGGTCAGCTTGACACTCAAGTGGAAGATTAGGGAAG
AATGACTAGGATAAAAAAAAAAGGAGGGCACCAAGGGAAAGGGATGATGGGGTGAGC
TGGCGAGTGTGGGTGGGAAATGAAATGTTTATTGAGGATCTGCTCTGTGCTGGGCAC
TTTAATCCACATTTTATCGTTTACTTTTCAAACAGATGCACCTTACCCCCACCCCAA
TGC
BSK-1G11-B5 - reverse
CACAGGAGGAGAAGCAGGAGCTGTCGGGAAGATCAGAAGCCAGTCATGGATGACCAG
CGCGACCTTATCTCCAACAATGAGCAACTGCCCATGCTGGGCCGGCGCCCTGGGGCC
CCGGAGAGCAAGTGCAGCCGCGGAGCCCTGTACACAGGCTTTTCCATCCTGGTGACT
CTGCTCCTCGCTGGCCAGGCCACCACCGCCTACTTCCTGTACCAGCAGCAGGGCCGG
CTGGACAAACTGACAGTCACCTCCCACAACCTGCAGCTGGAGAACCTGCGCATGAAG
CTTCCCAAGCCTCCCAAGCCTGTGAGCAAGATGCGCATGGCCACCCCGCTGCTGATG
CAGGCGCTGCCATGGGAGCCCTGCCCAGGGGCCCATGCAGAATGCCACCAAGTATGG
CAACATGACAGAGGACCATGTGATGCACCTGCTCCAGAATGCTGACCCCCTGAAGGT
GTACCCGCCACTGAAGGGGAGCTTCCCGGAGAACCTGAGACACCTTAAGAACACCAT
GGAGACCATAGACTGGAAGGTCTTTGANAGCTGGATGCACCATTTGGCTTCTGTTGA
AATGAGCANGCACTTCTTTGGACAAAAGCCCACTTGACGCTTCANCGAAGAGTCACT
TGGAACTGGAGGACCGTCTTTNGGCTGGTGTGACCAACAGGATCTGGGCCAATNCCC
ATTGAAACAACANAAGCGGCTTTAAAATCTTGCGGGCCCANAAAGTTCAANTTTNTT
GGTTCCTTAGGCCCAANCCTTCCCAATTTTCNACTTGGNCCTAATCCATGAAAACTG
GNGCNNGGTNTTTNTNANCCTTGGNAAGAAAAACAATTGGAACANCGATAACATGCN
NAAGGCCTNGTCGCCAAATTCTTTTTAANANGGGCTAGGGCCCNAAANGGCAAAATT
NAAAAACCCTNNTGAATAAAANATTTAANAAAGGTNANGGTTNGTNTTGNCAAATGG
AANGCCCNGNAAGGGAACCTCCCCNACCNANNGGANNTGNANGNTTCCNCAANTGGC
TT
BSK-1H13 - reverse
CGGAGTTCCGGGTATCTGGGCTCCAGGCAGAAGCACAGCCTCCCCGACCTGCCCTAC
GACTACGGCGCCCTGGAACCTCACATCAACGCGCAGATCATGCAGCTGCACCACAGC
AAGCACCACGCGGCCTACGTGAACAACCTGAACGTCACCGAGGAGAAGTACCAGGAG
GCGTTGGCCAAGGGAGATGTTACAGCCCAGATAGCTCTTCAGCCTGCACTGAAGTTC
AATGGTGGTGGTCATATCAATCATAGCATTTTCTGGACAAACCTCAGCCCTAACGGT
GGTGGAGAACCCAAAGGGGAGTTGCTGGAAGCCATCAAACGTGACTTTGGTTCCTTT
GACAAGTTTAAGGAGAAGCTGACGGCTGCATCTGTTGGTGTCCAAGGCTCAGGTTGG
GGTTGGCTTGGTTTCAATAAGGAACGGGGACACTTACAAATTGCTGCTTGTCCAAAT
CAGGATCCACTGCAAGGAACAACAGGCCTTATTCCACTGCTGGGGATTGATGTGTGG
GAGCACGCTTACTACCTTCAGTATAAAAATGTCAGGCCTGATTATCTAAAAGCTATT
TGGAATGTAATCAACCGGAATTCCGTTTTTTTTTTTCTCATTTAACTTTTTTAATGC
GCTCAAAATTCTGNGACAAANTTTTGGCAAGTGTTTCCATTAAAAAGTNTGATTTAA
AACTAATACTTAAAATTGCNCACCCNAAAANGGAAAACCAAGTGGTCCCAAACATTC
TCTTTCTTNTAAGGTTACANGCNTGGTNTATTAACCACTTTTCTCTAACTTAANGCC
ATTGAACAACATTTTAAACGTTTCNCCNGTTAAAANGGGGGGNGGTTNGGGNAAATN
NTTACCTTTGACTTTTGGNNAANTTGGGACTTCNNTTCNAACTTTTTCCNGGTTTNA
CCCCCCAANGNGGTTTTC
BSK-1I2-A2 - reverse
ACTCCGGTCTGAACTCAGATCACGTAGGACTTTAATCGTTGAACAAACGAACCTTTA
ATAGCGGCTGCACCATTGGGATGTCCTGATCCAACATCGAGGTCGTAAACCCTATTG
TTGATATGGACTCTAGAATAGGATTGCGCTGTTATCCCTAGGGTAACTTGTTCCGTT
GGTCAAGTTATTGGATCAATTGAGTATAGTAGTTCGCTTTGACTGGTGAAGTCTTAG
CATGTACTGCTCGGAGGTTGGGTTCTGCTCCGAGGTCGCCCCAACCGAAATTTTTAA
TGCAGGTTTGGTAGTTTAGGACCTGTGGGTTTGTTAGGTACTGTTTGCATTAATAAA
TTAAAGCTCCATAGCGTCTTCTCGTCTTGCTGTGTCATGCCCGCTCTTCACGGCAGG
TCAATTTCACTGGTTAAAAGTAAGAGACAGCTGAACCCTCGTGGA
BSK-1I2-B5 - reverse
CATTTTCGTTGGTGGTGTTCAGTTGTGGCGGTTGCTGGTCAGTAACAGCCAAGATGC
TGCGGAATCTGCTGGCTCTTCGTCAGATTGGGCAGAGGACGATAAGCACTGCTTCCC
GCAGGCATTTTAAAAATAAAGTTCCGGAGAAGCAAAAACTGTTCCAGGAGGATGATG
AAATTCCACTGTATCTAAAGGGTGGGGTAGCTGATGCCCTCCTGTATAGAGCCACCA
TGATTCTTACAGTTGGTGGAACAGCATATGCCATATATGAGCTGGCTGTGGCTTCAT
TTCCCAAGAAGCAGGAGTGACTTCAGTCATCCCAGCAATCGCTTGCTTCAGTTTCAT
TCAGCTCTCTATGGACCAGTAATCTGATAAATAACCGAGCTCTTCTTTGGGGATCAA
TATTTATTGACTTGTAGTAACTGCCACCAATAAAGCAGTCTTTACCATAAAAAAAAA
CCTGCCAGTAGCATATGCTTGNCTCAAAGATTAAGCCATGCATGTCTAAGTACGCAC
GGCCGGTACAGTGAAACTGCGAATGGCTCATTAAATCAGNTATTGGGTCCTTTGGTC
GCTNGCTCCTCTCCTACTTGGATACTGNGGTAATTCTAAACTAATACATGCCGACGG
GCGCTTACCCCTTNGCGGGGGGGATCCTGCATTTATANATCAAACCAACCCGGNAAG
CCTTTTCGGCCCGGCCGGGGGCGGCCNCCGGNGNTTTTGGNGACTTTAANAACCTTN
GGCCCAANGACCCCCCCNNGGGGGGA
BSK-1L2-2 - reverse
TTTTTACTCTCTCTACAAGGTTTTTTCCTAGTGTCCAAAGAGCTGTTCCTCTTTGGA
CTAACAGTTAAATTTACAAGGGGATTTAGAGGGTTCTGTGGGCAAATTTAAAGTTGA
ACTAAGATTCTATCTTGGACAACCAGCTATCACCAGGCTCGGTAGGTTTGTCGCCTC
TACCTATAAATCTTCCCACTATTTTGCTACATAGACGGGTGTGCTCTTTTAGCTGTT
CTTAGGTAGCTCGTCTGGTTTCGGGGGTCTTAGCTTTGGCTCTCCTTGCAAAGTTAT
TTCTAGTTAATTCATTATGCAGAAGGTATAGGGGTTAGTCCTTGCTATATTATGCTT
GGTTATAATTTTTCATCTTTCCCTTGCCGAAATTCC
BSK-1K9-A3 - forward
ATGTAAGTAAGTGTATTATGGCCAGTTAAGGTAGGCACTATAAAAATAGGCCGAAAA
GTTTAGAATATTCCTTTTTTACTGTAGTCTGTTTTTTAAAATTTGAAACTTGTTAGA
GAGTTTGGAAAACAGTCTTCTTCCTCCCACTCCACTTCCTGCCAAAAAAGAGGGGAA
GCACAATCGTCTTCAAAAAAGGTGATAAAGTAAATGCATATTATAAAATATTTTAAA
CTTTTGTGTGTGTGGTTTCACGTACAGGAAATGAACATGCAAATTCTTAGAAACTGT
TGTCACTGTGTTTCTGAAATGCTAAAAAAAATTATGCTTTGAGCTACCTGCTGCTTA
TAATTCCTTTCCCTGAATAGGTAGGTTTTTATAGTTAACAAATTTTAAATGTAAGTT
GATTTTGATAGTACTATTTCATTATGCAATCTGGAGAGGAGAGAAGTGTTTTTCATA
AAGTGGATATTAATTACAACTTTNAAAAGCCAATCAGTAAACATTCATTGATCTTGN
AATAACTGNGACCCTAATTAAAAGGGTGCTAGGCTTGTATGCTTGGAAATATTTGAA
ATTTTTATTNTTAAAACTGGG
BSK-1K9-A3 - reverse
CAGATTTTAAAGAATAAAAAAATTTCAAATATTTTCCAGACATAACAGCCTAGCAAC
CATTTTAATTAGGTGTCACAGTTAATTACAAGATCAATGAATGTTTACTGATTGGCN
TTTTAAAGTTGTAATTAATATCCACTTTATGAAAAACACTCTCTCCTCTCCAGATTG
CATAATGAAATACTACTATCAAAATCAACTACATTTAAAATTTGTTAACTATAAAAA
CCTACCTATTCAGGGAAAGGAATTATAAGCAGCAGGTAGCTCAAAGCATAATTTTTT
TTAGCATTTCAGAAACACAGTGACAACAGTTTCTAAGAATTTGCATGTTCATTTCCT
GTACGTGAAACCACACACACAAAAGTTTAAAATATTTTATAATATGCATTTACTTTA
TCACCTTTTTTGAAGACCATTGNGCTTNCCCTCTTTTTTGGCCAGGAAGTGGGAGTG
GGAGGAAGAANACTGTTTTCCAACTCTTAACAGGTTCAAATTTTAAAAAACAGACTA
CNGTAAAAANGGATATTCTAAACTTTCGGNCTATTTTATAGGCCTACCTAACTGGCC
TAATCCTTACTACATNGGATTCCNCTGANCGCCG
BSK-2C5-C3 - forward
AGAATCTGGTGACTTCAGTTGAGCCCCCAGCAGAGGTGACTCCATCAGAGAGCAGTG
AGAGCATCTCCCTCGTGACACAGATCGCCAACCCGGCCACTGCACCTGAGGCACGAG
TGCTACCCAAGGACCTGTCCCAAAAGCTGCTAGAGGCATCCTTGGAGGAACAGGGCC
TGGCTGTGGATGTGGGTGAGACTGGACCCTCACCCCCTATTCACTCCAAGCCCCTAA
CGCCTGCTGGCCACACCGGCGGCCCAGAGCCCAGGCCTCCAGCCAGAGTAGAGACTC
TGAGGGAGGAGGCGCCCACAGACTTACGGGTGTTTGAGCTGAACTCGGATAGTGGGA
AGTCTACACCCTTCAACAATGGAAAGAAAGGCTCAAGCACGGACATTAATGAGGACT
GGGAAAAAGACTTTGACTTGGACATGACTGAANAGGAAGTGCANATCGCACTTTCCA
AGTGGATGCCTNCNGGGAGCTNGAAAATTAAAATGGGAAGACTGGGAATGAGGGACC
NNAAGGAGCANTTCCCCCCCATGGGATNTTTTGCTTCCTNCTNGNTTAANCCANCCT
GGATGAATGAAAATGTTCCCCAAATTCTTTGCAACCAAACTTTGGCACAAATTTGGG
GGTNCTTGTTGGCCTTTTGGNCTTTGTTNACCNGGAAGGGTTTTANTCCGGCCAAAA
TTTTATTTGCCNCATTGGNGACCCNGGGGAGGAACTNTCTCTNCCNAAAACGGTTTT
TNTNAACCNTGTTCTTANGATNTTTTGAACCNAGGAATTTNCCTTTCTGTNAAAAAA
NAACCCNNTTTTNNGAANNGNAANTNTTNNTTTTNNNGGGGGGGGNNCCCTCCTTGT
AAAAG
BSK-2C5-C3 - reverse
AAAGCAAGGAGGTGGGTCAGGGTTTGGTCTCTGGATTCTGAACCCCAAAGGAGCCTT
TCCAGGAATGGAAAATGCCTGGGAGGGGGAGAGTCCCAAGAGAGGCAAATTTCCCAG
AGATAAGTGCCTCTTACCCACTGGGATAGGAACCAAAATGTGTTCACTGTCCCTGTT
TAGCCAAGGGTAGGTGGCATGGCCCTCCCTGCCTGCTTATGTATGGACAGAGTATGT
TGTCTCAGCTTCCTCCGAGAGAGACTGGTGGTTTAGCTTCTGTCTACACAGGCAGAA
GGGCTAGAACTATCCCTTGGGACTTTCCAGCAGGAGTCCTCANGAACAGTGGGTGTT
CANCAGAAAAACACANGCTCTTCTGGTGAGGAGGATAGGTTTCCTCTTCCTTGGGTC
ATCCTATTGTTGGCACAAGTCAAAGTTTTTGGCCGGGATTTANAAAGCCCCTTCCAG
GTGTGAGCANAAGCCCAAAANGGCCANCAGGGAACCCCAAATTGTCCCAAACTTTTG
TTGCAAAAGANATTTGGGGGAACATTNTCANTCATTCAGGCTGGCTTANACAACCAN
GGANGCAAAAATGCCTTGGTGGGGGAGNTGTTCCTTTGGNTTCCTTATTCCANNNCT
TCCATTTTAATTTTNAACTTCCCGGAGNATCCCTTTTGNAAGNCCNTTTCNCCTCTT
TTNATCATTTNCAANNAAANNTTTTTCCANCCTACTNTNTCCCGCTTAACCTTTTTT
NTTNTTGGNGGGGGNNATTCCCTTTCNNTTANTTAAAAACCCNANTTNNGGCCCNCN
CCTCAANTTTTTTTNTTAACCTNNNTTTGNCCCCNTGNCCNANCNTNGGCTNGATAA
ATNGGGNGGGNNATTTNCCCATNCNACANNCTNTTTTANNATTT
BSK-2G9-D3 - forward
ATCCCAGGAAAATTTGGAGGAACAGCTGCTCTCCACTGGCCTGCTCCTGCAAGAATG
CCCTGGAGCTTCTGAAGAAGGATCTATATTTACCTTATAGGGCCTTAAGTCCTGGGA
TGGAACTATATACTTTGGCCGCGATGATGTGGCTTTGAAGAACTTTGCCAAATACTT
TCTTCACCAATCTCATGAGGAGAGGGAACATGCTGAGAAACTGATGAAGCTGCAGAA
CCAACGAGGTGGCCGAATCTTCCTTCAGGATATCAAGAAACCAGACTGTGATTGACT
GNGAGAGCCGGGCTGAATGCAATGGAGTGTGCATTACCATTTNGGAAAAAAAATGTG
AATCANTCACTTACTGGGACCTGNACAACTNGCCAACTGACAAAAATGACNCCCATT
TGTGTGACTTTATTNGANANCATTACCTGGAATGANCCGGTGAAAAACCCTTNAAAG
AANTTTGNCTGACCACATTTCNCAAAATTNCACANNAATNGNANGCCCCCCGNATAT
GGCTTGNATAGGAATANTCNTTTNTGACAAGCACACCCT
BSK-2G9-D3 - reverse
GGGTACCAAATTTCTTTATTTGAAGGAATGGTACAAATCAAAGAACTTAAGTGGATG
TTTTGGTACAACTTATAGAAAAGGTAAAGGAAACCCCAACATGCATGCACTGCCTTC
GTGACCAGGGAAGTCACCCCACGGCTATGGGGAAATTAGCCCGAGGCTTANCTTTCA
TTATCACTGTCTCCCAGGGTGTGCTTGTCAAAGAAATATTCCGCCAAGCCANATTCG
GGCCCTCCCATCTTCCGCAAGTTGGTCACCGTGGTCACCCAATTCTTTGATGGCTTT
CACCTGCTCATTCANGNAATGNGNCTCAATGAAGTCACACAAATGGCCGNCATTTTT
CTCAGTGGCCACTTTGGGCANNTNCAGTANTCACTGATTNACATTTTTTTTCCAATG
GAATGNACACTTCATTGNATTNANCCNNTTTTCCANCATTAAAANNTGGGTTNTTGA
TATNCTNAAGAAAAATNGGCCCCC
BSK-2K13-A4 - forward
AGAAATTGAAACCTGGCGCAATAGATATAGTACCGCAAGGGAAAGATGAAAAATTAT
AACCAAGCATAATATAGCAAGGACTAACCCCTATACCTTCTGCATAATGAATTAACT
AGAAATAACTTTGCAAGGAGAGCCAAAGCTAAGACCCCCGAAACCAGACGAGCTACC
TAAGAACAGCTAAGAGAGCACACCCGTCTATGTAGCAAAATAGTGGGAAGATTTATA
GCTAGAGGCGACAAACCTACCGAGCCTCGTGATACCTCGTTGTCCAAGATACAATCT
TAGTTCAACTTTAAATTTGCCCACAGAACCCTCTAAATCCCCTTGTAAATTTAACTG
TTAAGTCCAAAGAGGAACAGCTCTTTGGACACTAGGAAAAAACCTTGTAGAGAAGAA
GT
BSK-2K13-A4 - reverse
GTTAAATTTTTTACTCTCTCTACAAGGTTTTTTCCTAGTCTCCAAAGAGCTGTTCCT
CTTTGGACTAACAGTTAAATTTACAAGGGGATTTAGAGGGTTCTGTGGGCAAATTTA
AAGTTGAACTAAGATTCTATCTTGGACAACCAGCTATCACCAGGCTCGGTAGGTTTC
TCGCCTCTACCTATAAATCTTCCCACTATTTTGCTACATAGACGGGTGTGCTCTCTT
AGCTGTTCTTACGTAGCTCGTCTGGTTTCGGGGGTCTTAGCTTTGGCTCTCCTTGCA
AAGTTATTTCTAGTTAATTCATTATGCAGAAGGTATACGGGTTAAGTCCTTCCTATA
TTATGCTTGGGNTATAATTTTTCATCTTTCCCTTGCGGNACTATATCTATTGCCCCA
GGTTTCAATTTCT
BSK-2K13-C2 - forward
CAAACCCACTCCACCTTACTACCAGACAACCTTAGCCAAACCATTTACCCAAATAAA
GTATAGGCGATAGAAATTGAAACCTGGCGCAATAGATATAGTACCCCAAGGGAAAGA
TGAAAAATTATAGCCAAGCATAATATAGCAAGGACTAACCCCTATACCTTCTGCATA
ATGAATTAACTAGAAATAACTTTGCAAGGAGAGCCAAAGCTAAGACCCCCGAAACCA
GACGAGCTACCTAAGAACAGCTAAAAGAGCACACCCGTCTATGTAGCAAAATAGTGG
GAAGATTTATAGGTAGAGGCGACAAACCTACCGAGCCTGGTGATAGCTGGTTGTCCA
AGATAGAATCTTAGTTCAACTTTAAATTTGCCCACAGAACCCTCTAAATCCCCTTCT
AAATTTAACTGTTAGTCCAAAGAGGAACAGCTCTTTGGACACTAGGAAAAAACCTTG
TACAGAGAGTAAAAAATTAACACCCATAGTAGGCCTAAAAC
BSK-2K13-C2 - reverse
GCTTTTAGGCCTACTATGGGTGTTAAATTTTTTACTCTCTCTACAAGGTTTTTTCCT
AGTGTCCAAAGAGCTGTTCCTCTTTGGACTAACACTTAAATTTACAAGGGGATTTAG
AGGGTTCTGTGGCCAAATTTAAAGTTGAACTAAGATTCTATCTTGGACAACCAGCTA
TCACCAGGCTCGGTAGGTTTGTCCCCTCTACCTATAAATCTTCCCACTATTTTGCTA
CATACACGGGTGTGCTCTTTTAGCTGTTCTTAGGTAGCTCCTCTGGTTTCGGGGGTC
TTAGCTTTGGCTCTCCTTGCAAAGTTATTTCTACTTAATTCATTATGCAGAAGGTAT
AGCGGTTAGTCCTTGCTATATTATGCTTGGCTATAATTTTTCATCTTTCCCTTCCGG
NACTATATCTATTGCGCCAGGTTTCAATTTCTATCGCCTATACTTTATTTGGGTAAA
TGGTTGGCTAACGNTGCTGGTANTAACGNGGAGTGGGTTTG
BSK-1E15 - forward
AGATCCTTATGCCCGAGTTCCGGTACAGGAACGTCGGTCATCCAGATGCCCTCTTCC
GCTTTCAGTTTGGATAACGCTTTCATCTCACATCCTCAGGCGATAACGCCCAGTTGT
TTACCAATACGCGTAAATGCTTCTACTGCACGCGTAATTTGCTCAGGGGTATGCGCC
GCAGACATCTGCGTACGAATACGCGCCTGACCTTTCGGAACGACCGCATAGAAGAAA
CCGGTAACGTAAATGCCCTCTTTTTGCAGCTCACGGGCAAATTTCTGCGCCACTACC
GCATCACCAAGCATGACCGGAATAATGGCGTGATCGGTTCCGCAGGGTAAAGCCCGC
CGNCGACATTTCCTCACGGAACTGACGCGCGTTCGCCACAGACGGTCACGCAGTTCG
CTGCCCGCTTCGACCATCTTCAGTACTTTGATGGACGCCGNAACAATGGNCGGTGCC
AGCGAATTTGGAGAACANGTACNGACCAANAACCTTGGCGCAAGCCACTCAANCACT
TTTTTTGCGCGCCCGCGGNATAACCCCCCAGAAGCCCCGGNCCAANGCTTTTACCAA
GCGTACCCGGNGATAATATTTGAACCCGGGCCATTAANATTGCAANNTTTATTGGGA
ACCNCGAACATTTTTAACCGNCAAAAACCAACCCCNTNGGAAATNTTNGCCNCCAAT
TCCCANGGGGGAAATTTTNGNAAATTCNTTNAAACTGGGGGGCCGTTTAACATGCCT
TTTAANGGGCCCAATTNNCCCNTTANGGGGCGNTTACAAATNACTNGGCCGGNNTTT
TNAAACNNNNGAATNGGGNAAACCCGGGGGTTCCCAACTTAAA
BSK-1H13 - reverse
CATTGATTGAATAGTTATAAAGATGTTATAGTAAATTTATTTTATTTTAGATATTAA
ATGATGTTTTATTAGATAAATTTCAATCAGGGTTTTTAGATTAAACAAACAAACAAT
TGGGTACCCAGTTAAATTTTCATTTCAGATAAACAACAAATAATTTTTTAGTATAAG
TACATTATTGTTTATCTGAAATTTTAATTGAACTAACAATCCTAGTTTGATACTCCC
AGTCTTGTCATTGCCAGCTGTCTTGGTAGTGCTGTGTTGAATTACGGAATAATGAGT
TAGAACTATTAAAACAGCCAAAACTCCACAGTCAATATTAGTAATTTCTTGCTGGTT
GAAACTTGTTTATTATGTCAAATAGATTCTTATAATATTATTTAAATGACTGCATTT
TTAAATACAAGGCTTTATATTTTTAACTTTAAGATGTTTTTATGTGCTCTNCAAATT
TTTTTTACTGGTTCTGATTGNATGGAAATATAAAAGTAAATATGAAACATTTAAAAT
ATAATTGGTGGGGCATTTTTAATTAAGNTTGGTTTATTTAAGNTTAAGGTAATTCCA
TGCTGGGGTTCANTAGAACATTCCGAATCTGGATCTGNGGNTCCAGCAGATATTCCN
NANTACAAATTANCTTCAAGTCCCCTTCTGGACCAAAAAGGTNACCACCAANGANGG
GAGGAATNAAGGGGAA
BSK-1F14 - reverse
CCAGNTGACCNCCGGNCGTTACCNTTACCAGTNGGTNTGGNGTNAAAAATAATANTA
ACCGGNCAGGCCNTNTNANGGGCAAATTNTGNAAATNTCCNTNANANTGGCGGCCGT
TCNANCNTGCNTTTAAAGGGCCNANTTCNCCNTATAGGGAGTCGTNTTANANTTNAN
TGGCCGTNGTTTNANAACGTCGNNANTGGNAAAACCNTGGNGTTACCCAA
BSK-1H13 - forward
CGGTATTCCGAAAAAATGTTTCCAACTCCGCTGAAATGTTGCTGAAAAGCATGGTGC
TGGTAACAGTTCAACAATCCGTGGCTGCTCATTCTTGCCTACTTTACTCTCCCACTG
AAGCAGGTTAGCGTTGAAGGTGGTATGGAAAAGCCTGCATGCCTCTTCAATTCTTTT
GTTTCTTCTCCTTCCCCCTCCCCCTACCTCCTTCCCCTCACTCCTCCCCTCCTTCGC
TCGCTCAACCTCTTTTGTTCAGTATGTGTAACTTGAAGCTAATTTGTACTACTGGAT
ATCTGACTGGAGCCACAGATACAGAATCTGTATTGTTCTTACTGAAACACAGCATGG
AATTAACATTAAACTTAAATAAAACAAACCTAAATTAAAAATGCCCAACAAATTATA
TTTTAAATGTTTCATATTTACTTTTATATTTCCATACAATCAGAAACAGTAAAAAAA
ATTTGGAGAGCACATAAAAACATCTTAAAGTTAAAAATATAAAGCCTTGTATTTAAA
AATGCACTCATTTAAATAATATTATAAGAATCTATTTGNACATAATAAACAAGTTTC
AACCAGCAAGAAATTACTAATATTGACTGTGGAGTTTTGGCTGGTTAATAGTTCTAA
CTCANTATTCCGTAATCAACACAAGCACTACCAACACAAGNTGGCAATGACAAGAAT
GGGAAGTNTCAAACTAGGATGGTAAGTCAATTAAAANTTCAGATAACCATAATGNAC
TTATACTAAAAAATTATTTTGGGGGTTATTTGAAAANGAAAATTAACTGGGGGNCCC
AATTGGTTGGTTGGGTAAATTTAAAACCCNGGTTGGAAATTATCTAATAAACNTTCN
TTNAATACTNAAAAAAAATAAATTNCCTTACCACTTTTTACCNTTTCATNAAGGGGG
AATTCNATTAACCCCGNGGTTNCATTTNCAATGGGGTGGGGGGC
BSK-1E3 - forward
GAGGCNCAGGTGGGGGTNNTTACANNGTNATGATGATTAATNACCATTCTGNCCAAC
ATGGTNAANCCCNGTNTCTACTAAAATCCAAAAANNNNAAAATTAGCCGGNCAAGGT
GGNGCATGCCTGTAGTCCCAGCTACTGGACTACAGGCTGANTNAGGGAATCCCTTGA
ACCCGGNAGGTGGCGGTTGCAGNGANCTGAGATCACTGCACTCNATCCAGNCTGCTG
ACANATCNAGACTATGCCTCAAAAAANGGGGTTTAACCATNTTGNCCNAAAAGGNNT
TNANANCCTAANCTTGNNAAAACCCCCNTGATGGCCGTTC
BSK-1F14 - forward
CCNANNCTGACGGGNTCNANNANTNGNCCCCNCCAATCCCANGGGCAAATTCCANCN
NNCTGGNGGCCGTTACTAGGGGANCCNANCTNGGNNCCAANNTTGANNCANANNTNG
NGTNTTNNANAGGGGCNCCNAAANANNTNGGNGNAANCANGGNCANANCTGTTNCCT
GGGGAAAATTGTNNTCCNNTNANAATTCCNCNCAANNTACNACCCGGAANCNTAAAG
GGTAAA
BSK- 1E15 - forward
GGTTCCCATGAATACTGCGATGTGATGGGCCGGGTCGATATTATCACCGGTACGCTT
GGTAAAGCGCTGGGCGGGGCTTCTGGTGGTTATACCGCGGCGCGCAAAGAAGTGGTT
GAGTGGCTGCGCCAGCGTTCTCGTCCGTACCTGTTCTCCAACTCGCTGGCACCGGCC
ATTGTTGCCGCGTCCATCAAAGTACTGGAGATGGTCGAAGCGGGCAGCGAACTGCGT
GACCGTCTGTGGGCGAACGCGCGTCAGTTCCGTGAGCAAATGTCGGCGGCGGGCTTT
ACCCTGGCGGGAGCCGATCACGCCATTATTCCGGTCATGCTTGGTGATGCGGTAGTG
GCGCAGAAATTTGCCCGTGAGCTGCAAAAAGAGGGCATTTACGTTACCGGTTTCTTC
TATCCGGTCGTTCCGAAAGGTCAGGCGCGTATTCGTACCCAGATGTCTGCGGCGCAT
ACCCCTGACAAATTACGCGTGCAGTAGAAGCATTTACGCGTATTGGTAAACAACTGG
GCCGTTATCGCCTGAGGATGTGAGATGAAAGCGTTATCCAAACTGAAAAGCGGAAGA
GGCATTTTGGATGACCGACGTTCTGTACCGGAACTCGGCATAACGAATCTGGTTGAT
TAAAAGTCCGTAAACAGCCATTNTGCGGGAATGACGTTCACATTTATAACTGGGGAT
AAGTCTNGCNCCAATNCCAAGG
BSK-1A11-A3 - reverse
CCGGCCCGTCTCGCCCGCCGCGCCGGCGAGGTGGAGCACGAGCGCACGTGTTAGGAC
CCGAAAGATGGTGAACTATGCCTGGGCAGGGCGAAGCCAGAGGAAACTCTGGTGGAG
GTCCGTAGCGGTCCTGACGTCCAAATCGGTCGTCCGACCTGGGTATAGGGGCGAAAG
ACTAATCGAACCATCTAGTAGCTGGTTCCCTCCGAAGTTTCCCTCAGGATAGCTGGC
GCTCTCGCAGACCCGACGCACCCCCGCCACGCAGTTTTATCCGGTAAAGCCAATGAT
TAGAGGTCTTGGGGCCGAAACGATCTCAACCTATTCTCAAACTTTAAATGGGTAAAG
AAGCCCGGCTCGCTGGCGTGGAGCCGGCGTGGAATGCNANTGCCTAATGGGCCACTT
TTGGTAAGCANAACTGGCGCTTGGGGATGAACCGAACGCCGGGTTAAGGGGCCCGAT
GCCGACCTCAT
BSK-1D8-B3 - forward
AAGGAATCGTATCGTATGTCCGCTATCCAGAACCTCCACTCTTTCGACCCCTTTGCT
GATGCAAGTAAGGGTGATGACCTGCTTCCTGCTGGCACTGAGGATTATATCCATATA
AGAATTCAACAGAGAAACGGCAGGAAGACCCTTACTACTGTCCAAGGGATCGCTGAT
GATTACGATAAAAAGAAACTAGTGAAGGCGTTTAAGAAAAAGTTTGCCTGCAATGGT
ACTGTAATTGAGCATCCGGAATATGGAGAAGTAATTCAGCTACAGGGTGACCAACGC
AAGAACATATGCCAGTTCCTCGTAGAGATTGGACTGGCTAAGGACGATCAGCTGAAG
GTTCATGGGTTTTAAGTGCTTGTGGCTCACTGAAGCTTAAGTGAGGATTTCCTTGCA
ATGAGTAGAATTTCCCTTCTCTCCCTTGTCACAGGTTTAAAAACCTCCAGCTTGTAT
AATGTAACCATTTGGGGTCCCGCTTTTACTTGGACTANTGTAACTCCTTCGTGCCAT
AAACTGAAACAGCCATGCTGCTATCTT
BSK-1D8-B3 - reverse
CTGAAAACAAGTTTTATTTAAATAAGGGTTTAAATACATTACACATAACATTAAAAC
TGAAGGGGAAAAAAAAACCAAAAACCAGTTTGTTACTTCACATGCCATTGGGCAGCT
GCTGCTATTAAGTTGCAAGCTCTACAGCTAGCTACATGACTGATGGATCAGTTTGAG
ATTTGTTCCCTTGTCAAAAGTTTAACTCTGATAGAAGGTTGGCCTCACATTCTGATG
TTTGGACATCCCTTAGCTAGGATATGTCTGGTCGAACAGACCTTTGTGGCAAGCCAG
ATGTCCTATCACCTCGCTAGCGGTAAGAGGGCCTCTTTGAGCTCTGTCCACCTAGTC
AGGTTGGAGACACCAGGGGATCTACCACCAAAAGCTCCCTTNTAGTAGTACAGCTGG
GCTTCTGCCTTACCCCATCCTCTCCTTTTAAAATTCACCGANGACTGTTCANGTGGT
AACATTCTTTANGGTANGGAACTCTTGNAAANGGAGAGCTGAGGAGGTTCCCGCCAG
BSK-1D9-A11 - forward
GTGGAGTCTGACTTAGCAAGCCTCGGGTGGGTTTGAGGGTCAAATTTCTACCAGGCT
TATATCCCTGGTGATGCTGCAGAATTCCAGGACCACACTTGGAGGTTTAAGGCCTTC
CACAAGTTACTTATCCCATATGGTGGGTCTATGGAAAGGTGTTTCCCAGTCCTCTTT
ACACCACCGGATCAGTGGTCTTTCAACAGATCCTAAAGGGATGGTGAGAGGGAAACT
GGAGAAAAGTATCAGATTTAGAGGCCACTGAAGAACCCATATTAAAATGCCTTTAAG
TATGGGCTCTTCATTCATATACTAAATATGAACTATGTGCCAGGCATTATTTCATAT
GACAGAATACAAACAAATAANATAGTGATGCTGGTCAGGCTTGGTGGCTCATGCCTG
TATTCCCTAAACTTTGGGAGCCTAAGGNGANAACTCCTTGAACTCCTAAGGCCNGGA
ATTCAAGACCACCTGGATAACATANCAAGACCCCTTCTNTCCNAAAACCAAACCCAA
CCAANCANNANTGAAANGGG
BSK-1D9-B1 - forward
CTTGGGATTGGTGGCGACGACTCCTGGAGCCCGTCAGTATCGGCGGAAAACCGGCCAGA
GGCTGTTCACCTTGGAGACCTGCTGCGGATATGGGTACGGCCCGGCGCGAGATAAACACC
CTCTCCCCCGGATTTTCAGGGGCCAGCGAGAGCTCACCGGACGCCGCCGGAACCGCGAC
GCTTTCCAAGACACGGGCCCCTGTCTCGGGGCGAACCCATTCCAGGGCGCCCTGCCCTTC
ACAAAGAAAAGAGAACTCTCCCCGGGGCTCCCGCCGGCTTCTCCGGGATCGGTCGCGTT
ACCGCACTGGACGCCTCGCGGCGCCCATCTCCGCCACTCCGGATTCGGGGATCTGAACCC
GACTCCCTTTCGATCGGCCGAGGGCAACGGAGGCCATCGCCCGTCCCTTCGGAACGGCGC
TCGCCCATCTCTTAGGAGCGACTGACCCATGTTCAACTGCTGGTTCACATGGAACCCTTCT
TCACTTCGGCCTTCAAAAGTTTTCGTTTGAATATTTGCTACTACCACCAAGATCTGNACCT
GCGGGGGTTCCACCCGGGCCCGCGCCCTANGCTTTAAAGGTTNACCGNAACGGGCCTTCT
ACTTNTCGCGGNGTAACGTCCCCNGGGCTTCCGGGGCGGGGAGCGCGGAATTTCAACTG
ACGCCGGTCGCACCATTACCAANTGGTCTGGNGGCAAAAATAANATAACCGGGCAGGCC
TGTNAACCCAATTCAACAAATGGGGGCCGTNCTATGGATCCCAACTCGGNCCAACTTGA
NCATANTTGNGNTTTTTANGGANCNAAAANCTTGGNGAANNANGGNAAACTTTTCTTGN
GGAATGGTNTCGTTCAATNCCCAANAACAACCGAACTAAAGNGAAACCGG
BSK-1E2-C24 - forward
GCCGAGGATGGCCGTCATGGCGCCCCGAAGCCTCGTCCTGCTACTCTCGGGGGCCCTGGC
CCTGACCCAGACCTGGGCAGGCTCCCACTCCATGAGGTATTTCTCCACATCCGTGTCCCG
GCCCGGCCGCGGGGAGCCCCGCTTCATCGCCGTGGGCTACGTGGACGACACGCAGTTCG
TGTGGTTCGACAGCGACGCCGCGAGCCAGAGGATGGAGCCGCGGGCGCCGTGGATAGAG
CAGGAGGGGCCGGAGTATTGGGACGAGGAGACAGGGAAAGTGAAGGCCCACTCACAGA
CTGACCGAGAGAACCTGCGGATCGCGCTCCGCTACTACAACCAGAGCGAGGCCGGTTCT
CACACCCTCCAGATGACGTTTGGCTGCGACGTGGGGTCGGACGGGCGCTTCCTCCGCGGG
TACCACCAGTACCCTACGACGGCAAGGATTTACATCGCCTGAAAGAAGACCTGCCTCTTGG
ACCGGGGNGGACATGGCGGTTAANATAACAAACGCAAGTGGGANGCGGGCCATGNGGG
BSK-1E2-C24 - reverse
GATGATTGGGGAGGGAGCACAGGTCAGCGTGGGAAGAGGGTCATGGTGGACATGGGGG
TGGGGTGGTGCTAANACAAGGTANAGTANGANATACTTTTCTTACCTNTTTATGCTGA
BSK-1H5-A1 - forward
CTTCAACAAGATAGAAATCAATAACAAGCTGGAATTTGAGTCTGCCCAGTTAACCCCAACTG
GTACATCAGCACCTCTCAAGCAGAAAACATGCCCGTCTTCCTGGGAGGGACCAAAGGCG
GCCAGGATATAACTGACTTCACCATGCAATTTTGTGTCTTCCTAAAGAGAGCTGTACCCAG
AGAGTCCTGTGCTGAATGTGGACTCAATCCCTAGGGCTGGCAGAAAGGGAACAGAAAGG
TTTTTTGAGTACGGCTATAGCCTGGACTTTCCTGTTGTCTACACCAATGCCCAACTGCCTGC
CTTAGGGTAGTGCTAAGAGGATCTCCTGTCCATCAGCCAGGACAGTCAGCTCTCTCCTTT
CAGGGCCAATCCCCAGCCCTTTTGTTGAGCCAGGCCTCTCTCACCTCTCCTACTCACTTAA
AGCCCGCCTGACAGAAACCACGGCCACATTTGGTTCTAAGAAACCCTCTTGTCATTCGCT
CCCACATTCTGATGAGCAACCCGTTTCCTATTAATTAATTAATTTGGTNGGAAGGAAAAA
CATTGGCTAATTTATTCAAAGGGGGG
BSK-1H5-A1 - reverse
CAGGGAAGTTTATTTCAAAACCATTGAACAGTATGATATTTGCTCATTTATAAATAAACCC
ATTTAAATAATCTGAGCTTATATATTTTCAGTCTTAATTAAAGGACTTGATTTAAAGAGAG
CACACCAGTCCAAATTGAATTGATTCCATAGCTATTAAAAACTAGGCTCTTTTACAGACA
CTGCTACTTCTTGCCCCCTTTGAATAAATTAGACCAATGAATAAAACAAACAAACAAATA
AATAAATAAATAGGGAAGCGGTTGCTCATCANAATGTGGGAGCGAATGACAGAGGGAA
CTTANAACCAAATGTGGCCCGTGGTTTCTGTCAGGCGGCTTTAAGTGAGTAGGAAAGGTG
AGAGAGGCCTGCTCAACAAAAGGGCTGGGGATTGGCCCTGAAAGGANAAAGCTGACTGC
CTGCTGATGGACAGGAAATCCTTTACACTACCCTAAGGCAGGCAGTAGGCATTGGGTAA
ACACAGGAAGACG
BSK-1L2-B15 - forward
CGTTCATGGGGAATAATTGCAATCCCCGATCCCCATCACGAATGGGGTTCAACGGGTTAC
CCGCGCCTGCCGGCGTAGGGTAGGCACACGCTGAGCCAGTCAGTGTAGCGCGCGTGCAG
CCCCGGACATCTAAGGGCATCACAGACCTGTTATTGCTCAATCTCGGGTGGCTGAACGCC
ACTTGTCCCTCTAAGAAGTTGGGGGACGCCGACCGCTCGGGGGTCGCGTAACTAGTTAGC
ATGCCAGAGTCTCGTTCGTTATCGGAATTAACCAGACAAATCGCTCCACCAACTAAGAAC
GGCCATGCACCACCACCCACGGAATCGAGAAAGAGCTATCAATCTGTCAATCCTGTCCGT
GTCCGGGCCGGGTGAAGGCAGTGAGCTGAGATTGCGCCACTGCACTCCAGCCTGGGCGA
CAGANCGAGACCCCATCTCAAAAAAAAAGGGGGGGGGTGGACAGGGGGCAAGTGGAGT
CTGGCTTGCCAAAACTACTTGTTGATGGNGGGGAAAAAAAAATGGGTGNCTTCCTCCTTG
GCACTGGGAAAGGTTTTGGTTCTTTTTTTCAT
BSK-1L2-B15 - reverse
GAAAGTTTAGAAACTTTAAAACAATAATAATGACGGTGATAGTGATAATAATTGCTAAT
GCTTCAGATCACATATGTGTTAGGCGCTGTTTTTTGTTGTTGTTGTTATTGTTGAGACAG
TCTCACTCTGTTGGCCAGGCTGGAGTGCAGTGGTGCTTTGCCTCCTGGGTTCAAGGGAAA
CTCCTGCCTCAGCCTCCTGAGTAGCTGGGATTACAGGCATGCGCCACCACGTCGGGCTAA
TTTTTGCATTTTTAGTGGAGACGGGGTTTCATCATGTTGGCCAGGCTGGTCTCGAACTCAC
GACGTCAAGTGATCCACCTGCCTCGGCCTCCCAAAGTGTTGGGATTACAGGCGTGAGCCA
CCATGCCCAGCCAGCACTGTCTTAAATGCTTTACATATATTATCTCATTTAATCCTCAAAA
TACCTTACAATATAGATACTACTATTATTTCCATTTATATTAATGGCANCTCTGAGGCTCA
AACGATGAACTACTTGCTGGGTTACATGA
BSK-1M13-B2 - forward
CCCAAAAATTACCCAAAGAAGAAGATGGAAAAGCGATTTGTCTTCAACAAGATAGAAAT
CAATAACAAGCTGGAATTFTGAGTCTGCCCAGTTCCCCAACTGGTACATCAGCACCTCTCA
AGCAGAAAACATGCCCGTCTTCCTGGGAGGGACCAAAGGCGGCCAGGATATAACTGACT
TCACCATGCAATTTGTGTCTTCCTAAAGAGAGCTGTACCCAGAGAGTCCTGTGCTGAATG
TGGACTCAATCCCTAGGGCTGGCAGAAAGGGAACAGAAAGGTTTTTAAGAGTACGGCTATA
GCCTGGACTTTCCTGTTGTCCACACCAATGCCCAACTGCCTGCCAAAGGGGTAGTGCTAA
GAGGATCTCCTGTCCATCAGCCAGGACAGTCAGCTCTCTCCTTTCAGGGCCAATCCCCAG
CCCTTTTGTTGAGCCAGGCCTCTCTCACCTCTCCTACTCACTTAAAGCCCGCTGACAGAAA
CCACGGCCACATTTGGTTCTAAGAAACCCTCTGTCAAACGCTCCCACATTCTGATGAGCA
ACCGTTCCCTATTTAATTATTTATTGGTNGTTGG
BSK-1M13-B2 - reverse
GAGNGAAGTTTATTTCAAAACCATTGAACAGTATGATATTTGCTCATTTATAAATATTCC
CATTTAAATAATCTGAGCTTATATATTTTCAGTCTTAATTAAAGGACTTGATTTAAAGAGA
GCACACCAGTCCAAATTGAATTGATTCCATAGCTATTAAAAACTAGGCTCAATACAGAC
ACTGCTAGTTCTTGCCCCCTTTGAATAAATTAGACCAATGAATAAAACAAACAAACAAAT
AAATAAATAAATAGGGAAGCGGTTGCTCATCANAATGTGGGAGCGAATGACANAGGGTT
TCAATTANAACCAAATGTGGCCCGTGGTTTCTGTCAGGCGGCTTTAAGTGANTAGGAAAGGT
GAAAGAGGCCTGCTCAACAAAAGGGCTGGGGATTGCCCTGAAAGGANANAGCTGACTG
GCCTGCTGATGGACAGGAAACCTTACACTACCCTAAGCNGCANTGGCCAAAGGTGNGG
ACACAGGAAAG
BSK-2C5-B3 - forward
CGGAATCCCGAAAAAATCGCACATACATTCCTAAAGTTTATATAAACAAGGAAAGAAAA
GAAGGAAAAGAAAGCCGAAGAGCTTTGATTTTTGAAAGGAAAGTTTTGAGTACCTGACT
GAACACCCAACTTGAACTCCGTTCAACTTGCCTGAACAATCCAACAGCTGACTGCTGGAA
CAAGGAAAGGTAGGCACAGCAACAGGCAGGCTAATGGGATTTCTGAAACAGATACAGTA
CCCTGCCATGTTCTTTCACCTGCAAGGGGGAGGGGTGTGTGTGTGTGTGTCTGTGTATAC
ACCGTAAGTGCTAAAGTGCCTGAAAGGCACCTGGGAACAGTACTTAACTGACACCAAGG
TAAGAGACTGTCAGTTCAGCTGANCTAAGGGGAAAACGGCAATTAGGTGTGCCACTGAG
ATTGCTATGTGTAAGGTCCTCCCCTTCTCCTGCAACTTTTCTTCTATTTCTTTTTCCTTTCA
CATGAAAGCCCCTATGTCCCACACCAAATGTCTTAACTGAAGGAATTCCCCTTTTCTTAA
CAAGGNAGTTTGAAAACGGAGGG
BSK-2C5-B3 - reverse
GATTCCAACCTTCACAGATAACTGAGTCTTGATTTGACTTCAAGACTTCAGTGGAGGAAG
TAACTACAAATGTGGTAGAAATAGCTAGATAACTAGAAGTGGTGGAGCCTGAAGATCTG
ACTGAATTGCTGCAGTCTCATGATTAAACTTGAACAGATGAGGATTTGCTTCATATGGGT
GGATACAGAAAGTGGTTTCTTGAGATGAAATCTACTGCTGGCAGAGATGCTGTGAACATC
GTTGAAATGACAACAAAGGACTTCGAATATCAGTAAAATCAGTTGATAAAACCAAAGCA
GGGTTTGAGAGGATGCACTCCCAATTTTGAAAGAAGTTCTTGTGTGGGTGAACGCTATCA
TACCAAACAGCATCGCAAGCTACAGATAAATCTTTCGTGATAGAGTCAATTGACGTGACA
AACTTCATTGGTGGCATTTTAAGGCATTGCCACAGTCACCCCAAAACCCGCAGCAGCCAT
CAACAACNGGCAAGACCCTNCACAACAAAAAGATGA
BSK-2D9-A10 - forward
GTCGTTTTCCAGTTTTTCTTTATTACTTATTTCATTCACCATAATTCCAAATTTTAATGACC
ATATCTTTCCTAAAATATCTACATAAAAATCTTGATTATTTAAGAGTAAAAAGAAGGTTTC
TCCTCTAGCTACTTCTGACCTCTTCAATAAATTGTGCCTGATGCTGCCTCCTTTCCTTCCAA
CCACTCACATTAGAATCCTTTTAGTCAAAGTAGTCTGAGGCTGCAGTTGTTTGCAGGATG
TGATCATCCTCAATACCATATTATTTCAGAGTAGCTTAAGTCACCATTCCTAGGCAATTTC
ATAGTAAAAAAATTATTCTAGGAATTCCTGGACCTATAGATATTTCCAAGATCATTACAA
AAATACTTCTTTTTAAATAAAAAAAAATTGCTAATGNACCATGCTGGGAAATTTTTATTT
AAAAAATAGAACTAAACTCTTGAGCTTCAATAATGCTGGCAGATAGATTCTCANGGCCAA
CTACTGGCCTCAAGGAAATGATGGCNCCCCTCAGTTTGGGAAAGG
BSK-2D9-A10 - reverse
TTTGTATTATAGGAACCTATTTTGAAGCTCTTAGAGCTGAGAGTTAAGTGGTCTTTTAATG
GAACTGCTAAGACAAGGTAGAGTAGGAGATACTTTTCTTCCCTCTTTATGCTGAAGTGTT
TTAGTGTTTCTGTCTGTGACTAGGCAGTAACTTTGAAAGGGATAAGATAGGGTTAATAAC
ATATCTACTAAAACTTGGAAAATATACTATATTTTCTGAGATAAAAATCTTTGGATTGAA
AATTACTTTCTGGTGGAATATGGCAAACTGACATTCATTCAATGTAAGACTTTTTTTCCCT
CACTTTTTGTGTTTTCATCTGTAGTTTTTTTTTTTCTTTTTTACCTGTGGTACCATTTTTAAG
GTGAATCAGGCCAGTTTCANCAAAAAATGGNTGTACTGTTCATCACTTCAGTAGAAGGTA
GGATGACTTCGATGANGGTGNGCTCAGTAACTTCTCTGGTGCTGAATTAGGGCCTGGGAC
AAANAAGGATCCCATCTTACAAATAATGACAANGGAGACTACNGAATCCGGGAG
BSK-1G13-C15 - forward
CGGAATCCGTTTTAAGATGGAGTGTCATTCTGTCACCCAGGTTGGAGTGCAGTGGCGTGA
TCATGGCTCACAGCAACCTCTGCCTCCCAGGTTCAAGCAATTCTCCTGCCTCAGCCTCCTG
AGTAGCTGGGATTACAGGTGCCCGCCAGCACGCCCAGCTAATTTTTGTATTTTTTAGTAGA
GACAGGGTTTCACCATGTTGGCCAGGCTGGTCTCGAACTTTTGACCTCAGGCGATCCACC
TGTCTCCGGAATTCGGGTTACGGCAGCACTTTTATTTTTCCTTACACAATGACGTGTTGCT
GGGGCCTAATGTTCTCACATAACAGTAGAAAACCAAAATTTGTTGTCATCTCTTCAAAGA
ATCGAGAATTGCGTACAAAAAAAACCTTACATAATTAAGAATGAATACATTTACAGGC
GTAAATGCAAACCGCTTCCAACTCAAAGCAAGTAACAGCCCACGGTGTTCTGGCCAAAG
ACATCACTAAGAAAGGAAACTGGGTCCTACGGTTGGACTTTNCACCCTGACAGACCCGC
AAGACAAAACAACTGGTTCTTGC
BSK-1G13-C15 - reverse
CGGAGGAGCACCCAGTGCTGCTGACCGAGGCCCCCCTGAACCCCAAGGCCAACAGAGAG
AAGATGACTCAGATTATGTTAAGAGACCTTCAACACCCCGGCCATGTACGTGGCCATCCAG
GCCGTGCTGTCCCTCTACGCCTCTGGGCGCACCACTGGCATTGTCATGGACTCTGGAGAC
GGGGTCACCCACACGGTGCCCATCTACGAGGGCTACGCCCTCCCCCACGCCATCCTGCGT
CTGGACCTGGCTGGCCGGGACCTGACCGACTACCTCATGAAGATCCTCACTGAGCGAGG
CTACAGCTTCACCACCACGGCCGAGCGGGAAATCGTGCGCGACATCAAGGAGAAGCTGT
GCTACGTCGCCCTGGACTTCGAGCAGGAGATGGCCACCGCCGCATCTCCTCTTCTCTGGA
GAAAACTACGACTGCCGATGGCANGTCATACCATTGGCATGAGCGGTTCCCGGGTCCGG
AGGCGCTGTNCANCCTTCTTCTGGGNATGG
BSK-2G14-B4 - forward
CTTTACTAAAAATACAAAAATTAGCCAGGCATGGTGGCAGGTGCCTGTAATCCCAGCTAT
TCGGGAGGCTGAGGCAGGAGAATCACTTGAACCCAGGAAGTGGAGGTTGCAGTGAGCCG
AGATCGTACCACTGCACTCCACCCAGGGTGACAGAGTGAGACTCCGTTGAAAAAAAGAG
AAAAAAAAATTAATACAAAGATATTAAAATTAAAAAGGAAAAATATCCCCAGAACCCCA
TCACTTAAACAACAAATCAAATTTTTATTTTTCTCTTCCCATCCTACAAGGCAACATAACT
CTGACCTGCTTAGAATCCCCGTGTCAGGCCACTTTCCTATTCTGTTTCTTCCCACTCCTCA
CCGTGCCCACACACCTTCCTTGGGGGTGAACGCGTGCGGACGGTAGACGGCCCCTCATCC
CCCGACTGCCTGCCCGGGTGGAACTG
BSK-2G14-B4 - reverse
CAGTTCCACCCGGGCAGGCAGTCGGGGGATGAGGGGCCGTCTAGCGTCCGCACGCGTTC
ACCCCCAAGGAAGGTGTGTGGGCACGGTGAGGAGTGGGAAGAAACAGAATAGGAAAGT
GGCCTGACACGGGGATTCTAAGCAGGTCAGAGTTATGTTGCCTTGTAGGATGGGAAGAG
AAAAATAAAAATTTGATTTGTTTGTTTAAGTGATGGGGTTCTGGGGATATTTTTCCTTTTTA
ATTTTAATATCTTTGTATTAATTTTTTTTTCTCTTTTTTTCAACGGAGTCTCACTCTGTCACC
CTGGGTGGAGTGCAAGTGGTACGATCTCGGNTCACTGNAACCTNCACTTCCTGGGTTCAA
GTGATTCTCCTGCCTCACCTCCCGAATAGCTGGGATTACAGGCACCTGCCACCATGCCTG
GCTAATTTTTGNATTTTTAGTAAAG
BSK-2G9-A1 - forward
CTTCAGCGAAGTTTATTTCAAAACCATTGAACAGTATGATATTTGCTCATTTATAAATATT
CCCATTTAAATAATCTGAGCTTATATATTTTCAGTCTTAATTAAAGGACTTGATTTAAAGA
GAGCACACCAGTCCAAATTTGAATTGATTCCATAGCTATTAAAAACTAGGCTCTTTTACAG
ACACTGCTACTTCTTGCCCCCTTTGAATAAATTAGACCAATGAATAAAACAAACAANCAA
ATAAATAAATAAATAGGGAAGCGGTTGCTCATCANAATGTGGGAGCGAATGACAGAGGG
TTTCTTANAACCAAATGTGGCCGTGGTTTCTGTCAGGCGGCTTTAAGTGATAGGAAAGGT
GAAAAAGGCCTGGCTCAACAAAAGGGCTGGGGATTGGCCCTGAAAGGANAAACTGACT
GCCTGCTGATGGACAGGAAACCTNTTACCCTCCTANGCNGCNNTTGGGCTTGGGGGNAA
CACNGGANAGCCNGGNThFACCCGACCCNAAAG
BSK-2G9-A1 - reverse
CCCATCATCAATATTTATTGAGCATTTACAGTGTACTAGGCACAATAGAACATACAGAAA
ACATTGTCCCTGCTCTTGAGGAGCTTACATTCTAAAAGAAAAAATACACCTTTTTTAAAA
TGGCATTTTTGTTTGGTGTTTTCTGCAAAGTACTGAGGAAATATTTTGTAAAGTGAGCTTT
GGCCTTGGGCCTCAAGGAAAAGAATCTGTACCTGTCCTGCGTGTTGAAAGATGATAAGCC
CACTCTACAGTTGGAGAGTGTAGATCCCAAAAATTCCCAAAGAAGAAGATGGAAAAGC
GATTTGTCTTCAACAAGATAGAAATCAATAACAAGCTGGAATTTGAGTCTGCCCAGTTCC
CCAACTGGTACATCAGCACCTCTGAAGCANAAAACATGCCCGTCTCCCTGGGAGGGACC
AAAGGCGGCCAGGATATAACTGACTTCACCATGCAATTGNGTCTTCTAAAGAAGAGCT
GACCCAAAAAGTCCTGNGCTGAATGNGGACTCAATCCCTAGGCTGGGCANAAAGGG
BSK-2G9-B1 - forward
CCCTTAGAGCCAATCCTTATCCCGAAGTTACGGATCCGGCTTGCCGACTTCCCTTACCTAC
ATTGTTCCAACATGCCAGAGGCTGTTCACCTTGGAGACCTGCTGCGGATATGGGTACGGC
CCGGCGCGAGATTTACACCCTCTCCCCCGGATTTTCAAGGGCCAGCGAGAGCTCACCGGA
CGCCGCCGGAACCGCGACGCTTTCCAAGGCACGGGCCCCTCTCTCGGGGCGAACCCATTC
CAGGGCGCCCTGCCCTTCACAAAGAAAAGAGAACTCTCCCCGGGGCTCCCGCCGGCTTCT
CCGGGATCGGTCGCGTTACCGCACTGGACGCCTCGCGGCGCCCATCTCCGCCACTCCGGA
TTCGGGGATCTGAACCCGACTCCCTTTCATCGGCCGAGGGCAACGGAGGCCATCG
BSK-2G9-B1 - reverse
GCGATGGCCTCCGTTGCCCTCGGCCGATCGAAAGGGAGTCGGGTTCAGATCCCCGAATCC
GGAGTGGCGGAGATGGGCGCCGCGAGGCGTCCAGTGCGGTAACGCGACCGATCCCGGAG
AAGCCGGCGGGAGCCCCGGGGAGAGTTCTCTTTTCTTTGTGAAGGGCAGGGCGCCCTGG
AATGGGTTCGCCCCGAGAGAGGGGCCCGTGCCTTGGAAAGCGTNCGCGGTTCCGGCGGC
GTCCGGTGAGCTCTCGCTGGCCCTTGAAAATCCGGGGGAGAGGGTGTAAATCTCGCGCC
GGGCCGTACCCATATCCGCACAGGTCTCAAGGTGAACAGCCTTGGCATGTTGGAACAAT
GTANGTAAGGGAAG
BSK-2G9-C3 - forward
CAAACCCACTCCACCTTACTACCAGACAACCTTAGCCAAACCATTTACCCAAATAAAGTA
TAGGCGATAGAAATTGAAACCTGGCGCAATAGATATAGTACCGCAAGGGAAAGATGAAA
AATTATAGCCAAGCATAATATAGCAAGGACTAACCCCTATACCTTCTGCATAATGAATTA
ACTAGAAATAACTTTGCAAGGAGAGCCAAAGCTAAGACCCCCGAAACCAGACGAGCTAC
CTAAGAACAGCTAAAAGAGCACACCCGTCTATGTAGCAAAATAGTGGGAAGATTATAG
GTAGAGGCGACAAACCTACCGAGCCTGGTGATAGCTGGTTGTCCAAGATAGAATCAAAG
TTCAACTTTAAATTTGCCCACAGAACCCTCTAAATCCCCTTGTAAATTTAACTGTTAGTCC
AAAGAGGAACAGCCCTTTGGACACTAGGAAAAAACCTTGTAGAGAGAGTAAAAAATTTA
ACACCCATAGTAGGCCTAAAAGCCGGAATTNCAGCTTGAGCGCCGGTCGTTCCAAAACCA
GNCGGTCTGGGGGTCAAAAATATAATAACG
BSK-2G9-C3 - reverse
GCTTTTAGGCCTACTATGGGTGTTAAATTTTTTACTCTCTCTACAAGGAATTTTTTCCTAGTGT
CCAAAGGGCTGTTCCTCTTGGACTAACAGTTAAATTTACAAGGGGATTTAGAGGGTTCT
GTGGGCAAATTTAAAGTTGAACTAAGATTCTATCTTGGACAACCAGCTATCACCAGGCTC
GGTAGGTTTGTCGCCTCTACCTATAAATCTTCCCACTATTTTGCTACATAGACGGGTGTGC
TCTTTTAGCTGTTCTTAGGTAGCTCGTCTGGTTTCGGGGGTCTTAGCTTTGGCTCTCCTTGC
AAAGTTATTTCTAGTTAATTCATTATGCAGAAGGTATAGGGGTTAGTCCTTGCTATATTAT
GCTTGGCTATAATTTTTCATCTTTCCCTTGCGGNACTATATCTATTGCGCCAGGTTTCAAT
TTCTATCGCTATACTTTATTTGGGTAAATGGTTGGCTAANGGTGCTGGTATAAGNNCAGN
GGGTT
BSK-2H10-A4 - forward
TTGAACGCTTTCTTAATTGGTGGCTGCTTTTAGGCCTACTATGGGTGTTAAATTTTTTTACT
CTCTCTACAAGGTTTTTTCCTAGTGTCCAAAGAGCTGTTCCTCTTTGGACTAACAGTTAAA
TTTACAAGGGGATTTAGAGGGTTCTGTGGGCAAATTTAAAGTTGAACTAAGATTCTATCT
TGGACAACCAGCTATCACCAGGCTCGGTAGGTTTGTCGCCTCTACCTATAAATCTTCCCA
CTATTTTGCTACATAGACGGGGTGTGCTCTTTTAGCTGTTCTTAGGTAGCTCGTCTGGTTT
CGGGGGTCTTAGCTTTGGCTCTCCTTGCAAAGTTATTTCTAGTTAATTCATTATGCAGAAG
GATAGGGGTTAAGTCCTTGCTATATTATGCTTGGGTATAATTTTTCATCTTTCCCTTGCGG
TACTATATCTATTGCGCCAGGTTTCAATTTCTATCGCCTATACTTTATTTGGGTAAATGGN
TTGCTAAAGGTGNCTGGTAATAAGGTGGAATGGGTTTGCGGA
BSK-2H10-A4 - reverse
CANNCCCACTNCANCTTACTACCNGACATCCTTANCCAAACCATTTACCCAAATANAGTA
TAGGCGATAGAAATTGAAACCTGGCGCAATAGATATANTACCGCAAGGGAAAGATGAAA
AATTATAACCAAGCATAATATAGCAAGGACTAACCCCTATACCTTCTGCATAATGAATTA
ACTAGAAATAACTTTGCAAGGAGAGCCAAAGCTAAGACCCCCGAAACCAGACGAGCTAC
CTAAGAACAGCTAAAAGAGCACACCCGTCTATGTAGCAAAATAGTGGGAAGATTTATAG
GTAGAGGCGACAAACCTACCGAGCCTGGTGATAGCTGGTTGTCCAAGATAGAATCTTAG
TTCAACTTTAAATTTGCCCACAGAACCCTCTAAATCCCCTTGTAAATTTAACTGTTAGTCC
AAAGAGGAACAGCTCTTTGGACACTAGGAAAAAACCTTGTAGAGAGAGTAAAAAATTTA
ACACCCATAGTAGGCCTAAAAGCAGCCCCAATTAAGAAAGCGTCAACGGAATTNCAGCT
GAGCGCCGGTCG
BSK-4-4 - forward
GCTGGAAATACAGCAATGAATAGGTCTCTAGTCTCCTGGAACATCAAATGATGTTTATCC
AAAAGTATAAATAGTTACCATTTTTATTGTCTTCTTAATAAATTGAATAAAATAATGTCT
TTGCTGCCAGTAACATGGATGGAACTGGAAGTCACTATTTTAAGTGGAATTAAAGAAAA
AGAAAGTCAAATACCATAGGTTCTCACTTATAAGTGGGAGCTAAATAATGTATACACATA
GACGTAGAGTGTGAAATAATAGATATCGGAGACTCAGAGAATTGTTTTGTTTGAGGAGG
CTGAAGATAGGACCCCAATCCCTTCTAGCTTGTAGGGTTTCTGCTGAGAAATCTGTGGTT
AATCTAAGTTTCCCTTTATAGGTTACCTGGTGCTTTTGCTCACAGCTCTTAAGATTCTTTN
CTTCGCTTAACTTTGGCTAACCTGGTGACAATATGCCTANGCGATGATCNTTTTGNGATA
AATTTTTCAAGTGGTCTTTGTGCCTAAGNCTCTAGCAGACTTGGGGAAGTTTTCCTTGATA
TTTCCCCAAATATGGTTTTCAAGCTTTANAATCTCTTCTTTCTCAGGAACCCCGATATTCT
TAAGGTTGNCCTTGAGCTNATCCCAANTTTTTTGAGGTTTGTNAAATGGGCTAAAANAAT
TCTTTGCNTTTNANGNATGGGNTCANTTTNAAAACCTTGNTTTTAANCCNCGAAAT
BSK-17 - forward
CTGTGTTAGAAAAAATCATAAAACATAACAGAATCTACACATCATGGTCCACCAGAGGA
TTCACAGATGGAAATGAATTTTAATATTGTTACTTTTGAAGTCCCAAATACTTTAAGATTT
ACAATAAAAAACATTCTGACAGAGTCCATGATGAATTATTTCCAGTCTTTCACCAGACTG
CTTAAGCTCACCTATAAACTACGAAATGTATAAATAAATAATTACAGCCAAAGCAGGTA
ACAAAGTGTCTAACCTATATTTCCACAGGTGCATACCATGGCTACGAATAAACTATCCAAT
CTAACCACAGAAGCTGAGCATTTGGTTTGGGGTTAATCCACATCACATGACTCACCATTG
AGAAAGCGGCTCTCACCATGCTTAATGGGCACAGCACCTCTGCAAACAAATCCTTCCCTG
GCTAATCATTCCCTCTGAGAGGTTTCCTCAGTAAAGAGATTAGAACTACCTCTTGCATTTC
CAACTTTTAAAAAATTGCCTTTTTGGAAATCTACCACCACCAACTAATTCTTGACAGACTT
GTAGAGAATGACCCTCAAAGAAATATCATTCGAGACACATATTCAAGCAGACTGGNCAT
GGTGGCTCATGCCTGCAGTCCCAGCAGTTTGGGAAGCTGAAGTGAACTGATGCTTGAATN
CAGGAGTCTTGAGAACAGCCTGGGTAACATGGNAAAACCGGGTCCTACAAAAAAATTCC
NAAAATTACCCNGGTNTGTTGGNGCACAATGNGGGCCCAACTTTNCCNAAAGAAAAAAG
TTTGGCTTCAGGAAGGCAAGGGTCNCNNANCCCTGAATGGCCCCTTCCTTTCAACCGGGGN
AAAAANGGGNAACCTTTTTGGNAAGGGAAGGGAAAGGGAAAGGGAGGGCCTTTTNNNT
TTAAAAAAGGGANNTTAAAAGGNGGCCCNAAAACNTTTTTAAAGGGCAACCTTTTTNC
TTTTTTGGGAAAATTGGGGNAAAT
BSK-23 - forward
AGGGGATGCTCTCGGTGTCTGAGCTGTTGTTGACAGTGGCTGGGCCACTGCATTCCCCTC
TGGGCACCTCATTCCCAGAGGCATGTAAGGCTTCAGCCTCCTCCACCATCTCCTCCTCATT
TCCGCTCACGCCCGACGCCTCCATCTCCTCATCCTCCACCACGGGCGGGAATGCAGCCTC
CTCGCTGGCCGCCGCCGGCGCTTTCTTCTTCTTCCTCCGCGCGTTCCTCTCCAACTCCATCT
TCAGCTTGTGCTGCTGCAAGATCTCATCGAGGTTCTGCCTCTTCTTGTAGTTGAAGTAGAA
GTTCTTACACTGCGACACAGTCTTGGAGCCCACCATCCGGGCGATGGCCGACCAAGAAGC
GGCCGTGTTCCAGGAGACCTTTCTTGGCTGNTTCCATTTCTTCTTCTGTCCAGCGAGAACT
CTCATTCACTTCANGGAGGCCAGCTCGGCGCTCTGCTGGGGGGTGATGGCCTCCTCGAAG
TGGCCTCATTAGCCCTTGAACCGGGTGATGCGGGCCTTTGGNCTTCCCTGGCTTGAAGCA
ANTTTGCGGCCTTTGGAGGCCACAGCTTCCTTTTGGNGGTTGGNCCTCCCCTGAGGGGNC
GCTGGCTTNTCCTTGAGGANGGCTTCCTTGGGGTNTTCACCTCGGGTTTTCCCTCTTTTCG
GGTTCNTTTTCCGGAATCCCCNAATTGACGGTTCAGAATTTNGCCCAATCCA

These genes or tools that make use of these genes are preferably used to characterize the molecular processes, in particular, in the monocyte/macrophage system, for the diseases mentioned.

As can be seen, the genes in Table 1 can be divided into various function groups:
cytokines, soluble factors, messenger substances and ligands that exert a controlling influence on the inflammatory process;
receptors, ion channels and associated proteins that activate a signal chain as soon as they are activated themselves;
kinases, protein kinases and their antagonists that are the switches for activation and forwarding or blocking of the intracellular signal transmission;
signal transduction molecules that are part of the intracellular signal chain and contribute to controlling gene expression or other secondary mechanisms (activation of preformed molecules);
Transcription and translation factors and associated molecules that are involved in regulating specific gene expression and protein synthesis;
Ribosomal and ribonuclear regulator proteins that are involved in protein biosynthesis;
enzymes and enzyme-associated proteins that have a regulatory function for other signal molecules;
proteinases, matrix metalloproteinases, enzymes and their inhibitors that influence decomposition of the tissue matrix and the resulting organ damage;
oncogenes, protooncogenes, differentiation factors and genes from embryonal development that influence cell development; (proliferation, differentiation, dedifferentiation);
apoptosis genes and regulators of the cell cycle and cell death;
acute phase proteins that indicate cell activation and inflammation;
surface membrane molecules that characterize a specific cell type or cell population;
other molecules that are found in increased quantities with the inflammatory disease mentioned but cannot be assigned to any of the above groups;
gene sequences as yet not characterized in any detail that show differing degrees of expression in their own differential gene expression analyses;
control genes that do not show any gene expression between chronic inflammation and controls and are required for standardization, comparability, and quantification of an array analysis.

The genes or gene sequences according to the invention may also be allels, derivatives or splicing variants.

These genes were derived from differential gene expression analyses by gene subtraction from blood and tissue samples of chronic inflammatory diseases and suitable control samples (samples from regular donors or patients that have degenerative diseases).

Advantageously, a gene pool was created by this type of selectioning that is both specific to the inflammation and to the cells of the monocyte/macrophage system. Subtractive gene processes allow the preselection of differentially expressed genes. This includes methods such as differential hybridization or the PCR-based representational difference analysis (RDA) and differential display (DD). Using subtractive methods, the number of genes or their partial sequences to be applied to a chip can be considerably reduced and specified, so these are therefore of greater importance for the chip method when being inflammatory or cell-specific.

Analysis of gene expression in inflammatory diseases is to be focused on the genes and gene groups mentioned above using genetic information (cDNA, its partial sequences or corresponding oligonucleotides, RNA) using an array or fluorescence cytometric method or specific oligonucleotide pairs in a quantitative PCR technique. Preferably, only the genes in the gene groups mentioned above or a partial group thereof are to be used for quantitative DNA/RNA high throughput gene expression analysis. Alternatively, a more comprehensive array (such as the UniGene array) can be used and focused on the genes listed in the bioinformation analysis step.

The molecular tool according to the invention provides an opportunity to characterize a disease in molecular terms and, based on this characterization, 1) determine a molecular classification and stages of a clinically defined inflammatory disease, 2) establish an individual prognosis profile, 3) make proposals for molecular pathogenesis, 4) determine treatment effects and ways of treatment monitoring, and 5) develop new treatment strategies and pharmacological concepts.

In this way, DNA/RNA microarrays and the fluorescence cytometric technique are to be used for diagnosis, forecasting, and treatment monitoring. DNA/RNA microarrays are arrangements of molecular species on a carrier that are used to detect interacting species (such as complementary nucleic acids).

Therefore, the gene sequences used in the methods protection is applied for as tools of inflammatory diseases from claim 1 are not accidental but were subjected to prior selectioning.

Suitable substrate materials (glass, plastic) are used for the array and microarray technique and chemically activated or modified, reactively coated with reactive groups that bind amino linkers, metal compounds or alloys to achieve permanent bonding of the cDNA, partial cDNA sequences, oligonucleotides, and RNA from the gene sections selected or the genes to be examined. Application of the individual cDNAs, RNAs or oligonucleotides is site-specific using suitable printing methods. Alternatively, lithographic methods of synthesis can be applied to synthesize the respective oligonucleotide sequences directly and with site specification on the substrate.

For DNA microarrays, cDNAs coupled via amino linkers and DNA oligomers are preferably applied onto modified and chemically activated glass surfaces of the biochips.

According to the invention, a DNA microarray for diagnostic, prognostic and therapy-monitoring analysis of chronic inflammatory diseases, bacteria-induced inflammations, tumors, arteriosclerosis and sepsis is provided to the surface of which a great number of selective monocyte/macrophage genes, gene sections, and oligomer sequences are bound for detection.

The selective monocyte/macrophage genes, their gene sections or oligomer sequences can be used in an RNA microarray.

The selective genes or their gene sequences preferably are a gene library bound in solid phase that is preferably provided in the form of a cDNA library cloned into phages or plasmides.

The cDNA library at least comprises the genes and gene sections listed below, which are representative of the diseases mentioned above.

The cDNA molecules, their partial sequences or their inherent oligomer sequences may either be monocyte/macrophage genes whose function is known or genes whose function is not known or that have not been known as yet.

The genes whose function is known are important for the analytical aspect of the application and should therefore be considered in addition to the genes with unknown functions or completely unknown genes listed in Appendix 1.

Suitable substrate materials (glass, plastic) are used for the array and microarray techniques and chemically activated or modified, reactively coated with reactive groups that bind amino linkers, metal compounds or alloys to achieve permanent bonding of the cDNA, RNA, or oligonucleotides from the gene sections selected or the genes to be examined. Alternatively, substrates (nylon membranes) are used for coating. Application of the individual cDNAs, RNAs or oligonucleotides is site-specific using suitable printing methods (piezo or pin technique). Alternatively, in-situ methods of oligonucleotide synthesis can be applied to synthesize the respective oligonucleotide sequences directly and with site specification on the substrate.

In a fluorescence cytometric process, individual cDNAs, RNAs, or oligonucleotides from selective gene sections bonded to substrate beads made of plastic or glass are used that have a defined size which can be assigned to each specific nucleotide sequence. These beads can be activated or modified chemically, coated with reactive groups that bind amino linkers or other reactive groups such as metal compounds or alloys to ensure a lasting bond of the cDNAs, RNAs, or oligonucleotides. The cDNAs, RNAs or oligonucleotides are bound by incubating the respective beads of equal size in suitable solutions that contain the respective cDNA, RNA or oligonucleotide and facilitate a firm bond to the beads. The various populations of beads that carry nucleotide sequences, wherein each nucleotide sequence is assigned to a defined bead size or fluorochrome, are mixed at even bead parts so that a defined selection of genes can be examined at the same time in one batch.

When using both array and fluorescence cytometry, the sample to be examined is labeled, either directly with a fluorochrome or with a bridge molecule such as biotin or digoxigenin, for later binding of the fluorochrome with or without signal amplification, preferably using streptavidin or anti-digoxigenin antibodies coupled to streptavidin. When using fluorescence cytometry, detection can be carried out by having fluorescence-specific antibodies detect a defined target antigen on the bead surface using the biotin/streptavidin system, either with or without signal amplification. As an alternative, amplification systems such as metal/noble metal complexes are available for array and fluorescence cytometry. Detection can also be performed with radioactivity using a filter membrane based technique.

The labeled sample is quantitatively hybridized with the beads that carry the array or nucleotide sequence and the result can be read out after final labeling with a fluorochrome using a laser scanner on the array or a FACS analyzer.

In array and fluorescence cytometry, crude data (signal intensity) is analyzed and evaluated biometrically using several commercial software programs or programs such as Microarray Suite (Affymetrix) for the array technique or BD CBA Analysis Software (BD Biosciences).

Several approaches are conceivable and required when it comes to interpreting diagnostic assignment of a disease to a defined molecular classification, estimating a prognosis, monitoring or recommending a treatment, molecular pathogenesis and developing new therapeutic concepts. One option is a purely quantitative comparison of the gene expression of various genes in samples from a diseased person and a control person or just of the diseased person's samples prior to, and at various times during a therapy or the course of the disease, e.g. to interpret pathogenesis or develop new treatment concepts. It is more telling for diagnostic interpretation (division into groups and stages, prognostics, therapy and curative effect monitoring) to use algorithms obtained, for example, by linking the expression values of two or more genes. The algorithm may include adding, subtracting, multiplying, dividing, exponential or logarithmic functions as a single operation or constitute a complex of several operations. In this way, (1) the expression data of all or individual genes of a group or (2) the expression data of all or individual genes from various groups can be correlated by an algorithm.

The tools according to the invention are produced in accordance with the following method:

The selective cell, tissue, and inflammation-specific molecular tools used in the methods are produced by gene subtraction.

cDNA is produced by transcribing the mRNA using reverse transcription. The mRNA was taken from inflammatory human blood monocytes or from the inflamed tissue areas that show high macrophage infiltration in patients suffering from inflammatory diseases, and purified either in the conventional way or, alternatively, by positive or negative selection.

In particular, negative selection is used to prevent activation artefacts that can occur during purification.

The reverse transcribed single cDNA strands are transcribed into double cDNA strands with DNA polymerase. These are cloned in plasmide and/or phage vectors for differential hybridization or, uni- or bidirectionally, via adapter or linker molecules. After transforming the plasmides or wrapping the phage vectors, differential hybridization is carried out using labeled cDNA probes (radioactivity, digoxigenin, biotin, or others).

Alternatively, differential expression analysis can be carried out based on the transcribed cDNA by DD or RDA or using classic gene subtraction methods so that the mRNA of the first cell pool is hybridized with the complementary cDNA of the second cell pool. For using the selective differential cDNAs, the cloned units are preferably amplified with NH₂-labeled standard primers for lambda, T3/T7/SP6/M13 and other vector-specific sequences via PCR, and then purified using the column exclusion method. Then the NH₂-labeled DNA is concentrated by precipitation and subsequent centrifugation.

After coupling the DNA on the modified coupling-capable surface, the probe is hybridized either with cRNA that is directly labeled with dye or with cDNA probes reversely labeled with dye that were taken from patient material (blood or tissue).

In addition to the immobilization method mentioned above using NH₂-cDNAs, there are also methods for spotting or printing (pin or piezo technique) oligonucleotides or for producing DNA arrays that were synthesized in situ. In this way, DNA array wafers of identical quality and sequence can be produced in parallel for these production techniques. For in-situ synthesis, the individual DNA arrays and the spots thereon are actuated on the wafer by a highly integrated printing mask using Microsystems technology. The number of printing pores per DNA array and their geometry allow high integration densities at a size of <3 μm per DNA chip. In a first step of this microscreen printing technique, the mask is applied to the substrate wafer that is positioned by means of alignment marks. Each printing pore is actuated via a channel using a microfluid system for the actual printing process. The respective sample substance (for example, oligonucleotides with a length of 20 to 50 nucleotides) can react with the chemically reactive and modified wafer surface and forms a covalent bond with the array surface. The mask is removed and rinsed after printing. The cycle can be repeated after repositioning (offset by one respective spot unit). The in-situ method can be used to produce replications of low-scale integrated DNA arrays with 400 spots per unit or large-scale integrated arrays with up to 20,000 spots.

The invention is based on the consideration that a selective differential gene spot pattern whose underlying sequences were produced using a highly selectioning method can be used on a microarray, the advantages as compared to known DNA arrays being that these can be produced at reduced cost and that analysis is less expensive and takes a smaller effort.

Blood or tissue-specific RNA molecules from patient samples with chronic inflammatory diseases, bacteria-induced inflammatory diseases, tumors, arteriosclerosis, organ and tissue transplantation and sepsis are bound to the RNA microarrays. Qualitative/quantitative detection of the transcribed quantity of relevant genes is carried out with the species of the selectioned genes, gene sections, or oligomers described in the DNA microarray section. The RNA samples are spotted onto coupling substrates and are composed of total RNA and messenger RNA. The RNA is used as a target for the highly significant expressed genes derived from the DNA microarray that are used as labeled probes for hybridization. It is proposed to couple biotinylized RNA or messenger RNA on glass substrates (slides) coated with streptavidin. After labeling the RNA with biotin derivatives, it is applied by spotting to the glass or plastic slides treated with poly-L-lysine, but preferably coated with streptavidin, and dried. This prevents degradation of the RNA. Alternatively, the RNA can be bonded to reactive substrate materials to achieve a covalent coupling, preferably with UV radiation as a catalyst.

In addition, multiple, simultaneous labeling of various genes, gene units or oligomers with various labeling species such as radioactivity, fluorescein, digoxigenin and enzymatic labelers is preferred.

It is possible to label the probes with different fluorochromes at the same time. Enzymatic or radio labeling are potential alternatives. Labeled household genes (alpha, beta, gamma-actin, GAPDH, etc.) are used for quantification and quality control. Detection is preferably carried out in parallel and simultaneously using up to 50 gene probes per batch at the same time.

In addition to simplifying biometric analysis by coupling RNA species to substrates, this system allows fast diagnoses and provides complex, patient-focused, fast diagnosis, prognosis, and treatment control. The system enables fast performance at high throughputs, in particular, for pharmacological development strategies.

As this application refers to the phenotypic cell assignment, i.e. the monocyte/macrophage system, and as the genes, gene units or oligomers were pre-selectioned, many approaches and methods can be applied that are reflected in simultaneous or separate applications of DNA/RNA microarrays for the diseases mentioned herein. These methods combine commercial usefulness with usefulness in solving scientific problems.

The labeling functions of these molecular tools in the DNA microarray/DNA fluorescence cytometric microarray, RNA microarray or reverse transcription polymerase chain reaction (RT-PCR) allow for generating a disease-specific gene expression pattern for each inflammatory process and estimating the relevance of certain biologically active substances and their derivatives as well as for developing new treatment options using conventional chemical pharmaceuticals, pharmaceuticals based on natural substances, antisense RNA, aptamers, ribozymes, or monoclonal antibodies. In addition, species-specific courses of diseases can be recorded or analyzed for all diseases mentioned herein, and the disease-specific treatment can be adjusted individually depending on inflammatory activity.

1. Tools for diagnostic, prognostic and therapy-monitoring analysis as well as for performing screenings for pharmacologically active substances and substance classes of rheumatoid arthritis, chronic inflammatory diseases, bacteria-induced chronic inflammatory diseases, arteriosclerosis, tumors, organ and tissue transplantations, and sepsis,
characterized in that
sequences of a selection of the selective monocyte-macrophage genes listed in Table 1 and/or Table 2 or sequences of all genes listed in Table 1 and/or Table 2 are bonded to the surface of said tool or that RNA complementary to the genes listed is bonded to the surface of said tool.

2. The tools according to claim 1 wherein said genes, partial gene sequences and oligomer sequences are selectioned/subtracted genes.

3. The tools according to claims 1 or 2, characterized in that, alternatively, allels, derivatives and/or splicing variants of the genes, partial gene sequences or oligomer sequences can be present on the tool surface.

4. The tools according to at least one of claims 1 through 3, characterized in that said tools include gene sequences having at least one partial sequence identity in the protein-coding sections of mRNA.

5. The tools according to claim 4, characterized in that said tools include gene sequences having at least 80% of sequence identity in the protein-coding sections of the corresponding nucleic acid.

6. The tools according to claims 1 through 5,
characterized in that they are based on a high-throughput method of DNA array hybridization,
a high-throughput method using fluorescence cytometry (cytometric bead array)
a high-throughput method of RNA array hybridization, or
a high-throughput method using PCR for (semi-) quantification.

7. The tools according to claims 1 through 6, characterized in that the genes have a covalent bond to the surface.

8. The tools according to claims 1 through 7, characterized in that the surface consists of glass or a synthetic material that is chemically activated or modified.

9. The tools according to claim 8, characterized in that their surface is reactively coated with reactive groups that bind amino linkers, with metal compounds, or alloys.

10. The tools according to claims 8 or 9 wherein the substrate material or surface consists of a nylon membrane.

11. The tools according to any one of claims 1 through 10, characterized in that the cDNA was produced by reverse transcription from total RNA or messenger RNA of human cells of the monocyte/macrophage system.

12. The tools according to any one of claims 1 through 10, characterized in that the genes were applied using cDNA spotting methods, immobilization methods and synthesis methods of oligomers or in the form of complementary RNA.

13. The tools according to any one of claims 1 through 12, characterized in that the detection is performed using cDNA, its sequence portions, or oligo probes.

14. The tools according to claim 10, characterized in that the sequences contain deviations.

15. The tools according to claim 11, characterized in that the deviation of cDNA and oligo probes for detection is up to 20%.

16. The tools according to any one of claims 1 through 15, characterized in that the probes for detection are labeled with a fluorochrome, enzyme, protein, or are radio labeled and permit amplification.

17. The tools according to claims 1 through 16, characterized in that coupled biotin, digoxigenin, (noble) metal chelate and protein (molecules) are used for amplification.

18. The tools according to claim 17, characterized in that streptavidin, (noble) metal chelate or antibodies are used for amplification.

19. The tools according to claim 18, characterized in that detection is performed by means of a fluorochrome, radioactivity, or enzymes.

20. The tools according to any one of claims 1 through 19, wherein the tools are formed by a DNA array.

21. The tools according to any one of claims 1 through 19 wherein the tools are formed by a cytofluorescent DNA array (beads).

22. The tools according to any one of claims 1 through 19, wherein the tools are formed by an RNA array that mirrors a DNA array.

23. The tools according to claims 1 through 22, characterized in that the selective genes, their partial sequences or oligomers are used for detecting solid phase bound total RNA or messenger RNA.

24. The tools according to claims 1 through 23, characterized in that the probes are produced by reverse transcription from messenger RNA of human cells of the monocyte/macrophage system.

25. The tools according to any one of claims 1 through 24, characterized in that RNA from up to 500 tissue and/or blood samples is applied to the array.

26. The tool according to any one of claims 1 through 25, characterized in that the probe is labeled with fluorochromes, biotin, digoxigenin, peroxidase, alkaline phosphatase and radioactivity.

27. The tool according to any one of claims 1 through 26, characterized in that detection of the sequences listed is to be carried out using reverse transcription PCR (RT-PCR).

28. The tools according to any one of claims 1 through 27, characterized in that the genes or gene sequences are equipped with a labeling or reporter function so that these can be used for other detection methods.

29. Use of the tools according to any one of claims 1 through 27, characterized in that said tools are used for measuring monocyte/macrophage activation or the inflammatory activity in the blood or in cell tissue.
